(19) United States
(12) Patent Application Publication
(10) Pub. No.: US 2011/0263664 A1
(43) Pub. Date: Oct. 27, 2011

(54) Inhibitors of PIM-1 Protein Kinases, Compositions and Methods for Treating Prostate Cancer

(75) Inventors: Charles D. Smith, Mount Pleasant, SC (US); Andrews S. Kraft, Mount Pleasant, SC (US)

(73) Assignee: MUSC FOUNDATION FOR RESEARCH DEVELOPMENT, Charleston, SC (US)

(21) Appl. No.: 12/742,886

(22) PCT Filed: Nov. 14, 2008
(86) PCT No.: PCT/US08/12829

§ 371 Date: Jun. 2, 2011

(30) Foreign Application Priority Data
Nov. 15, 2007 (US) 60988313

(51) Int. Cl. A61K 31/426; C12N 5/00; A61K 31/4166; A61P 35/00; C07D 277/34; C07D 233/96

(52) U.S. Cl. 514/369; 548/183; 548/320.5; 548/319.1; 548/321.1; 548/320.1; 514/391; 435/375

(57) ABSTRACT

The disclosure relates to inhibitors of Pim-1 and/or Pim-2 protein kinase, to compositions comprising one or more inhibitors of Pim-1 and/or Pim-2 protein kinase, and to methods for treating cancer.

### CROSS-REFERENCE TO RELATED APPLICATIONS

This application claims benefit of priority to U.S. Provisional Application Ser. No. 60/988,313 filed Nov. 15, 2007, which is herein incorporated by reference in its entirety.

### FIELD

The disclosure relates to inhibitors of Pim-1 and/or Pim-2 protein kinase, to compositions comprising one or more inhibitors of Pim-1 and/or Pim-2 protein kinase, and to methods for treating cancer. The present disclosure also relates to assays that can be used to screen for compounds that are effective inhibitors of Pim-1 and/or Pim-2 protein kinase.

### BACKGROUND

Pim-1 and Pim-2 are serine/threonine protein kinases that were originally cloned as Proviral Insertions in Murine T cell lymphomas (Selten, G. et al., “Proviaral activation of the putative oncogene Pim-1 in MuLV induced T-cell lyphomas.” Embo J. 4, (7), 1793-8 (1985)). Pim-1 phosphorylates a K/R-K/R-R-K/R-L-S/T sequence (Palaty C. K. et al. (1997) “Phosphorylation site substrate specificity determinants for the Pim-1 protooncogene-encoded protein kinase.” Biochem Cell Biol, 75, 153-62), which shows great similarity to the substrate specificity of the Akt protein kinase family. The Pim-2 gene is 53% identical to Pim-1, with the greatest divergence occurring at the amino and carboxy termini of the encoded proteins. These kinases share the ability to transform lymphoma cells. Pim protein kinases are expressed widely during embryogenesis (Eichmann A. et al. (2000) “Developmental expression of pim kinases suggests functions also outside of the hematopoietic system.” Oncogene, 19, 1215-24) and may play a role in other malignancies (Chiang W. F. et al. (2006) “Up-regulation of a serine-threonine kinase proto-oncogene Pim-1 in oral squamous cell carcinoma.” Int J Oral Maxillofac Surg, 35, 740-5). In transgenic mice, Pim-1 has been show to induce T-cell lymphomas (van Lohuizen M. et al. (1989) “Predisposition to lymphomagenesis in pim-1 transgenic mice: cooperation with c-myc and N-myc in murine leukemia virus-induced tumors.” Cell, 56, 673-82). The Pim protein kinases have been implicated in the development of prostate cancer; DNA microarray analysis demonstrated that Pim-1 is overexpressed in human prostate cancer and its presence correlates with clinical outcomes (Dhanasekaran S. M. et al. (2001) “Delineation of prognostic biomarkers in prostate cancer.” Nature, 412, 822-6). In a mouse model in which elevated levels of c-Myc protein were used to induce the disease, the levels of Pim protein were increased and correlated with the levels of c-Myc (Ellwood-Yen K. et al., (2003) “Myc-driven murine prostate cancer shares molecular features with human prostate tumors.” Cancer Cell, 4, 223-38). In humans, enhanced levels of nuclear Pim-2 in tumor cells has been shown to be associated with a higher risk of PSA recurrence and with perineural invasion of the prostate gland (Dai H. et al. (2005) “Pim-2 upregulation: biological implications associated with disease progression and perinueral invasion in prostate cancer.” Prostate, 65, 276-86). Overexpression of Pim-1 has been reported to be related to the grade of prostate cancer (Xu Y. et al. (2005) “Overexpression of PIM-1 is a potential biomarker in prostate carcinoma.” J Surg Oncol, 92, 326-30). Moderate to strong cytoplasmic staining of Pim-1 was seen in tumors of 68% of patients with a Gleason score of 7 or higher (Valdman A. et al. (2004) “Pim-1 expression in prostatic intraepithelial neoplasia and human prostate cancer.” Prostate, 60, 367-71). Pim-1 also is overexpressed in HGPIN (prostate intraepithelial neoplasia) and Pim staining may be helpful in differentiating benign glands from intraepithelial neoplasia (Cibull T. L. et al. (2006) “Overexpression of Pim-1 during progression of prostatic adenocarcinoma.” J Clin Pathol, 59, 285-8).

Two mechanisms have been implicated in the Pim protein kinase promotion of transformation to date; namely, inhibition of apoptosis and promotion of cell growth. Evidence that Pim functions by preventing cell death through blocking of apoptosis has been gained through analysis of leukemias. The addition of growth factors, including GM-CSF, IL-3 and IL-7, to hematopoietic cells results in an elevation in the levels of Pim protein kinase (Lilly M. et al. (1992) “Sustained expression of the pim-1 kinase is specifically induced in myeloid cells by cytokines whose receptors are structurally related.” Oncogene, 7, 727-32). Conversely, there is an impaired IL-3 and IL-7 response in bone marrow cells that are Pim deficient (Domen J. et al. (1993) “Pim-1 levels determine the size of early B lymphoid compartments in bone marrow.” J Exp Med, 178, 1665-73 and Domen J. et al. (1993) “Impaired interleukin-3 response in Pim-1-deficient bone marrow-derived mast cells.” Blood, 82, 1445-52). It has now been shown that activation of the Jak/STAT pathway by these hormones regulates Pim levels (Shirogane T. et al. (1999) “Synergistic roles for Pim-1 and c-Myc in STAT3-mediated cell cycle progression and antiapoptosis.” Immunity, 11, 709-19 and Stout B. A. et al. (2004) “IL-5 and granulocyte-macrophage colony-stimulating factor activate STAT3 and STAT5 and promote Pim-1 and cyclin D3 protein expression in human eosinophils.” J Immunol, 173, 6409-17).

Control of protein synthesis by the TOR pathway has been shown to play a central role in the control of the transformed phenotype (Bhaskar P. T. et al. (2007) “The two TORCs and Akt.” Dev Cell, 12, 487-502 and Petroulakis E. et al. (2007) “mTOR signaling: implications for cancer and anticancer therapy.” Br J Cancer, 96 Suppl, R11-5). This pathway has already been targeted for therapeutic purposes, with some success already seen in renal cancer (Cho D. et al. (2007) “The role of mammalian target of rapamycin inhibitors in the treatment of advanced renal cancer.” Clin Cancer Res, 13, 758s-763s). The TOR protein kinase is found in two complexes, TORC1 and TORC2. The TORC1 complex controls protein synthesis by phosphorylating the 4E-BP1 protein at threonine 37 and 46. This phosphorylation releases 4E-BP1 from eIF4E allowing cap-dependent transcription to take place. TORC1 also phosphorylates p70S6 protein kinase, which on activation phosphorylates the S6 protein, and this is critical for translation. In contrast, the TORC2 complex phosphorylates S473 of the Akt protein kinase allowing a second phosphorylation by the PDK1 kinase at T308 to occur and for Akt to be activated.

### SUMMARY

The present disclosure provides a method for treating cancer by administering to a human an effective amount of one or more of the compounds as disclosed herein. The present disclosure further relates to pharmaceutical compositions comprising an effective amount of one or more Pim-1 and/or Pim-2 inhibitors as disclosed herein. The present disclosure further relates to methods of inhibiting Pim-1 and/or Pim-2 in vitro, in vivo, and ex vivo. The present disclosure further relates to novel compounds suitable for use in treating cancer and for use in pharmaceutical compositions that are used to treat cancer.

The disclosed compounds have been found to block the ability of Pim kinases to phosphorylate peptides with IC₅₀s in the nanomolar range, and inhibit the Pim protein kinase directed phosphorylation of two known substrates, 4E-BP1 and p27^{Kip1}. The disclosed compounds can be Pim1, or Pim2 specific, or dual inhibitors blocking the activity of both of these enzymes.

The disclosed compound when exposed to two different prostate cancer cell lines inhibited the ability of Pim kinase to phosphorylate the proapoptotic Bad protein on serine 112. Phosphorylated Bad protein is sequestered by 14-3-3 proteins which blocks its ability to cause apoptotic cell death. As such, Pim promotes survival of chemotherapy treated prostate cancer, regulates cardiomyocyte survival, and T cell survival. The disclosed compounds therefore provide a method for reversing the prosurvival phenotype induced by Pim overexpression, thereby providing compositions that are useful as chemotherapeutic agents in tumors with enhanced survival secondary to overexpression of this enzyme.

In addition, when the disclosed compounds are combined with immunosuppressants, inter alia, rapamycin, the resistance afforded hematopoietic cells by Pim kinases is reduced. As such, the combination of the disclosed Pim inhibitors and mTOR inhibitors provides a treatment option for hematological malignancies and other tumor types that demonstrate reduced sensitivity to rapamycin.

### BRIEF DESCRIPTION OF THE FIGURES

FIG. 1 depicts the dose response curve for inhibition of Pim-1 protein kinase by 5-(3-trifluoromethylbenzylidene)thiazolidine-2,4-dione (D5). His-tagged 4E-BP-1 was incubated with 0.1 μg Pim-1 protein kinase for 1 hour at 30° C. together with [γ-³²P]ATP, Mg²⁺, and cold ATP with from 0.125 to 3 μM of D5.

FIG. 2 indicates 5-(3-trifluoromethylbenzylidene)thiazolidine-2,4-dione (D5) acts as a competitive inhibitor with respect to ATP. Pim-1 kinase assays were performed as described in FIG. 1 with the indicated concentrations of ATP and D5.

FIG. 3 depicts the Lineweaver-Burke plot for the varying concentrations as shown in FIG. 2. Pim-1 kinase activity was measured using the coupled assay in the presence of the indicated concentrations of ATP and 0 (), 5 (▪) or 10 (▴) μM D5.

FIG. 4 indicates that 5-(3-trifluoromethylbenzylidene)thiazolidine-2,4-dione (D5) enhances rapamycin inhibition of 4E-BP-1 phosphorylation and increases rapamycin-induced AKT 473 phosphorylation.

FIG. 5 indicates that the addition of 5-(3-trifluoromethylbenzylidene)thiazolidine-2,4-dione (D5) with or without rapamycin inhibits the growth of PC-3 prostate cancer cells.

FIG. 6 depicts the effect of compounds disclosed herein when administered with PKC412 on MV7;11 cells (human leukemic cell line containing the FLT3/ITD mutation).

FIG. 7 depicts the effect of compound D16 on tumor growth. Female Balb/C mice were injected subcutaneously with JC cells (1×10⁶) suspended in PBS. After palpable tumor growth, animals were treated five days per week by intraperitoneal injection of vehicle alone (◯) or 50 mg/kg of D16 (). Values represent the mean±standard error tumor volumes. n=5 mice per group.

FIG. 8 depicts the growth inhibition of the cell lines PC3, DU145, LNCaP, U937, K582, and MV7;11 by D5 and D16.

FIG. 9 depicts that DU145 cells are more sensitive to D5 and D16 under serum-free conditions.

FIG. 10 depicts that the 22Rv1-vector cells show more endogenous Pim-1 protein compared to DU145-vector cells when treated with D5 and D16.

FIG. 11 depicts that more endogenous phosphorylated Bad protein (phosphoBad) is present when treated with D5 and D16.

FIG. 12 depicts that there is a significant reduction in phosphoBad levels in D5-treated FDCP1-Pim cells by 2 hours compared to DMSO-treated cells.

FIG. 13 depicts that D5 and D16 caused a significant G1 cell cycle arrest in cell lines DU145 and MV7;11 as compared to a DMSO control.

FIG. 14 depicts the results for cells treated with DMSO or D5 (5 μM) for 72 hours under serum-free conditions.

FIG. 15 depicts the ability of Pim-1 to phosphorylate p27^{Kip1} and the ability of D5 and D16 (5 μM) to reduce phosphorylation of this substrate in vitro.

FIG. 16 depicts the increase in the amount of p27^{Kip1} in the leukemic cell lines K562, U937, and MV7;11 after treatment with D5 or D16 for 72 hours in media containing 10% FCS, followed by detection of p27^{Kip1} levels in cytoplasmic and nuclear fractions.

FIG. 17 depicts that when K562 cells were treated under the same conditions as FIG. 16, Cdk2 was immunoprecipitated from D5 or D16 treated cells and showed approximately 50% and 60% respectively decreased activity.

FIG. 18 depicts DU145-vector and DU145-Pim cells transfected with a plasmid expressing p27^{Kip1} fused to enhanced yellow fluorescent protein (EYFP) and p27^{Kip1} when treated with D5 and D16 indicate that the control vector expressing EYFP alone is distributed throughout the nucleus and cytosol while the fusion with p27^{Kip1} localizes the fluorescence in the nucleus as demonstrated by overlay with Hoescht dye which stains nuclei.

FIG. 19 depicts the Western blot obtain from K562 leukemia cells transfected with HA-tagged p27^{Kip1}.

FIG. 20 depicts that the mutation of either T157 or T198 to alanine resulted in a mutant p27^{Kip1} that localized exclusively to the nucleus in K562 cells demonstrating similar results to the Pim-1 overexpressing DU145 cells.

FIG. 21 depicts that compounds D5 and 5-(4-propoxybenzylidene)thiazolidine-2,4-dione (D16) act synergistically with rapamycin to inhibit cell growth. D5 and D16 combined with rapamycin effectively reduce the level of phospho4EBP1 (T37/46). FDCP-1 cells were starved of IL-3 and serum for 1 h during which cells were treated with rapamycin or D5 or a combination of the two agents. After 1 h of treatment IL-3 (2 ng/mL) was added for 5 min to stimulate 4E-BP1 phosphorylation. Cells were pelleted and the level of phospho4EBP1 (T37/46), 4E-BP1 and GAPDH determined by SDS-PAGE followed by immunoblotting.

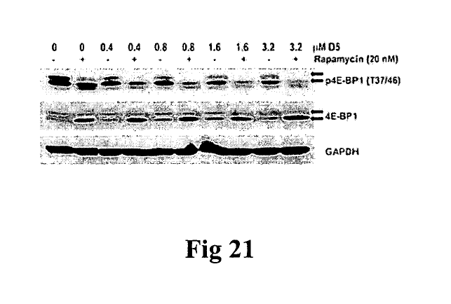

FIG. 22 depicts that the combination of D5 or D16 with rapamycin effectively inhibits the growth of MV7;11 (left) and FDCP1 (right) cells. Cells were incubated for 72 h in RPMI+10% FCS (IL-3 included in FDCP1 cells) with rapamycin (5 nM), D5 (5 μM), D16 (5 μM) or the combination. Data are represented as the percent growth inhibition relative to DMSO and are the average of 4 independent experiments with the standard deviation from the mean (SEM?) shown.

FIG. 23 depicts the combination index values demonstrate synergism between rapamycin and D5 or D16 in MV7;11 cells.

### DETAILED DESCRIPTION

In this specification and in the claims that follow, reference will be made to a number of terms, which shall be defined to have the following meanings:

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

An organic radical can have, for example, 1-26 carbon atoms, 1-18 carbon atoms, 1-12 carbon atoms, 1-8 carbon atoms, or 1-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Substituted and unsubstituted linear, branched, or cyclic alkyl units include the following non-limiting examples: methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), cyclobutyl (C₄), cyclopentyl (C₅), cyclohexyl (C₆), and the like; whereas substituted linear, branched, or cyclic alkyl, non-limiting examples of which includes, hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 2,2,2-trifluoroethyl (C₃), 3-carboxypropyl (C₃), 2,3-dihydroxycyclobutyl (C₄), and the like.

Substituted and unsubstituted linear, branched, or cyclic alkenyl include, ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.

Substituted and unsubstituted linear or branched alkynyl include, ethynyl (C₂), prop-2-ynyl (also propargyl) (C₃), propyn-1-yl (C₃), and 2-methyl-hex-4-yn-1-yl (C₇); substituted linear or branched alkynyl, non-limiting examples of which include, 5-hydroxy-5-methylhex-3-ynyl (C₇), 6-hydroxy-6-methylhept-3-yn-2-yl (C₈), 5-hydroxy-5-ethylhept-3-ynyl (C₉), and the like.

The term “aryl” as used herein denotes organic rings that consist only of a conjugated planar carbon ring system with delocalized pi electrons, non-limiting examples of which include phenyl (C₆), naphthylen-1-yl (C₁₀), naphthylen-2-yl (C₁₀). Aryl rings can have one or more hydrogen atoms substituted by another organic or inorganic radical. Non-limiting examples of substituted aryl rings include: 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyanonaphthylen-1-yl (C₁₀).

The term “heteroaryl” denotes an aromatic ring system having from 5 to 10 atoms. The rings can be a single ring, for example, a ring having 5 or 6 atoms wherein at least one ring atom is a heteroatom not limited to nitrogen, oxygen, or sulfur. Or “heteroaryl” can denote a fused ring system having 8 to 10 atoms wherein at least one of the rings is an aromatic ring and at least one atom of the aromatic ring is a heteroatom not limited nitrogen, oxygen, or sulfur.

The following are non-limiting examples of heteroaryl rings according to the present disclosure:

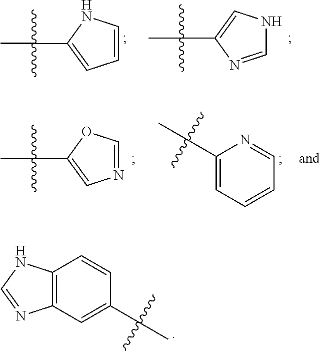

The term “heterocyclic” denotes a ring system having from 3 to 10 atoms wherein at least one of the ring atoms is a heteroatom not limited to nitrogen, oxygen, or sulfur. The rings can be single rings, fused rings, or bicyclic rings. Non-limiting examples of heterocyclic rings include:

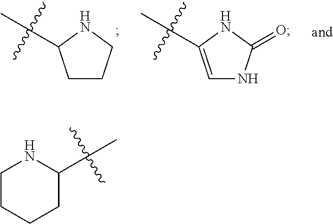

All of the aforementioned heteroaryl or heterocyclic rings can be optionally substituted with one or more substitutes for hydrogen as described herein further. it unambiguous to the artisan of ordinary skill which rings are referred to herein.

The term “substituted” is used throughout the specification. The term “substituted” is defined herein as a unit, whether acyclic or cyclic, that has one or more hydrogen atoms replaced by one or more units as defined further herein.

For the purposes of the present disclosure the terms “compound,” “analog,” and “composition of matter” stand equally well for the chemical entities described herein, including all enantiomeric forms, diastereomeric forms, salts, and the like, and the terms “compound,” “analog,” and “composition of matter” are used interchangeably throughout the present specification.

### Compositions of Matter

One embodiment relates to novel compositions of matter that are Pim-1 and/or Pim-2 inhibitors. These inhibitors disclosed herein have the formulae:

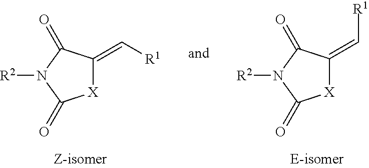

wherein X is S or NR³;
R³ is benzyl or benzyl substituted by from 1 to 5 independently chosen organic radicals;
R¹ is phenyl or phenyl substituted by from 1 to 5 independently chosen organic radicals; and
R² is chosen from:
i) hydrogen;
ii) C₁-C₄ linear, branched, or cyclic alkyl; and
iii) benzyl or benzyl substituted by from 1 to 5 independently chosen organic radicals with the proviso the compound is not:
5-(3-trifluoromethylbenzylidene)thiazolidine-2,4-dione;
5-(3-trifluoromethoxybenzylidene)thiazolidine-2,4-dione; or
5-(4-trifluoromethylbenzylidene)thiazolidine-2,4-dione.

The compounds of the present disclosure can be present as individual isomers, for example, the (Z) or (E) isomer or as a mixture of the (Z) and (E) isomers.

The compounds disclosed herein also include all salt forms, for example, salts of both basic groups, inter alia, amines, as well as salts of acidic groups, inter alia, carboxylic acids. The following are non-limiting examples of anions that can form salts with basic groups, for example, chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, citrate, and the like. The following are non-limiting examples of cations that can form salts of acidic groups, for example, sodium, lithium, potassium, calcium, magnesium, bismuth, and the like. The counter ions are present in a sufficient amount to provide electronic neutrality.

A first embodiment relates to compounds wherein the at least one organic radical is chosen from —CH₂F, —CHF₂, —CF₃, —CH₂CH₂F, —CH₂CHF₂, —CH₂CF₃, —CHFCH₃, —CF₂CH₃, —CHFCH₂F, —CF₂CH₂F, —CF₂CHF₂, and —CF₂CF₃.

Another embodiment relates to compounds wherein at least one organic radical is chosen from —OCH₂F, —OCHF₂, —OCF₃, —OCH₂CH₂F, —OCH₂CHF₂, —OCH₂CF₃, —OCHFCH₃, —OCF₂CH₃, —OCHFCH₂F, —OCF₂CH₂F, —OCF₂CHF₂, and —OCF₂CF₃.

A further embodiment relates to compounds wherein at least one organic radical is chosen from —CH₂Cl, —CHCl₂, —CCl₃, —CH₂CH₂Cl, —CH₂CHCl₂, —CH₂CCl₃, —CHClCH₃, —CCl₂CH₃, —CHClCH₂Cl, —CCl₂CH₂Cl, —CCl₂CHCl₂, and —CCl₂CCl₃.

A yet further embodiment relates to compounds wherein at least one organic radical is chosen from —OCH₂Cl, —OCHCl₂, —OCCl₃, —OCH₂CH₂Cl, —OCH₂CHCl₂, —OCH₂CCl₃, —OCHClCH₃, —OCCl₂CH₃, —OCHClCH₂Cl, —OCCl₂CH₂Cl, —OCCl₂CHCl₂, and —OCCl₂CCl₃.

A yet further embodiment relates to compounds having the formula:

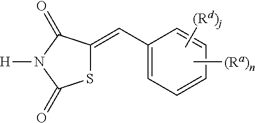

wherein R^{a} is an organic radical;
R^{d} is an organic radical chosen from haloalkyl and haloalkoxy;
the index n is from 0 to 4; and the index j is from 1 to 5.

A still further embodiment relates to compounds having the formula:

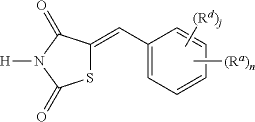

wherein the index n is from 0 to 4;
the index j is from 1 to 5;
R^{a} is from 0 to 4 organic radicals that are substitutions for hydrogen independently chosen from:
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{4a})(R^{4b})]_{y}OR⁵;
   wherein R⁵ is chosen from:
   a) —H;
   b) C₁-C₁₂ substituted or unsubstituted linear, branched, or cyclic alkyl or C₁-C₁₂ substituted or unsubstituted linear, branched, or cyclic haloalkyl;
   c) C₆ or C₁₀ substituted or unsubstituted aryl or C₇-C₂₀ alkylenearyl;
   d) C₁-C₉ substituted or unsubstituted heterocyclic; and
   e) C₁-C₁₁ substituted or unsubstituted heteroaryl;
viii) —[C(R^{4a})(R^{4b})]_{y}N(R^{6a})(R^{6b});
   wherein R^{6a} and R^{6b} are each independently chosen from:
   a) —H;
   b) —OR⁷;
      R⁷ is hydrogen or C₁-C₄ linear alkyl;
   c) C₁-C₁₂ substituted or unsubstituted linear, branched, or cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; and
   g) R^{6a} and R^{6b} can be taken together to form a substituted or unsubstituted ring having from 3 to 10 carbon atoms and from 0 to 3 heteroatoms chosen from oxygen, nitrogen, and sulfur;
ix) —[C(R^{4a})(R^{4b})]_{y}C(O)R⁸;
   wherein R⁸ is chosen from:
   a) C₁-C₁₂ substituted or unsubstituted linear, branched, or cyclic alkyl;
   b) —OR⁹;
      wherein R⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
   c) —N(R^{10a})(R^{10b});
      wherein R^{10a} and R^{10b} are each independently hydrogen, C₁-C₁₂ substituted or unsubstituted linear, branched, or cyclic alkyl; C₆ or C-₁₀ substituted or unsubstituted aryl; C₁-C₉ substituted or unsubstituted heterocyclic; C₁-C₁₁ substituted or unsubstituted heteroaryl; or R^{10a} and R^{10b} can be taken together to form a substituted or unsubstituted ring having from 3 to 10 carbon atoms and from 0 to 3 heteroatoms chosen from oxygen, nitrogen, and sulfur;
x) —[C(R^{4a})(R^{4b})]_{y}OC(O)R¹¹;
   wherein R¹¹ is chosen from:
   a) C₁-C₁₂ substituted or unsubstituted linear, branched, or cyclic alkyl; and
   b) —N(R^{12a})(R^{12b});
      R^{12a} and R^{12b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{12a} and R^{12b} can be taken together to form a substituted or unsubstituted ring having from 3 to 10 carbon atoms and from 0 to 3 heteroatoms chosen from oxygen, nitrogen, and sulfur;
xi) —[C(R^{4a})(R^{4b})]_{y}NR¹³C(O)R¹⁴;
   wherein R¹³ is chosen from:
   a) —H; and
   b) C₁-C₄ substituted or unsubstituted linear, branched, or cyclic alkyl;
   wherein R¹⁴ is chosen from:
   a) C₁-C₁₂ substituted or unsubstituted linear, branched, or cyclic alkyl; and
   b) —N(R^{15a})(R^{15b});
      R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
xii) —[C(R^{4a})(R^{4b})]_{y}CN;
xiii) —[C(R^{4a})(R^{4b})]_{y}NO₂;
xiv) —[C(R^{4a})(R^{4b})]_{y}SO₂R¹⁶;
   R¹⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl; and
xv) halogen;
   R^{4a} and R^{4b} are each independently hydrogen or C₁-C₄ alkyl; and
   the index y is from 0 to 5;
   each R^{d} is independently an organic radical having the formula:

—[C(H)ₐ(Z)_{b}]_{d}C(H)ₑ(Z)_{f} or —O[C(H)ₐ(Z)_{b}]_{d}C(H)ₑ(Z)_{f}

Z is halogen; and
the index a is from 0 to 2; the index b is from 0 to 2; the index d is from 0 to 6; the index e is from 0 to 3; the index f is from 0 to 3; with the proviso that the indices b and f are not both equal to 0.

The following are non-limiting examples of the disclosed compounds that are Pim-1 and/or Pim-2 inhibitors according to the present disclosure:
5-[3-(1-fluoroethoxy)benzylidene]thiazolidine-2,4-dione;
5-[3-(1,1-difluoroethoxy)benzylidene]thiazolidine-2,4-dione;
5-[3-(1,1,2-trifluoroethoxy)benzylidene]thiazolidine-2,4-dione;
5-[3-(1,1,2,2-tetrafluoroethoxy)benzylidene]thiazolidine-2,4-dione
5-[3-(1,1,2,2,2-pentafluoroethoxy)benzylidene]thiazolidine-2,4-dione;
5-[3-(2-fluoroethoxy)benzylidene]thiazolidine-2,4-dione;
5-[3-(2,2-difluoroethoxy)benzylidene]thiazolidine-2,4-dione;
5-[3-(2,2,2-trifluoroethoxy)benzylidene]thiazolidine-2,4-dione;
5-[3-(1,2,2,2-tetrafluoroethoxy)benzylidene]thiazolidine-2,4-dione;
5-[4-(1-fluoroethoxy)benzylidene]thiazolidine-2,4-dione;
5-[4-(1,1-difluoroethoxy)benzylidene]thiazolidine-2,4-dione;
5-[4-(1,1,2-trifluoroethoxy)benzylidene]thiazolidine-2,4-dione;
5-[4-(1,1,2,2-tetrafluoroethoxy)benzylidene]thiazolidine-2,4-dione
5-[4-(1,1,2,2,2-pentafluoroethoxy)benzylidene]thiazolidine-2,4-dione;
5-[4-(2-fluoroethoxy)benzylidene]thiazolidine-2,4-dione;
5-[4-(2,2-difluoroethoxy)benzylidene]thiazolidine-2,4-dione;
5-[4-(2,2,2-trifluoroethoxy)benzylidene]thiazolidine-2,4-dione;
5-[4-(1,2,2,2-tetrafluoroethoxy)benzylidene]thiazolidine-2,4-dione;
5-(2-trifluoromethoxybenzylidene)thiazolidine-2,4-dione
5-(4-trifluoromethoxybenzylidene)thiazolidine-2,4-dione;
5-(2-trifluoromethylbenzylidene)thiazolidine-2,4-dione;
5-[2,3-di(trifluoromethyl)benzylidene]thiazolidine-2,4-dione;
5-[2,4-di(trifluoromethyl)benzylidene]thiazolidine-2,4-dione;
5-[2,5-di(trifluoromethyl)benzylidene]thiazolidine-2,4-dione;
5-[2,6-di(trifluoromethyl)benzylidene]thiazolidine-2,4-dione;
5-[2,3-di(trifluoromethoxy)benzylidene]thiazolidine-2,4-dione;
5-[2,4-di(trifluoromethoxy)benzylidene]thiazolidine-2,4-dione;
5-[2,5-di(trifluoromethoxy)benzylidene]thiazolidine-2,4-dione;
5-[2,6-di(trifluoromethoxy)benzylidene]thiazolidine-2,4-dione;
5-[3-fluoro-2-(trifluoromethyl)benzylidene]thiazolidine-2,4-dione;
5-[4-fluoro-2-(trifluoromethyl)benzylidene]thiazolidine-2,4-dione;
5-[5-fluoro-2-(trifluoromethyl)benzylidene]thiazolidine-2,4-dione;
5-[6-fluoro-2-(trifluoromethyl)benzylidene]thiazolidine-2,4-dione;
5-[2-fluoro-3-(trifluoromethyl)benzylidene]thiazolidine-2,4-dione;
5-[4-fluoro-3-(trifluoromethyl)benzylidene]thiazolidine-2,4-dione;
5-[5-fluoro-3-(trifluoromethyl)benzylidene]thiazolidine-2,4-dione;
5-[6-fluoro-3-(trifluoromethyl)benzylidene]thiazolidine-2,4-dione;
5-[2-fluoro-4-(trifluoromethyl)benzylidene]thiazolidine-2,4-dione;
5-[3-fluoro-4-(trifluoromethyl)benzylidene]thiazolidine-2,4-dione;
5-[5-fluoro-4-(trifluoromethyl)benzylidene]thiazolidine-2,4-dione;
5-[6-fluoro-4-(trifluoromethyl)benzylidene]thiazolidine-2,4-dione;
5-[3-fluoro-2-(trifluoromethoxy)benzylidene]thiazolidine-2,4-dione;
5-[4-fluoro-2-(trifluoromethoxy)benzylidene]thiazolidine-2,4-dione;
5-[5-fluoro-2-(trifluoromethoxy)benzylidene]thiazolidine-2,4-dione;
5-[6-fluoro-2-(trifluoromethoxy)benzylidene]thiazolidine-2,4-dione;
5-[2-fluoro-3-(trifluoromethoxy)benzylidene]thiazolidine-2,4-dione;
5-[4-fluoro-3-(trifluoromethoxy)benzylidene]thiazolidine-2,4-dione;
5-[5-fluoro-3-(trifluoromethoxy)benzylidene]thiazolidine-2,4-dione;
5-[6-fluoro-3-(trifluoromethoxy)benzylidene]thiazolidine-2,4-dione;
5-[2-fluoro-4-(trifluoromethoxy)benzylidene]thiazolidine-2,4-dione;
5-[3-fluoro-4-(trifluoromethoxy)benzylidene]thiazolidine-2,4-dione;
5-[5-fluoro-4-(trifluoromethoxy)benzylidene]thiazolidine-2,4-dione; and
5-[6-fluoro-4-(trifluoromethoxy)benzylidene]thiazolidine-2,4-dione.

A further embodiment of the disclosure relates to compounds having the formula:

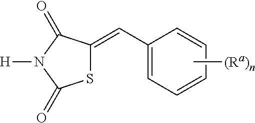

wherein the index n is from 0 to 5 and R^{a} represents from 1 to 5 optionally present and independently chosen organic radicals that are substitutions for hydrogen.

Another embodiment of the disclosure relates to compounds having the formula:

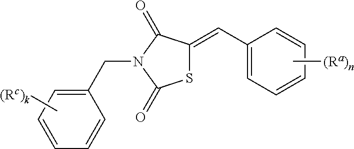

wherein the indices k and n are each independently from 0 to 5 and R^{a} and R^{c} each represents from 1 to 5 optionally present and independently chosen organic radicals that are substitutions for hydrogen.

A further embodiment of the disclosure relates to compounds having the formula:

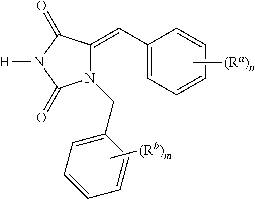

wherein the indices m and n are each independently from 0 to 5 and R^{a} and R^{b} each independently represent from 1 to 5 optionally present and independently chosen organic radicals that are substitutions for hydrogen.

A yet further embodiment of the disclosure relates to compounds having the formula:

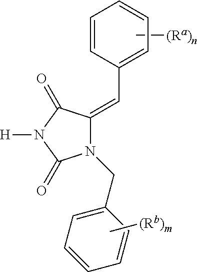

wherein the indices m and n are each independently from 0 to 5 and R^{a} and R^{b} each independently represent from 1 to 5 optionally present and independently chosen organic radicals that are substitutions for hydrogen.

A still further embodiment of the disclosure relates to compounds having the formula:

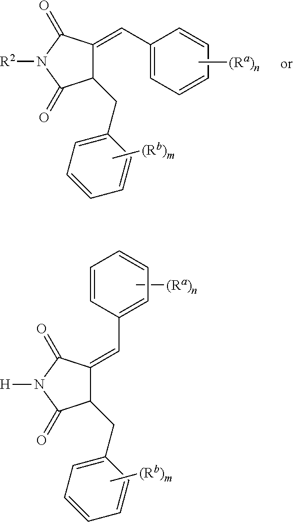

wherein the indices m and n are each independently from 0 to 5 and R^{a} and R^{b} each independently represent from 1 to 5 optionally present and independently chosen organic radicals that are substitutions for hydrogen and R² is C₁-C₄ linear, branched, or cyclic alkyl.

### R¹ Units

The following describes R¹ units that comprise the compounds suitable for use in treating cancer. R¹ units are phenyl or phenyl substituted by from 1 to 5 independently chosen R^{a} units wherein R^{a} units are organic radicals. Non-limiting examples of organic radicals are chosen from:
i) C₁-C₁₂ substituted or unsubstituted linear, branched, or cyclic alkyl; for example, methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), cyclobutyl (C₄), cyclopentyl (C₅), cyclohexyl (C₆);
ii) C₂-C₁₂ substituted or unsubstituted linear, branched, or cyclic alkenyl; for example, ethenyl (C₂), 3-propenyl (C₃), 1-propenyl (also 2-methylethenyl) (C₃), isopropenyl (also 2-methylethen-2-yl) (C₃), buten-4-yl (C₄);
iii) C₂-C₁₂ substituted or unsubstituted linear or branched alkynyl; for example, ethynyl (C₂), prop-2-ynyl (also propargyl) (C₃), propyn-1-yl (C₃);
iv) C₁-C₁₂ substituted or unsubstituted linear or branched haloalkyl; for example, —CH₂F, —CHF₂, —CF₃, —CH₂CH₂F, —CH₂CF₃, and —CF₂CF₃;
iv) C₆ or C₁₀ substituted or unsubstituted aryl; for example, phenyl, naphthyl (also referred to herein as naphthylen-1-yl (C₁₀) or naphthylen-2-yl (C₁₀));
v) C₁-C₉ substituted or unsubstituted heterocyclic; as described herein below;
vi) C₁-C₁₁ substituted or unsubstituted heteroaryl; as described herein below;
vii) —[C(R^{4a})(R^{4b})]_{y}OR⁵;
   wherein R⁵ is chosen from:
   a) —H;
   b) C₁-C₁₂ substituted or unsubstituted linear, branched, or cyclic alkyl or C₁-C₁₂ substituted or unsubstituted linear, branched, or cyclic haloalkyl;
   c) C₆ or C₁₀ substituted or unsubstituted aryl or C₇-C₂₀ alkylenearyl;
   d) C₁-C₉ substituted or unsubstituted heterocyclic; and
   e) C₁-C₁₁ substituted or unsubstituted heteroaryl;
   for example, —OH, —CH₂OH, —OCH₃, —OCF₃, —CH₂OCH₃, —OCH₂CH₃, —CH₂OCH₂CH₃, —OCH₂CH₂CH₃, —CH₂OCH₂CH₂CH₃, —CH₂OC₆H₅, and —CH₂OC₆H₅;
viii) —[C(R^{4a})(R^{4b})]_{y}N(R^{6a})(R^{6b});
   wherein R^{6a} and R^{6b} are each independently chosen from:
   a) —H;
   b) —OR⁷;
      R⁷ is hydrogen or C₁-C₄ linear alkyl;
   c) C₁-C₁₂ substituted or unsubstituted linear, branched, or cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; and
   g) R^{6a} and R^{6b} can be taken together to form a substituted or unsubstituted ring having from 3 to 10 carbon atoms and from 0 to 3 heteroatoms chosen from oxygen, nitrogen, and sulfur; for example, —NH₂, —CH₂NH₂, —NHCH₃, —N(CH₃)₂, —NH(CH₂CH₃), —CH₂NHCH₃, —CH₂N(CH₃)₂, —CH₂NH(CH₂CH₃), —NHOH, and —CH₂NHOH;
ix) —[C(R^{4a})(R^{4b})]_{y}C(O)R⁸;
   wherein R⁸ is chosen from:
   a) C₁-C₁₂ substituted or unsubstituted linear, branched, or cyclic alkyl;
   b) —OR⁹;
      R⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
   c) —N(R^{10a})(R^{10b});
      R^{10a} and R^{10b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{10a} and R^{10b} can be taken together to form a substituted or unsubstituted ring having from 3 to 10 carbon atoms and from 0 to 3 heteroatoms chosen from oxygen, nitrogen, and sulfur;
      for example, —COCH₃, —CO₂H, —CO₂CH₃, —CONH₂, —CH₂COCH₃, —CH₂CO₂H, —CH₂CO₂CH₃, —CH₂CONH₂, —CONHCH₃, —CO₂CH₂CH₃, —CH₂CONHCH₃, and —CON(CH₃)₂;
x) —[C(R^{4a})(R^{4b})]_{y}OC(O)R¹¹;
   wherein R¹¹ is chosen from:
   a) C₁-C₁₂ substituted or unsubstituted linear, branched, or cyclic alkyl; and
   b) —N(R^{12a})(R^{12b});
      R^{12a} and R^{12b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{117a} and R^{12b} can be taken together to form a substituted or unsubstituted ring having from 3 to 10 carbon atoms and from 0 to 3 heteroatoms chosen from oxygen, nitrogen, and sulfur;
      for example, —OC(O)CH₃, —OC(O)CH₂CH₃, —CH₂OC(O)CH₃, —OC(O)NH₂, —CH₂OC(O)NH₂, and —CH₂OC(O)N(CH₃)₂;
xi) —[C(R^{4a})(R^{4b})]_{y}NR¹³C(O)R¹⁴
   wherein R¹³ is chosen from:
   a) —H; and
   b) C₁-C₄ substituted or unsubstituted linear, branched, or cyclic alkyl;
   wherein R¹⁴ is chosen from:
   a) C₁-C₁₂ substituted or unsubstituted linear, branched, or cyclic alkyl; and
   b) —N(R^{15a})(R^{15b});
      R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
      for example, —NHC(O)CH₃, —NHC(O)CH₂CH₃, —CH₂NHC(O)CH₃, —NHC(O)NH₂, and —CH₂NHC(O)N(CH₃)₂;
xii) —[C(R^{4a})(R^{4b})]_{y}CN;
xiii) —[C(R^{4a})(R^{4b})]_{y}NO₂;
xiv) —[C(R^{4a})(R^{4b})]_{y}SO₂R¹⁶;
   R¹⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
   for example, —SO₂H, —CH₂SO₂H, —SO₂CH₃, —CH₂SO₂CH₃, —SO₂C₆H₅, and —CH₂SO₂C₆H₅; and
xv) halogen; —F, —Cl, —Br, and —I;
   R^{4a} and R^{4b} are each independently hydrogen or C₁-C₄ alkyl.

The index y can have any value from 0 to 6, for example, y can be 0, 1, 2, 3, 4, 5, or 6.

A first embodiment of the R¹ units of the disclosure relates to compounds wherein R¹ is phenyl.

Another embodiment of the R¹ units of the disclosure relates to compounds wherein R¹ is substituted by from 1 to 5 organic radicals independently chosen from:
i) C₁-C₄ linear, branched, or cyclic alkyl;
ii) C₁-C₄ haloalkyl;
iii) phenyl;
iv) —OR⁵;
   wherein R⁵ is chosen from:
   a) —H; and
   b) C₁-C₄ linear or branched alkyl or C₁-C₄ linear or branched haloalkyl;
v) —N(R^{6a})(R^{6b});
   wherein R^{6a} and R^{6b} are each independently chosen from:
   a) —H; and
   b) C₁-C₄ linear or branched alkyl;
vi) —C(O)R⁸;
   wherein R⁸ is chosen from:
   a) C₁-C₄ linear or branched alkyl;
   b) —OR⁹;
      R⁹ is hydrogen or C₁-C₄ linear alkyl; and
   c) —N(R^{10a})(R^{10b});
   R^{10a} and R^{10b} are each independently hydrogen or C₁-C₄ linear alkyl;
vii) —OC(O)R¹¹; R¹¹ is C₁-C₄ linear or branched alkyl or phenyl;
viii) —CN;
ix) —NO₂;
x) —SO₂R¹⁶; R¹⁶ is hydrogen, hydroxyl, or C₁-C₄ linear or branched alkyl; and
xi) halogen.

One iteration of this embodiment relates to compounds having the formula:

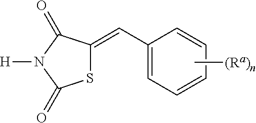

wherein the index n is from 1 to 5.

One example of this iteration relates to compounds wherein R^{a} is C₁-C₄ linear, branched, or cyclic alkyl, for example, the compounds having the formulae:

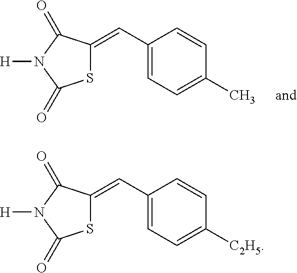

Another example of this iteration relates to compounds wherein r is C₁-C₄ linear, branched, or cyclic haloalkyl, for example, the compounds having the formulae:

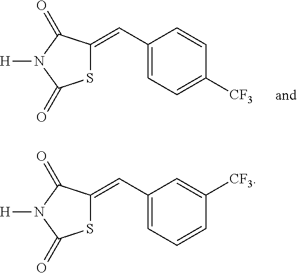

A further example of this iteration relates to compounds wherein R^{a} is C₁-C₄ linear, branched, or cyclic alkoxy, for example, the compounds having the formulae:

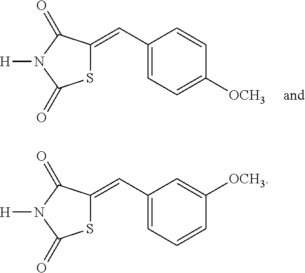

A still further example of this iteration relates to compounds wherein R^{a} is C₁-C₄ linear, branched, or cyclic haloalkoxy, for example, the compounds having the formulae:

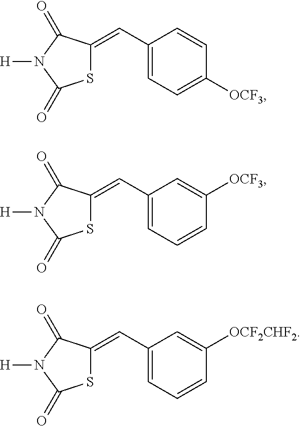

A yet further example of this iteration relates to compounds wherein R^{a} is halogen, for example, the compounds having the formulae:

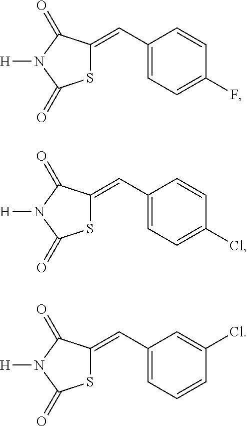

A still yet further example of this iteration relates to compounds wherein R^{a} is amino or alkyl amino, for example, the compounds having the formulae:

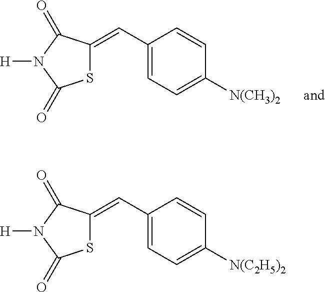

A further embodiment of the disclosed compounds relates to compounds having the formula:

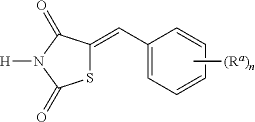

wherein each R^{a} represents from 1 to 5 optionally present organic radicals independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

A yet further embodiment of the disclosed compounds relates to compounds having the formulae:

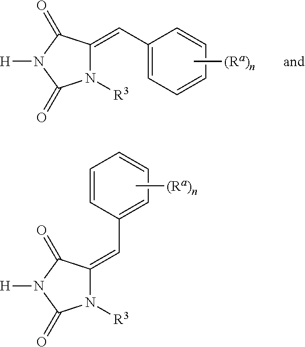

wherein each R^{a} represents from 1 to 5 organic radicals independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃;
   and R³ is further defined herein below.

One example of this iteration relates to compounds wherein R^{a} is C₁-C₄ linear, branched, or cyclic alkyl, for example, the compounds having the formulae:

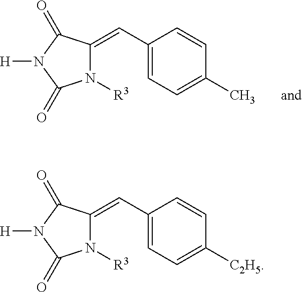

Another example of this iteration relates to compounds wherein R^{a} is C₁-C₄ linear, branched, or cyclic haloalkyl, for example, the compounds having the formulae:

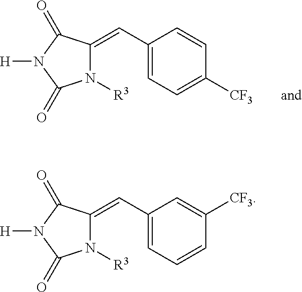

A further example of this iteration relates to compounds wherein R^{a} is C₁-C₄ linear, branched, or cyclic alkoxy, for example, the compounds having the formulae:

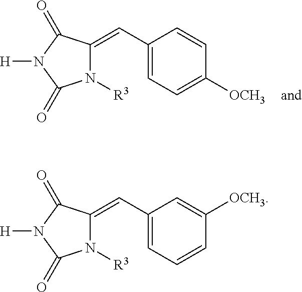

A still further example of this iteration relates to compounds wherein R^{a} is C₁-C₄ linear, branched, or cyclic haloalkoxy, for example, the compounds having the formulae:

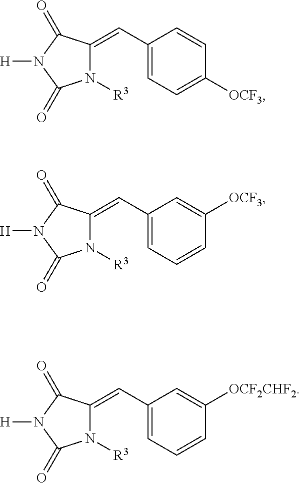

A yet further example of this iteration relates to compounds wherein R^{a} is halogen, for example, the compounds having the formulae:

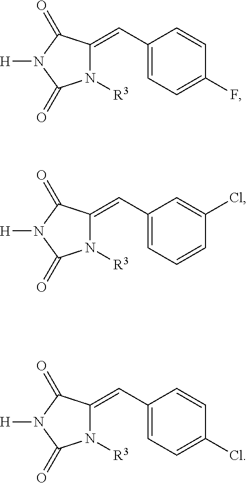

A still yet further example of this iteration relates to compounds wherein R^{a} is amino or alkyl amino, for example, the compounds having the formulae:

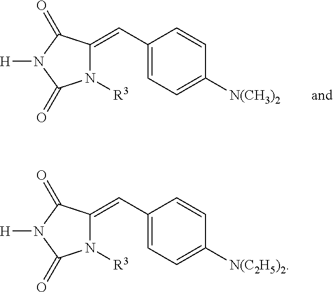

### R² Units

The following describes R² units that comprise the compounds suitable for use in treating cancer.

R² units are chosen from:
i) hydrogen;
ii) methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), and cyclobutyl (C₄); and
iii) benzyl or benzyl substituted by from 1 to 5 organic radicals.

Non-limiting examples of organic radicals that can substitute for hydrogen atoms of R² benzyl units include:
i) C₁-C₄ linear, branched, or cyclic alkyl;
ii) C₁-C₄ haloalkyl;
iii) phenyl;
iv) —OR¹⁷;
   wherein R¹⁷ is chosen from:
   a) —H; and
   b) C₁-C₄ linear or branched alkyl or C₁-C₄ linear or branched haloalkyl;
v) —N(R^{18a})(R^{18b});
   wherein R^{18a} and R^{18b} are each independently chosen from:
   a) —H; and
   b) C₁-C₄ linear or branched alkyl;
vi) —C(O)R¹⁹;
   wherein R¹⁹ is chosen from:
   a) C₁-C₄ linear or branched alkyl;
   b) —OR⁹;
      R²⁰ is hydrogen or C₁-C₄ linear alkyl; and
   c) —N(R^{21a})(R^{21b});
   R^{21a} and R^{21b} are each independently hydrogen or C₁-C₄ linear alkyl;
vii) —OC(O)R²²; R²² is C₁-C₄ linear or branched alkyl or phenyl;
viii) —CN;
ix) —NO₂;
x) —SO₂R²³; R²³ is hydrogen, hydroxyl, or C₁-C₄ linear or branched alkyl; and
xi) halogen.

One embodiment of the disclosure relates to compounds having the formula:

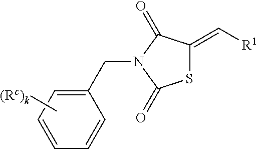

wherein each R^{c} represents from 1 to 5 optionally present organic radicals independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

### R³ Units

The following describes R^{e} units that comprise the compounds suitable for use in treating cancer. R³ units are benzyl or benzyl substituted by from 1 to 5 independently chosen R^{b} units wherein R^{b} units are organic radicals. Non-limiting examples of organic radicals are chosen from:
i) C₁-C₁₂ substituted or unsubstituted linear, branched, or cyclic alkyl; for example, methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), cyclobutyl (C₄), cyclopentyl (C₅), cyclohexyl (C₆);
ii) C₂-C₁₂ substituted or unsubstituted linear, branched, or cyclic alkenyl; for example, ethenyl (C₂), 3-propenyl (C₃), 1-propenyl (also 2-methylethenyl) (C₃), isopropenyl (also 2-methylethen-2-yl) (C₃), buten-4-yl (C₄);
iii) C₂-C₁₂ substituted or unsubstituted linear or branched alkynyl; for example, ethynyl (C₂), prop-2-ynyl (also propargyl) (C₃), propyn-1-yl (C₃);
iv) C₁-C₁₂ substituted or unsubstituted linear or branched haloalkyl; for example, —CH₂F, —CHF₂, —CF₃, —CH₂CH₂F, —CH₂CF₃, and —CF₂CF₃;
iv) C₆ or C₁₀ substituted or unsubstituted aryl; for example, phenyl, naphthyl (also referred to herein as naphthylen-1-yl (C₁₀) or naphthylen-2-yl (C₁₀));
v) C₁-C₉ substituted or unsubstituted heterocyclic; as described herein below;
vi) C₁-C₁₁ substituted or unsubstituted heteroaryl; as described herein below;
vii) —[C(R^{24a})(R^{24b})]_{z}OR²⁵;
   wherein R²⁵ is chosen from:
   a) —H;
   b) C₁-C₁₂ substituted or unsubstituted linear, branched, or cyclic alkyl or C₁-C₁₂ substituted or unsubstituted linear, branched, or cyclic haloalkyl;
   c) C₆ or C₁₀ substituted or unsubstituted aryl or C₇-C₂₀ alkylenearyl;
   d) C₁-C₉ substituted or unsubstituted heterocyclic; and
   e) C₁-C₁₁ substituted or unsubstituted heteroaryl; for example, —OH, —CH₂OH, —OCH₃, —OCF₃, —CH₂OCH₃, —OCH₂CH₃, —CH₂OCH₂CH₃, —OCH₂CH₂CH₃, —CH₂OCH₂CH₂CH₃, —CH₂OC₆H₅, and —CH₂OC₆H₅;
viii) —[C(R^{24a})(R^{24b})]_{z}N(R^{26a})(R^{26b});
   wherein R^{26a} and R^{26b} are each independently chosen from:
   a) —H;
   b) —OR²⁷;
      R²⁷ is hydrogen or C₁-C₄ linear alkyl;
   c) C₁-C₁₂ substituted or unsubstituted linear, branched, or cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; and
   g) R^{26a} and R^{26b} can be taken together to form a substituted or unsubstituted ring having from 3 to 10 carbon atoms and from 0 to 3 heteroatoms chosen from oxygen, nitrogen, and sulfur; for example, —NH₂, —CH₂NH₂, —NHCH₃, —N(CH₃)₂, —NH(CH₂CH₃), —CH₂NHCH₃, —CH₂N(CH₃)₂, —CH₂NH(CH₂CH₃), —NHOH, and —CH₂NHOH;
ix) —[C(R^{24a})(R^{24b})]_{z}C(O)R²⁵;
   wherein R²⁸ is chosen from:
   a) C₁-C₁₂ substituted or unsubstituted linear, branched, or cyclic alkyl;
   b) —OR²⁹;
      R²⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
   c) —N(R^{30a})(R^{30b});
      R^{30a} and R^{30b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{30a} and R^{30b} can be taken together to form a substituted or unsubstituted ring having from 3 to 10 carbon atoms and from 0 to 3 heteroatoms chosen from oxygen, nitrogen, and sulfur;
      for example, —COCH₃, —CO₂H, —CO₂CH₃, —CONH₂, —CH₂COCH₃, —CH₂CO₂H, —CH₂CO₂CH₃, —CH₂CONH₂, —CONHCH₃, —CO₂CH₂CH₃, —CH₂CONHCH₃, and —CON(CH₃)₂;
x) —[C(R^{24a})(R^{24b})]_{z}OC(O)R³¹;
   wherein R³¹ is chosen from:
   a) C₁-C₁₂ substituted or unsubstituted linear, branched, or cyclic alkyl; and
b) —N(R^{32a})(R^{32b});
   R^{32a} and R^{32b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{32a} and R^{32b} can be taken together to form a substituted or unsubstituted ring having from 3 to 10 carbon atoms and from 0 to 3 heteroatoms chosen from oxygen, nitrogen, and sulfur;
   for example, —OC(O)CH₃, —OC(O)CH₂CH₃, —CH₂OC(O)CH₃, —OC(O)NH₂, —CH₂OC(O)NH₂, and —CH₂OC(O)N(CH₃)₂;
xi) —[C(R^{24a})(R^{24b})]_{z}NR³³C(O)R³⁴
   wherein R³³ is chosen from:
   a) —H; and
   b) C₁-C₄ substituted or unsubstituted linear, branched, or cyclic alkyl;
   wherein R³⁴ is chosen from:
   a) C₁-C₁₂ substituted or unsubstituted linear, branched, or cyclic alkyl; and
   b) —N(R^{35a})(R^{35b});
      R^{35a} and R^{35b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{35a} and R^{35b} can be taken together to form a substituted or unsubstituted ring having from 3 to 10 carbon atoms and from 0 to 3 heteroatoms chosen from oxygen, nitrogen, and sulfur;
      for example, —NHC(O)CH₃, —NHC(O)CH₂CH₃, —CH₂NHC(O)CH₃, —NHC(O)NH₂, and —CH₂NHC(O)N(CH₃)₂;
xii) —[C(R^{24a})(R^{24b})]_{z}CN;
xiii) —[C(R^{24a})(R^{24b})]_{z}NO₂;
xiv) —[C(R^{24a})(R^{24b})]_{z}SO₂R³⁶;
   R³⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
   for example, —SO₂H, —CH₂SO₂H, —SO₂CH₃, —CH₂SO₂CH₃, —SO₂C₆H₅, and —CH₂SO₂C₆H₅; and
xv) halogen; —F, —Cl, —Br, and —I;
   R^{24a} and R^{24b} are each independently hydrogen or C₁-C₄ alkyl.

The index z can have any value from 0 to 6, for example, z can be 0, 1, 2, 3, 4, 5, or 6.

A first embodiment of the disclosure relates to compounds wherein R³ is phenyl.

Another embodiment of the disclosure relates to compounds wherein R³ units are benzyl units substituted by from 1 to 5 independently chosen R^{b} units wherein R^{b} units are organic radicals chosen from:
i) C₁-C₄ linear, branched, or cyclic alkyl;
ii) C₁-C₄ haloalkyl;
iii) phenyl;
iv) —OR²⁵;
   wherein R²⁵ is chosen from:
   a) —H; and
   b) C₁-C₄ linear or branched alkyl or C₁-C₄ linear or branched haloalkyl;
v) —N(R^{26a})(R^{26b});
   wherein R^{26a} and R^{26b} are each independently chosen from:
   a) —H; and
   b) C₁-C₄ linear or branched alkyl;
vi) —C(O)R²⁸;
   wherein R²⁸ is chosen from:
   a) C₁-C₄ linear or branched alkyl;
   b) —OR²⁸;
      R²⁸ is hydrogen or C₁-C₄ linear alkyl; and
c)—N(R^{30a})(R^{30b});
   R^{30a} and R^{30b} are each independently hydrogen or C₁-C₄ linear alkyl;
vii) —OC(O)R³¹; R³¹ is C₁-C₄ linear or branched alkyl or phenyl;
viii) —CN;
ix) —NO₂;
x) —SO₂R³⁶; R³⁶ is hydrogen, hydroxyl, or C₁-C₄ linear or branched alkyl; and
xi) halogen.

One iteration of this embodiment relates to compounds having the formula:

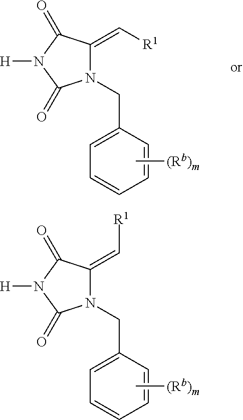

wherein the index m is from 1 to 5, R¹ is the same as defined herein above, and each R^{b} is an organic radical independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

One example of this iteration relates to compounds wherein R^{b} is C₁-C₄ linear, branched, or cyclic alkyl, for example, the compounds having the formulae:

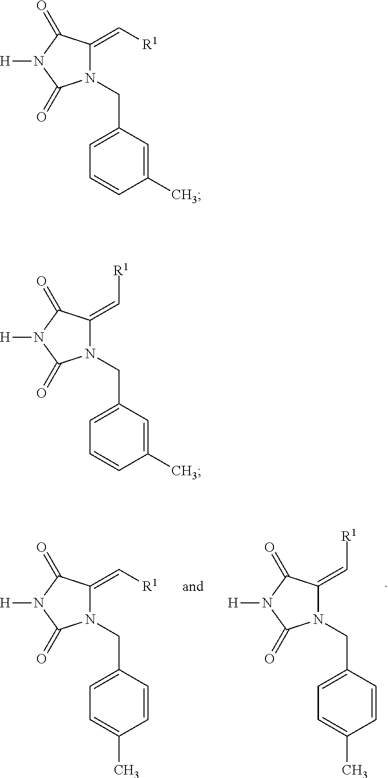

Another example of this iteration relates to compounds wherein R^{b} is C₁-C₄ linear, branched, or cyclic haloalkyl, for example, the compounds having the formulae:

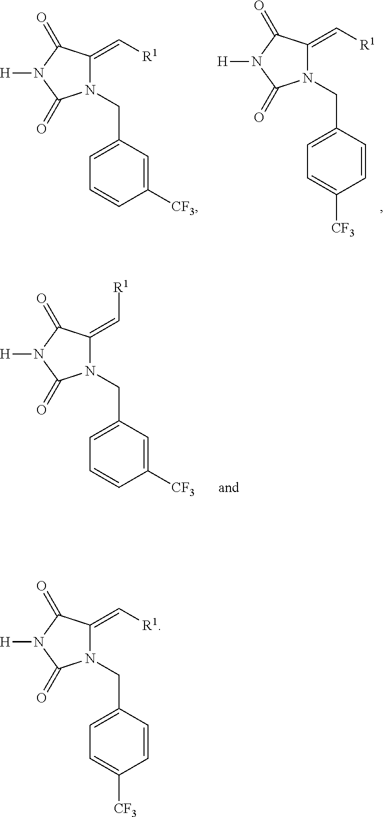

A further example of this iteration relates to compounds wherein R^{b} is C₁-C₄ linear, branched, or cyclic alkoxy, for example, the compounds having the formulae:

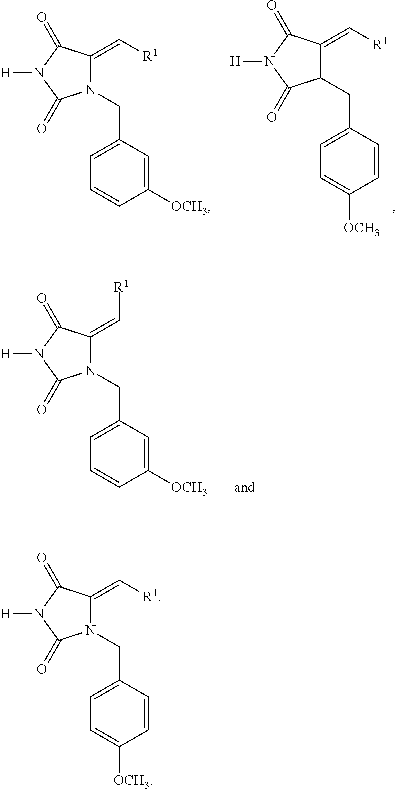

### COMPOSITIONS

A first embodiment of the disclosure relates to compounds having the formula:

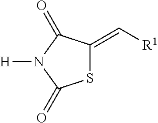

wherein R¹ represents from 1 to 5 substitutions for hydrogen. Table I provides non-limiting examples of compounds according to the present disclosure.

TABLE I

| No. | R¹ |
|---|---|
| 1 | 2-fluorophenyl |
| 2 | 3-fluorophenyl |
| 3 | 4-fluorophenyl |
| 4 | 2-chlorophenyl |
| 5 | 3-chlorophenyl |
| 6 | 4-chlorophenyl |
| 7 | 2-bromophenyl |
| 8 | 3-bromophenyl |
| 9 | 4-bromophenyl |
| 10 | 2-methylphenyl |
| 11 | 3-methylphenyl |
| 12 | 4-methylphenyl |
| 13 | 2-ethylphenyl |
| 14 | 3-ethylphenyl |
| 15 | 4-ethylphenyl |
| 16 | 2-methoxyphenyl |
| 17 | 3-methoxyphenyl |
| 18 | 4-methoxyphenyl |
| 19 | 2-ethoxyphenyl |
| 20 | 3-ethoxyphenyl |
| 21 | 4-ethoxyphenyl |
| 22 | 2-trifluoromethylphenyl |
| 23 | 3-trifluoromethylphenyl |
| 24 | 4-trifluoromethylphenyl |
| 25 | 2-trifluoromethoxyphenyl |
| 26 | 3-trifluoromethoxyphenyl |
| 27 | 4-trifluoromethoxyphenyl |
| 28 | 2-(1,1,2,2-tetrafluoroethyl) phenyl |
| 29 | 3-(1,1,2,2-tetrafluoroethyl) phenyl |
| 30 | 4-(1,1,2,2-tetrafluoroethyl) phenyl |
| 31 | 2-(dimethylamino)phenyl |
| 32 | 3-(dimethylamino)phenyl |
| 33 | 4-(dimethylamino)phenyl |
| 34 | 2-(diethylamino)phenyl |
| 35 | 3-(diethylamino)phenyl |
| 36 | 4-(diethylamino)phenyl |
| 37 | 2-propylphenyl |
| 38 | 3-propylphenyl |
| 39 | 4-propylphenyl |
| 40 | 2-iso-propylphenyl |
| 41 | 3-iso-propylphenyl |
| 42 | 4-iso-propylphenyl |

The compounds according to this embodiment can be prepared by the procedure outlined in Scheme I and described in Example I.

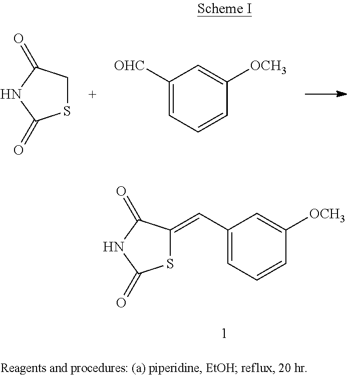

### Example 1

### (Z)-5-(3-Methoxybenzylidene)thiazolidine-24-dione (1)

Preparation of (Z)-5-(3-methoxybenzylidene)thiazolidine-24-dione (1): 2,4-thiazolidinedione (117 mg, 1 mmol) was dissolved in ethanol (8 mL), followed by addition of m-anisaldehyde (122 uL, 1 mmol) and piperidine (79 uL, 0.8 mmol). The mixture was refluxed for 20 hours. The yellow solution was poured into water (˜60 mL) and a yellow precipitate forms. The mixture was acidified with acetic acid to a pH of 3-4 and the reaction mixture placed in the cold overnight. The resulting precipitate is collected by filtration to afford 115 mg (49% yield) of the desired product as a yellow solid. ¹H NMR (DMSO) δ 7.77 (s, 1H), 7.45 (dd, J₁=J₂=8.0 Hz, 1H), 7.16 (d, J=8.0 Hz, 1H), 7.15 (s, 1H), 7.07 (d, J=8.0 Hz, 1H), 3.81 (s, 3H); ¹³C NMR (DMSO) δ 168.2, 167.7, 160.1, 134.9, 132.2, 130.9, 124.4, 122.3, 116.8, 115.8, 55.7.

The following are non-limiting examples of compounds according to the first embodiment of the present disclosure:

(Z)-5-(4-Chlorobenzylidene)thiazolidine-2,4-dione: ¹H NMR (DMSO) δ 7.79 (s, 1H), 7.61 (m, 4H); ¹³C NMR (DMSO) δ 167.8, 167.4, 135.1, 132.1, 131.8 (2C), 130.5, 129.5 (2C), 124.6.

(Z)-5-(3-Chlorobenzylidene)thiazolidine-2,4-dione: ¹H NMR (DMSO) δ 7.79 (s, 1H), 7.68 (s, 1H), 7.52-7.60 (m, 3H); ¹³C NMR (DMSO) δ 167.8, 167.4, 135.5, 134.1, 131.3, 130.3, 130.2, 130.1, 128.0, 125.7.

(Z)-5-(4-Trifluoromethylbenzylidene)thiazolidine-2,4-dione: ¹H NMR (DMSO) 7.89 (d, J=8.4 Hz, 2H), 7.87 (s, 1H), 7.81 (d, J=8.4H, 2H); ¹³C NMR (DMSO) δ 167.7, 167.3, 137.2, 130.6 (2C), 130.0, 129.7, 126.9, 126.2 (t, J=3.8 Hz, 2C), 120.4 (t, J=271 Hz, 1C).

(Z)-5-(3-Trifluoromethylbenzylidene)thiazolidine-2,4-dione: ¹H NMR (CDCl₃) δ 7.88 (s, 1H), 7.75 (s, 1H), 7.70 (d, J=7.6 Hz, 1H), 7.68 (d, J=7.6H, 1H), 7.63 (J₁=J₂=7.6 Hz, 1H); ¹³C NMR (CDCl₃) δ 166.5, 166.3, 133.8, 132.7, 132.5, 132.0 (t, J=32.6 Hz, 1C), 129.9, 127.1 (t, J=3.8 Hz, 1C), 127.0 (t, J=3.8 Hz, 1C), 124.6, 123.5 (t, J=271 Hz, 1C).

(Z)-5-(4-Trifluoromethoxybenzylidene)thiazolidine-2,4-dione: ¹H NMR (CDCl₃) δ 7.85 (s, 1H), 7.55 (d, J=8.4 Hz, 2H), 7.32 (d, J=8.4H, 2H); ¹³C NMR (CDCl₃) δ 167.0, 166.8, 150.6 (t, J=2.2 Hz, 1H), 132.8, 131.9 (2C), 131.4, 123.3, 121.4 (2C), 120.4 (t, J=257 Hz, 1C).

(Z)-5-(4-Ethylbenzylidene)thiazolidine-2,4-dione: ¹H NMR (DMSO) δ 7.76 (s, 1 H), 7.51 (d, J=8.4 Hz, 2H), 7.38 (d, J=8.4 Hz, 2H), 2.66 (q, d=7.6 Hz, 2H), 1.20 (t, J=7.6 Hz, 3H); ¹³C NMR (DMSO) δ 168.1, 167.6, 147.0, 132.0, 130.7, 130.4 (2C), 128.9 (2C), 122.6, 28.3, 15.4.

(Z)-5-(4-Dimethylaminobenzylidene)thiazolidine-2,4-dione: ¹H NMR (DMSO) δ 7.66 (s, 1H), 7.44 (d, J=8.8 Hz, 2H), 6.82 (d, J=8.8 Hz, 2H), 3.32 (s, 6H); ¹³C NMR (DMSO) δ 168.4, 167.8, 151.6, 133.1, 132.3 (2C), 120.0, 115.9, 112.2 (2C), 39.5 (2C).

(Z)-5-(4-Fluorobenzylidene)thiazolidine-2,4-dione: R_{f}=0.27 (3% methanol in chloroform). ¹H NMR (DMSO) δ 7.81 (s, 1H), 7.67 (dd, J=8.8 Hz, J₂=5.6 Hz, 2H), 7.39 (dd, J₁=J₂=8.8 Hz, 2H); ¹³C NMR (DMSO) δ 168.0, 167.6, 163.0 (d, J=249 Hz, 1C), 132.6 (d, J=8.4 Hz, 2C), 130.8, 129.9 (d, J=3 Hz, 1C), 123.6 (d, J=2.2 Hz, 1C), 116.7 (d, J=22 Hz, 2C).

(Z)-5-(4-Methylbenzylidene)thiazolidine-2,4-dione: ¹H NMR (DMSO) δ 7.76 (s, 1H), 7.49 (d, J=8.0 Hz, 2H), 7.35 (d, J=8.0 Hz, 2H), 2.36 (s, 3H); ¹³C NMR (DMSO) δ 168.1, 167.6, 140.9, 132.0, 130.5, 130.2 (2C), 130.1 (2C), 122.5, 21.2.

(Z)-5-(3-Trifluoromethoxybenzylidene)thiazolidine-2,4-dione: R_{f}=0.27 (3% methanol in chloroform). ¹H NMR (CDCl₃) δ 7.88 (bs, 1H), 7.83 (s, 1H), 7.52 (dd, J₁=J₂=8.0 Hz, 1H), 7.43 (d, J=8.0 Hz, 1H), 7.34 (s, 1H), 7.29 (d, J=8.0 Hz, 1H); ¹³C NMR (CDCl₃) δ 167.5, 167.3, 150.0 (m, 1C), 135.1, 132.8, 130.9, 128.4, 124.8, 123.0, 122.5, 120.6 (q, J=257.3 Hz, 1C).

(Z)-5-[3-(1,1,2,2-Tetrafluoroethoxy)benzylidene]thiazolidine-2,4-dione: R_{f}=0.23 (3% methanol in chloroform). ¹H NMR (DMSO) δ 12.70 (bs, 1H), 7.85 (s, 1H), 7.65 (dd, J₁=J₂=8.0 Hz, 1H), 7.59 (d, J=8.0 Hz, 1H), 7.52 (s, 1H), 7.40 (dd, J₁=8.0 Hz, J₂=1.2 Hz, 1H), 6.84 (tt, J₁=52 Hz, J₂=3.2 Hz, 1H); ¹³C NMR (DMSO) δ 167.6, 167.3, 148.7, 135.4, 131.3, 130.3, 128.1, 125.7, 123.3, 122.9, 116.6 (tt, J₁=270 Hz, J₂=28.1 Hz, 1C), 107.9 (tt, J₁=247 Hz, J₂=40.2 Hz, 1C).

(Z)-5-(4-Bromobenzylidene)thiazolidine-2,4-dione: ¹H NMR (DMSO) δ 7.76 (s, 1H), 7.73 (d, J=8.4 Hz, 2H), 7.54 (d, J=8.4 Hz, 2H); ¹³C NMR (DMSO) δ 168.0, 167.8, 132.6, 132.5 (2C), 131.9 (2C), 131.4, 130.5, 124.0.

(Z)-5-(4-iso-Propylbenzylidene)thiazolidine-2,4-dione: ¹H NMR (CDCl₃) δ 7.87 (s, 1H), 7.44 (d, J=8.0 Hz, 2H), 7.34 (d, J=8.0 Hz, 2H), 2.96 (tt, J₁=J₂=6.8 Hz, 1H), 1.27 (d, J=6.8 Hz, 6H); ¹³C NMR (CDCl₃) δ 167.8, 167.4, 152.6, 135.0, 130.8 (2C), 130.7, 127.7 (2C), 121.3, 34.4, 23.9 (2C).

(Z)-5-(3-Fluorobenzylidene)thiazolidine-2,4-dione: ¹H NMR (DMSO) δ 12.68 (bs, 1 H), 7.80 (s, 1H), 7.59 (m, 1H), 7.42-7.48 (m, 2H), 7.34 (ddd, J₁=J₂=8.0 Hz, J₃=2 Hz, 1H); ¹³C NMR (DMSO) δ 167.9, 167.6, 162.4 (d, J=243.6 Hz, 1C), 135.7 (d, J=8.3 Hz, 1C), 131.5 (d, J=8.3 Hz, 1C), 130.3, 125.7 (2C), 117.3 (d, J=21.2 Hz, 1C), 116.8 (d, J=22.8 Hz, 1C).

(Z)-5-(4-Methoxybenzylidene)thiazolidine-2,4-dione: ¹H NMR (DMSO) δ 7.74 (s, 1 H), 7.56 (d, J=8.8 Hz, 2H), 7.10 (d, J=8.8 Hz, 2H), 3.83 (s, 3H); ¹³C NMR (DMSO) δ 168.4, 168.1, 160.9, 132.0 (2C), 131.6, 125.6, 114.9 (2C), 55.5.

(Z)-5-(4-Ethoxybenzylidene)thiazolidine-2,4-dione: ¹H NMR (DMSO) δ 12.50 (bs, 1H), 7.73 (s, 1H), 7.54 (d, J=8.6 Hz, 2H), 7.08 (d, J=8.6 Hz, 2H), 4.10 (q, J=6.8 Hz, 2H), 1.35 (t, J=6.8 Hz, 3H); ¹³C NMR (DMSO) δ 168.5, 168.3, 160.4, 132.2 (2C), 131.6, 125.6, 121.0, 115.4 (2C), 63.7, 14.7.

(Z)-5-(3-Methylbenzylidene)thiazolidine-2,4-dione: ¹H NMR (DMSO) δ 12.59 (bs, 1H), 7.75 (s, 1 FI), 7.37-7.45 (m, 3H), 7.30 (d, J=7.2 Hz, 1H), 2.37 (s, 3H); ¹³C NMR (DMSO) δ 168.1, 167.5, 138.8, 133.2, 132.0, 131.3, 130.7, 129.4, 127.2, 123.5, 21.1.

(Z)-5-(4-Propoxybenzylidene)thiazolidine-2,4-dione: ¹H NMR (DMSO) δ 12.50 (bs, 1H), 7.75 (s, 1H), 7.54 (d, J=8.8 Hz, 2H), 7.09 (d, J=8.8 Hz, 2H), 4.01 (t, J=6.8 Hz, 2H), 1.75 (m, 2H), 0.98 (t, J=7.6 Hz, 3H); ¹³C NMR (DMSO) δ 168.1, 167.6, 160.7, 132.3 (2C), 132.1, 125.5, 120.3, 115.5 (2C), 69.5, 22.1, 10.5.

(Z)-5-(3-Bromobenzylidene)thiazolidine-2,4-dione: ¹H NMR (DMSO) δ 12.69 (bs, 1H), 7.82 (dd, J₁=J₂=2.0 Hz, 1H), 7.78 (s, 1H), 7.68 (ddd, J=8.0 Hz, J₂=2.0 Hz, J₃=0.8 Hz, 1H), 7.58 (d, J=8.0 Hz, 1H), 7.50 (dd, J₁=J₂=8.0 Hz, 1H); ¹³C NMR (DMSO) δ 167.7, 167.3, 135.7, 133.0, 132.9, 131.5, 130.2, 128.3, 125.6, 122.6.

A further embodiment of the disclosure relates to compounds having the formula:

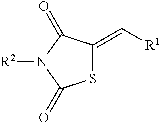

wherein non-limiting examples of R¹ and R² are provided herein below in Table II.

TABLE II

| No. | R¹ | R² |
|---|---|---|
| 43 | 3-fluorophenyl | benzyl |
| 44 | 4-fluorophenyl | benzyl |
| 45 | 3-chlorophenyl | benzyl |
| 46 | 4-chlorophenyl | benzyl |
| 47 | 3-bromophenyl | benzyl |
| 48 | 4-bromophenyl | benzyl |
| 49 | 3-methylphenyl | benzyl |
| 50 | 4-methylphenyl | benzyl |
| 51 | 3-ethylphenyl | benzyl |
| 52 | 4-ethylphenyl | benzyl |
| 53 | 3-methoxyphenyl | benzyl |
| 54 | 4-methoxyphenyl | benzyl |
| 55 | 3-trifluoromethylphenyl | benzyl |
| 56 | 4-trifluoromethylphenyl | benzyl |
| 57 | 3-trifluoromethoxyphenyl | benzyl |
| 58 | 4-trifluoromethoxyphenyl | benzyl |
| 59 | 3-fluorophenyl | 3-trifluoromethoxybenzyl |
| 60 | 4-fluorophenyl | 3-trifluoromethoxybenzyl |
| 61 | 3-chlorophenyl | 3-trifluoromethoxybenzyl |
| 62 | 4-chlorophenyl | 3-trifluoromethoxybenzyl |
| 63 | 3-bromophenyl | 3-trifluoromethoxybenzyl |
| 64 | 4-bromophenyl | 3-trifluoromethoxybenzyl |
| 65 | 3-methylphenyl | 3-trifluoromethoxybenzyl |
| 66 | 4-methylphenyl | 3-trifluoromethoxybenzyl |
| 67 | 3-ethylphenyl | 3-trifluoromethoxybenzyl |
| 68 | 4-ethylphenyl | 3-trifluoromethoxybenzyl |
| 69 | 3-methoxyphenyl | 3-trifluoromethoxybenzyl |
| 70 | 4-methoxyphenyl | 3-trifluoromethoxybenzyl |
| 71 | 3-trifluoromethylphenyl | 3-trifluoromethoxybenzyl |
| 72 | 4-trifluoromethylphenyl | 3-trifluoromethoxybenzyl |
| 73 | 3-trifluoromethoxyphenyl | 3-trifluoromethoxybenzyl |
| 74 | 4-trifluoromethoxyphenyl | 3-trifluoromethoxybenzyl |

The compounds encompassed by this embodiment of the disclosure can be made by the procedure outlined herein below in Scheme II and described in Example 2.

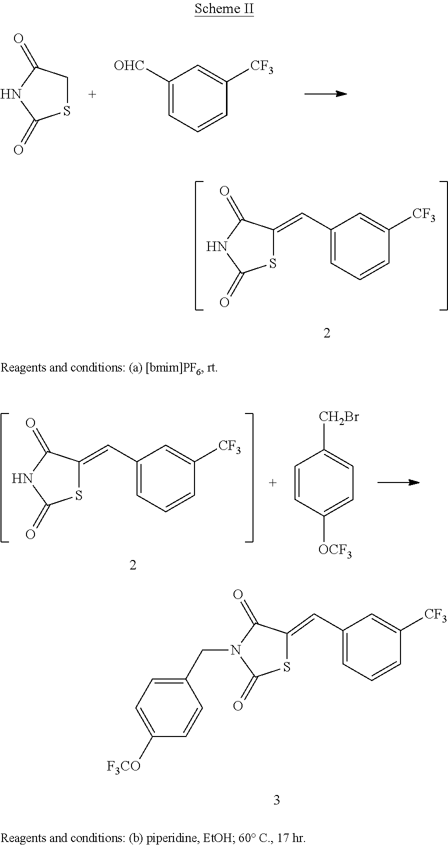

### Example 2

### (Z)-3-(4-Trifluoromethoxybenzyl)-5-(3-trifluoromethylbenzylidene)-thiazolidine-2,4-dione (3)

Preparation in situ of 5-(3-trifluoromethylbenzylidene)thiazolidine-2,4-dione (2) and (Z)-3-(4-trifluoromethoxybenzyl)-5-(3-trifluoromethylbenzylidene)-thiazolidine-2,4-dione (4): A flask is charged with 2,4-thiazolidinedione (59 mg, 0.5 mmol), 3-trifluoromethylbenzaldehyde (67 μL, 87 mg, 0.5 mmol) and 1-butyl-3-methyl-imidazolium hexafluorophosphate [bmim]PF₆ (2 mL), followed by addition of Et₃N (84 μL, 0.6 mmol) and 4-trifluoromethoxybenzyl bromide (0.6 mmol, 96 μL). The mixture was stirred at 60° C. for 17 hours. After cooling down to room temperature, the mixture was extracted with ether (4×15 mL). The collected ether was checked by TLC (25% ethyl acetate in hexane, silica) and one major IN positive spot: R_{f}=0.28 was found. After filtration and concentration, the residue was purified on a CHROMATOTRON™ (silica plate) eluting with a gradient of 15%-25% ethyl acetate in hexane to afford 5 mg (1% yield) of a semisolid product. ¹H NMR (CDCl₃) δ 7.92 (s, 1H), 7.73 (s, 1H), 7.59-7.70 (m, 3H), 7.48 (d, J=8.0 Hz, 2H), 7.19 (d, J=8.0 Hz, 2H), 4.90 (s, 2H); ¹³C NMR (CDCl₃) δ 167.3, 165.9, 149.5, 134.2, 133.8, 132.8, 132.5, 132.1 (q, J=33 Hz, 1C), 130.8 (2C), 130.1, 127.0-127.2 (m, 2C), 123.8, 123.7 (q, J=271 Hz, 1C), 121.5 (2C), 120.6 (q, J=256 Hz, 1C), 44.8.

The following are further examples of this embodiment of the disclosure:

3-(2-Fluorobenzyl)-5-(4-methoxybenzylidene)thiazolidine-2,4-dione: ¹H NMR (CDCl₃) δ 7.24-7.32 (m, 2H), 7.00-7.11 (m, 2H), 4.85 (s, 2H), 3.97 (s, 2H); ¹³C NMR (CDCl₃) δ 171.4, 171.1, 160.8 (d, J=247 Hz, 1C), 130.5 (d, J=3.0 Hz, 1C), 130.2 (d, J=8.3 Hz, 1C), 124.4 (d, J=3.8 Hz, 1C), 122.0 (d, J=14 Hz, 1C), 115.8 (d, J=21 Hz, 1C), 39.3 (d, J=4.6 Hz, 1C), 33.9.

3-(2-Fluorobenzyl)-5-(4-fluorobenzylidene)thiazolidine-2,4-dione: ¹H NMR (CDCl₃) δ 7.88 (s, 1H), 7.50 (dd, J1=8.4 Hz, 2H), 7.27-7.37 (m, 2H), 7.17 (dd, J₁=J₂=8.4 Hz, 2H), 7.04-7.14 (m, 2H), 5.00 (s, 2H); ¹³C NMR (CDCl₃) δ 167.4, 166.1, 163.9 (d, J=253 Hz, 1C), 160.9 (d, J=247 Hz, 1C), 133.2, 132.5 (d, J=8.4 Hz, 2C), 130.4 (d, J=3.8 Hz, 1C), 130.2 (d, J=7.6 Hz, 1C), 129.7 (d, J=3.8 Hz, 1C), 124.5 (d, J=3.8 Hz, 1C), 122.0 (d, J=14.4 Hz, 1C), 121.1 (d, J=2.3 Hz, 1C), 116.8 (d, J=22.0 Hz, 2C), 115.9 (d, J=21.2 Hz, 1C), 39.3 (d, J=4.5 Hz, 1C).

Another embodiment of the disclosure relates to compounds having the formula:

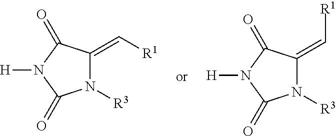

wherein the compounds can be present as either the (Z)-isomer alone, the (E)-isomer alone, or as a mixture of the (Z)- and (E)-isomers. Non-limiting examples of R¹ and R³ are provided herein below in Table III.

TABLE III

| No. | R¹ | R³ |
|---|---|---|
| 75 | 3-fluorophenyl | 3-trifluoromethylbenzyl |
| 76 | 4-fluorophenyl | 3-trifluoromethylbenzyl |
| 77 | 3-chlorophenyl | 3-trifluoromethylbenzyl |
| 78 | 4-chlorophenyl | 3-trifluoromethylbenzyl |
| 79 | 3-bromophenyl | 3-trifluoromethylbenzyl |
| 80 | 4-bromophenyl | 3-trifluoromethylbenzyl |
| 81 | 3-methylphenyl | 3-trifluoromethylbenzyl |
| 82 | 4-methylphenyl | 3-trifluoromethylbenzyl |
| 83 | 3-ethylphenyl | 3-trifluoromethylbenzyl |
| 84 | 4-ethylphenyl | 3-trifluoromethylbenzyl |
| 85 | 3-methoxyphenyl | 3-trifluoromethylbenzyl |
| 86 | 4-methoxyphenyl | 3-trifluoromethylbenzyl |
| 87 | 3-trifluoromethylphenyl | 3-trifluoromethylbenzyl |
| 88 | 4-trifluoromethylphenyl | 3-trifluoromethylbenzyl |
| 89 | 3-trifluoromethoxyphenyl | 3-trifluoromethylbenzyl |
| 90 | 4-trifluoromethoxyphenyl | 3-trifluoromethylbenzyl |
| 91 | 3-fluorophenyl | benzyl |
| 92 | 4-fluorophenyl | benzyl |
| 93 | 3-chlorophenyl | benzyl |
| 94 | 4-chlorophenyl | benzyl |
| 95 | 3-bromophenyl | benzyl |
| 96 | 4-bromophenyl | benzyl |
| 97 | 3-methylphenyl | benzyl |
| 98 | 4-methylphenyl | benzyl |
| 99 | 3-ethylphenyl | benzyl |
| 100 | 4-ethylphenyl | benzyl |
| 101 | 3-methoxyphenyl | benzyl |
| 102 | 4-methoxyphenyl | benzyl |
| 103 | 3-trifluoromethylphenyl | benzyl |
| 104 | 4-trifluoromethylphenyl | benzyl |
| 105 | 3-trifluoromethoxyphenyl | benzyl |
| 106 | 4-trifluoromethoxyphenyl | benzyl |

The compounds encompassed by this embodiment of the disclosure can be made by the procedure outlined herein below in Schemes III and IV and described in Examples 3 and 4.

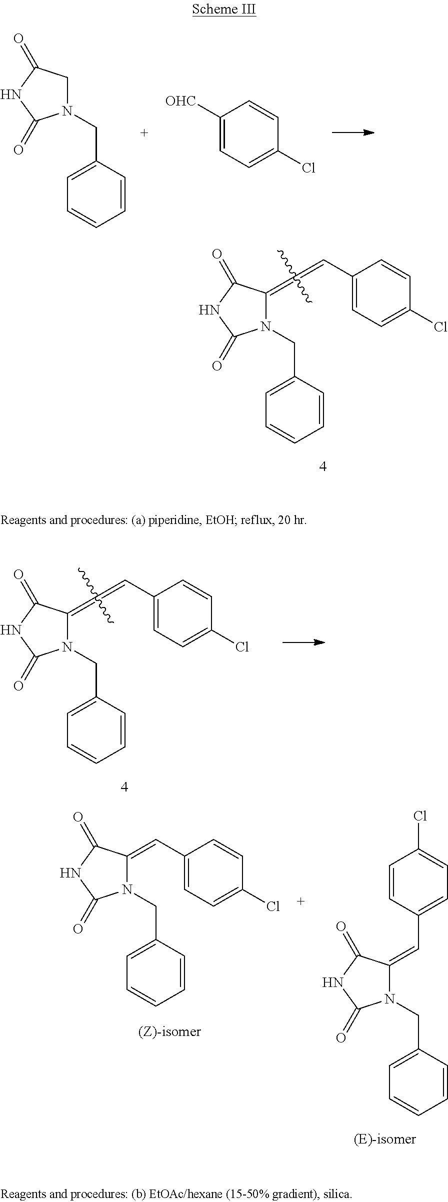

### Example 3

### (E) and (Z)-1-Benzyl-5-(4-chlorobenzylidene)imidazolidine-2,4-dione (4)

Preparation of 1-benzyl-5-(4-chlorobenzylidene)imidazolidine-2,4-dione (4): 1-Benzylhydantoin (190 mg, 1 mmol) and p-chlorobenzaldehyde (142 mg, 1 mmol) were dissolved in ethanol (7 mL), followed by addition of piperidine (79 uL, 0.8 mmol). The mixture was refluxed for 24 hours. The yellowish solution was poured into water (˜60 mL). The mixture was acidified with acetic acid to pH 5, then extracted with chloroform (3×30 mL). The collected organic solution was dried over sodium sulfate and filtered through a pad of silica eluting with 25% ethyl acetate in hexane then concentrated to afford a crude admixture.

Separation and purification of (E) and (Z) isomers of (4): The crude admixture was purified using a CHROMATOTRON™ eluting with a 15% to 50% gradient of ethyl acetate in hexane to afford three components: (A) Rf=0.42 (25% ethyl acetate in hexane); (B) Rf=0.14 (25% ethyl acetate in hexane); (C) 0.20 (50% ethyl acetate in hexane). Based on NMR spectra, (B) is the product (20 mg, 6% yield, E/Z≈9/1). (E)-isomer is the major component: ¹H NMR (DMSO) δ 8.87 (bs, 1H), 7.27-7.40 (m, 7H), 6.14 (s, 1H), 4.91 (s, 2H); ¹³C NMR (DMSO) δ 162.0, 153.4, 135.2, 135.0, 131.8 (2C), 130.7, 129.3 (2C), 128.8, 128.6 (2C), 128.3, 127.2 (2C), 117.9, 43.8. Z-isomer is the minor component: ¹H NMR (DMSO) δ 7.25-7.40 (m, 1H), 7.24 (d, J=8.0 Hz, 2H), 7.14 (d, J=8.0 Hz, 2H), 6.97 (d, J=8.0 Hz, 2H), 6.80 (s, 1H), 6.61 (dd, J₁=8.0 Hz, J₂=1.6 Hz, 2H), 4.73 (s, 2H).

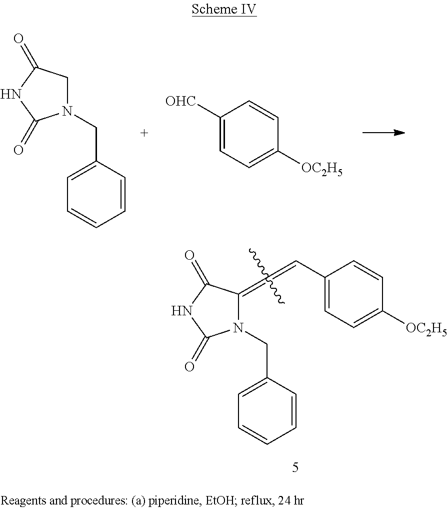

### Example 4

### (Z/E)-1-Benzyl-5-(4-ethoxybenzylidene)imidazolidine-2,4-dione

Preparation of (Z/E)-1-Benzyl-5-(4-ethoxybenzylidene)imidazolidine-2,4-dione (5): To a flask is charged 1-benzylhydantoin (190 mg, 1 mmol), 4-ethoxybenzaldehyde (139 uL, 150 mg, 1 mmol) and ethanol (7 mL), followed by addition of piperidine (100 uL, 1 mmol). The mixture was refluxed for 24 hours. The yellowish solution was poured into water (˜60 mL) and a precipitate formed. The solution was acidified with acetic acid and additional precipitate formed. The reaction vessel was kept in the cold overnight and the resulting precipitate was collected by filtration. The solid was washed with water and dried in the atmosphere. The mixture was purified using a CHROMATOTRON™ eluting with hexane-CHCl₃-1% methanol-3% methanol in chloroform to afford 136 mg (50% yield) of a yellowish solid. Rf=0.21 (3% methanol in chloroform, silica). NMR indicates a mixture of Z- (minor) and E- (major) isomers (approximately 1:4). The major E-compound: ¹H NMR (CDCl₃) δ 8.94 (bs, 1H), 7.80 (d, J=8.8 Hz, 2H), 7.28-7.38 (m, 5H), 6.84 (d, J=8.8 Hz, 2H), 6.17 (s, 1H), 4.91 (s, 2H), 4.04 (q, d=6.8 Hz, 2H), 1.40 (t, J=6.8 Hz, 3H); ¹³C NMR (CDCl₃) δ 162.4, 160.1, 153.5, 135.4, 132.6 (2C), 129.2 (2C), 128.1, 127.2 (2C), 126.6, 124.8, 120.1, 114.4 (2C), 63.7, 43.7, 14.9. The minor Z-compound: ¹H NMR (CDCl₃) δ 9.02 (bs, 1H), 7.13 (d, J=8.4 Hz, 2H), 7.05 (d, J=8.4 Hz, 2H), 6.81-6.86 (m, 4H), 6.67 (dd, J₁=8.0 Hz, J₂=2.0 Hz, 2H), 4.79 (s, 2H), 4.06 (q, J=7.2 Hz, 2H), 1.45 (t, J=7.2 Hz, 3H); ¹³C NMR (CDCl₃) δ 162.3, 159.5, 155.7, 135.4, 131.2 (2C), 128.6 (2C), 127.9, 127.7 (2C), 124.8, 114.7, 114.4 (2C), 63.8, 45.2, 14.9.

The following are further non-limiting examples of this iteration of the disclosed compounds:

(E/Z)-1-Benzyl-5-(4-methoxybenzylidene)imidazolidine-2,4-dione: E-isomer: ¹H NMR (CDCl₃) δ 7.79 (d, J=8.8 Hz, 2H), 7.27-7.39 (m, 5H), 6.85 (d, J=8.8 Hz, 2H), 6.17 (s, 1H), 4.91 (s, 2H), 3.81 (s, 3H); ¹³C NMR (CDCl₃) δ 162.0, 160.7, 153.1, 135.4, 132.5 (2C), 129.2 (2C), 128.2, 127.2 (2C), 126.8, 125.1, 119.9, 113.9 (2C), 55.5, 43.8.

(E)-1-Benzyl-5-(4-bromobenzylidene)imidazolidine-2,4-dione ¹H NMR (CDCl₃) δ 7.60 (d, J=8.4 Hz, 2H), 7.44 (d, J=8.4 Hz, 2H), 7.28-7.40 (m, 5H), 6.12 (s, 1H), 4.91 (s, 2H); ¹³C NMR (CDCl₃) δ 161.5, 152.8, 135.0, 132.0 (2C), 131.6 (2C), 131.1, 129.3 (2C), 128.9, 128.3, 127.2 (2C), 123.6, 117.9, 43.8.

(E)-1-Benzyl-5-(4-dimethylaminobenzylidene)imidazolidine-2,4-dione: ¹H NMR (DMSO) δ 11.35 (bs, 1H), 7.87 (d, J=9.2 Hz, 2H), 7.24-7.40 (m, 5H), 7.65 (d, J=9.2 Hz, 2H), 6.28 (s, 1H), 4.87 (s, 2H), 2.94 (s, 6H); ¹³C NMR (DMSO) δ 162.4, 152.9, 149.9, 136.1, 131.5 (2C), 128.2 (2C), 126.8, 126.5 (2C), 124.2, 119.8, 118.3, 110.7 (2C), 41.4, 39.2 (2C).

(E/Z)-1-Benzyl-5-(2-methoxybenzylidene)imidazolidine-2,4-dione: E-isomer=¹H NMR (CDCl₃) δ 9.15 (bs, 1H), 7.62 (m, 1H), 7.11-7.39 (m, 7H), 6.85-6.88 (m, 1H), 6.19 (s, 1H), 4.91 (s, 2H), 3.82 (s, 3H); ¹³C NMR (CDCl₃) δ 162.2, 159.5, 153.7, 135.1, 133.5, 129.3, 129.2 (2C), 128.2, 127.2 (2C), 126.9, 123.6, 119.6, 116.0, 114.9, 55.5, 43.7.

### PROCEDURES

### Reagents.

Recombinant Pim-1-GST and the Pim-1 peptide substrate (RSRHSSYPAGT, corresponding to amino acids 107-117 of Bad) were purchased from Millipore (Billerica, Mass.). Recombinant 4E-BP1 was purchased from Calbiochem (San Diego, Calif.), p27^{Kip1} was obtained from Novus Biologicals (Littleton, Colo.) and rapamycin was supplied by LC Laboratories (Woburn, Mass.). The following antibodies were purchased from Cell Signaling Technology (Danvers, Mass.): anti-phosphoBad (Ser112, CS-5284), anti-Bad (CS-9292), anti-p27^{Kip1} (2552), anti-β-tubulin (2146), anti-phospho4E-BP1 (Thr37/46, CS-9459), anti-4E-BP1 (9452) and from Santa Cruz Biotechnology (Santa Cruz, Calif.): anti-Pim-1 (SC-13513), anti-actin (SC-8432), anti-CDK2 (SC-163), anti-lamin B1 (SC-56144).

Treatment of Cell Lines with Pim Inhibitors.

DU145 and CWR22Rv1 (22Rv1) human prostate cancer cells overexpressing Pim-1 cDNAs were produced through retroviral transduction as described by Zemskova M. et al., in “The PIM1 Kinase Is a Critical Component of a Survival Pathway Activated by Docetaxel and Promotes Survival of Docetaxel-treated Prostate Cancer Cells.” J Biol Chem 2008; 283:20635-44. Briefly, the coding region of the human Pim-1 gene was cloned into the pLNCX retroviral vector (Clontech). To produce infectious virus, the GP-293 packaging cell line was co-transfected with retroviral plasmids (pLNCX or pLNCX/Pim-1) along with pVSV-G. After 48 hours of incubation, the virus particles were concentrated by centrifugation from the medium. Prostate cells were plated at 1×10⁵ cells/60-mm plate 16-18 h before infection cells were infected with 5×10⁴ viral particles/plate in the presence of 8 μg/ml polybrene. After 6 hours of incubation, stable pools of G418 resistant cells were selected for 10 days and the expression of the Pim-1 protein was verified by Western blot analysis. Human prostate cancer cell lines, PC3, DU145, DU145-vector, DU145-Pim, 22Rv1-vector, 22Rv1-Pim, and LNCaP, and human leukemia cell lines, MV7;11, K562, and U937, were maintained in RPMI 1640 with 10% fetal calf serum (FCS) and 1% penicillin-streptomycin at 37° C. in 5% CO₂. The IL-3-dependent murine cell line FDCP1-Pim described previously (15), was grown in RPMI 1640 with 10% FCS, 1% penicillin-streptomycin, and IL-3 (2 ng/mL) at 37° C. in 5% CO₂.

The following in vitro procedure can be used to evaluate compounds for inhibition of Pim-1 protein kinase. This procedure is referred to herein as “Procedure 1.”

Pim protein kinase assays were conducted using multiple methods to ensure that the effects of the compounds were not due to any experimental artifacts. The primary screen and evaluation of the compounds shown in Table A was conducted using an ATP-depletion assay. Recombinant human Pim-1 (available from Upstate: #14-573) was incubated with S6 kinase/Rsk-2 peptide 2 (KKRNRTLTK) (available from Upstate: #12-243) as the substrate in the presence 100 μM of the disclosed compound, 1 μM ATP and 10 mM MgCl₂ for 1 hour. The Kinase-Glo luciferase kit (Promega) was used to measure residual ATP levels after the kinase reaction. For experiments that required higher ATP concentrations, Pim-1 kinase activity was monitored spectrophotometrically using a coupled assay in which ADP production is coupled to NADH oxidation catalyzed by pyruvate kinase and lactate dehydrogenase. Assays were carried out in 20 mM MOPS pH 7 containing 100 mM NaCl, 10 mM MgCl₂, 2.5 mM phosphoenolpyruvate, 0.2 mM NADH, 30 μg/mL pyruvate kinase, 10 μg/mL lactate dehydrogenase, 2 mM dithiothreitol, 25 nM Pim-1, 100 μM S61 peptide (RRLSSLRA, American Peptide Company) and varying concentrations of ATP. Activity was measured by monitoring NADH oxidation as the decrease at 340 nm in a VersaMax microplate reader (Molecular Devices) at 25° C. Reactions were initiated by the addition of ATP (typically 100 μM). Inhibitors (final 1% DMSO) were added just prior to the addition of ATP. IC₅₀ values were determined using nonlinear regression with the program GraphPad Prism. In some experiments, Pim-1 kinase activity was determined using His-tagged 4E-BP1 as the substrate. The active Pim-1 protein (Upstate) was re-suspended in kinase reaction buffer (10 mM MOPS, pH7.4, 100 μM ATP, 15 mM MgCl₂, 1 mM Na₃VO₄, 1 mM NaF, 1 mM DTT, and protease inhibitor cocktail). In each reaction (30 μl), 3 μg of His-4E-BP1 protein was used as substrate, and 10 μCi of [γ-³²P] ATP were then added. Incubation was carried out at 30° C. for 30 min with agitation. The samples were then subjected to SDS-PAGE and ³²P labeled 4E-BP1 was visualized by autoradiography. Finally, Pim-1 activity in intact cells was measured in some experiments. HEK-293T cells were transfected with Flag-Pim-1 for 24 hours, and then were trypsined and divided into smaller dishes for overnight. Cells were washed once and incubated with phosphate-free media containing 10% phosphate-free FBS (Invitrogen, Carlsbad, Calif.) for 1 h. Cells were then incubated in medium containing 50 μCi/ml [³²P]orthophosphate for 4 hours, in which the test compounds were added for the final 1 hour. To immunoprecipitate Pim-1, anti-Flag M2 Agarose was added to the cell lysate and incubated for 3 hours. A portion (10%) of the immunoprecipitates was used for western blotting with anti-Flag antibodies (input). The other 90% of each sample was subjected to SDS-PAGE, and ³²P-labeled Pim-1 was visualized by autoradiography.

Tables 1 and 2 provide non-limiting examples of compounds and their IC₅₀ values for Pim-1 (Table 1) and Pim-2 (Table 2).

TABLE 1

| No. | Compound | IC₅₀ (μM) |
|---|---|---|
| D1 | 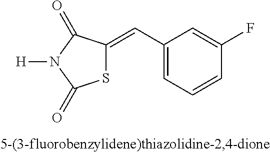 | 0.13 ± 0.06 |
| D2 | 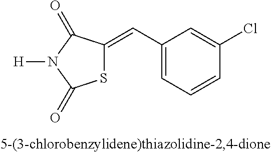 | 0.60 ± 0.51 |
| D3 | 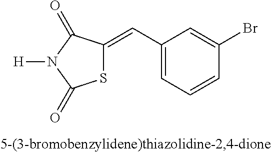 | 45 ± 11 |
| D4 | 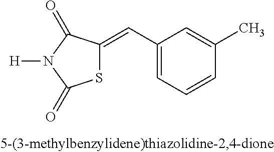 | 0.16 ± 0.04 |
| D5 | 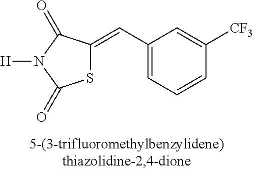 | 0.024 ± 0.006 |
| D6 | 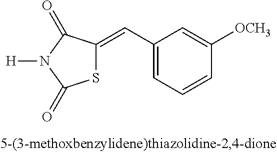 | 0.65 ± 0.05 |
| D7 | 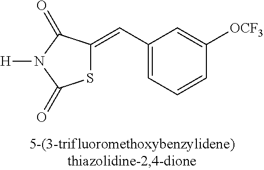 | 0.067 ± 0.061 |
| D8 | 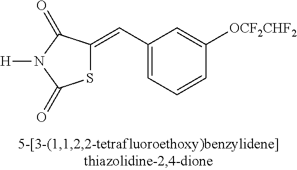 | 0.073 ± 0.053 |
| D9 | 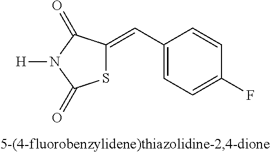 | 0.013 ± 0.01 |
| D10 | 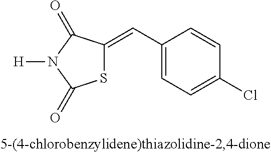 | 0.04 ± 0.03 |
| D11 | 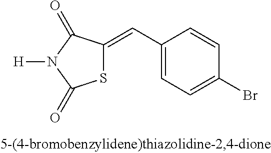 | 28 ± 23 |
| D12 | 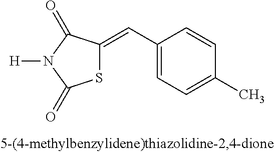 | 0.06 ± 0.02 |
| D13 | 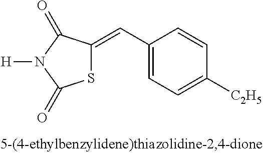 | 0.6 ± 0.1 |
| D14 | 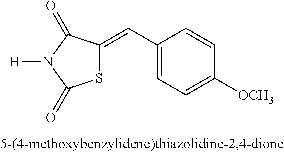 | 5.1 ± 5.0 |
| D15 | 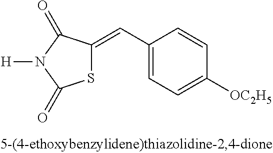 | 0.17 ± 0.04 |
| D16 | 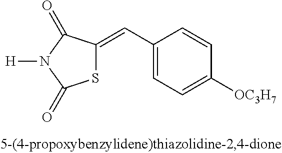 | 0.15 ± 0.11 |
| D17 | 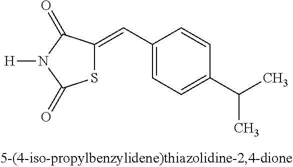 | 0.04 ± 0.03 |
| D18 | 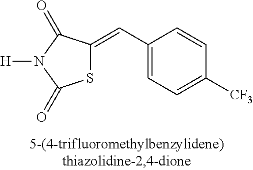 | 0.33 ± 0.13 |
| D19 | 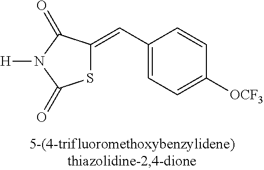 | 0.3 ± 0.2 |
| D20 | 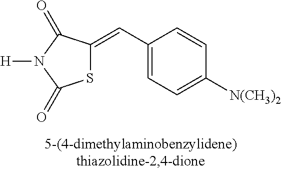 | 6.0 ± 1.7 |
| D21 | 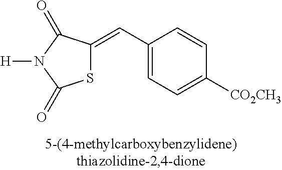 | 10.0 |
| D22 | 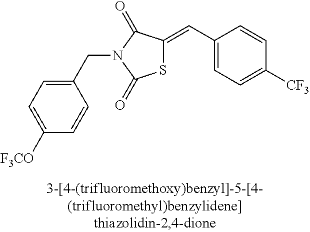 | 7.5 ± 2.5 |
| D23 | 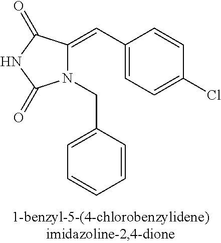 | 69 ± 6.5 |
| D24 | 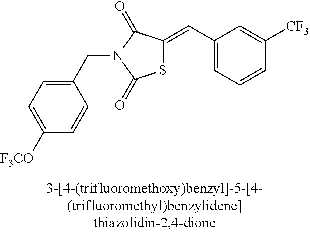 | 28 ± 22 |
| D25 | 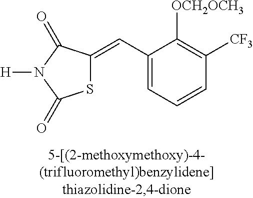 | 0.46 ± 0.32 |
| D26 | 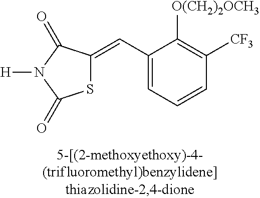 | 0.28 ± 0.13 |
| D27 | 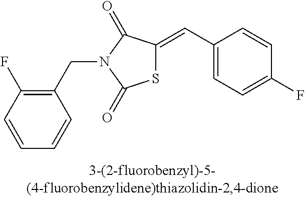 | 45 ± 15 |
| D28 | 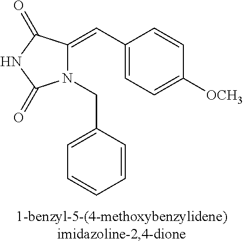 | 38 ± 23 |
| D29 | 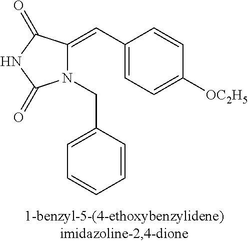 | 73 ± 7.5 |
| D30 | 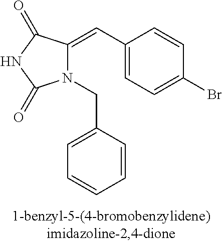 | 58 ± 7.5 |
| D31 | 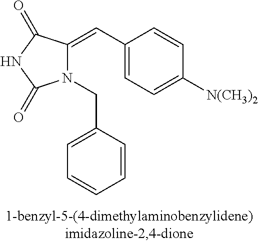 | 78 ± 7.5 |
| D32 | 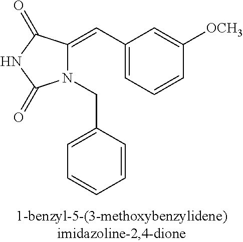 | 78 ± 2.5 |

TABLE 2

| No. | Compound | IC₅₀ (μM) |
|---|---|---|
| D1 | 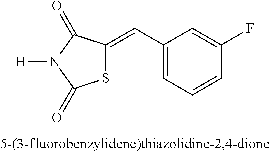 | 0.4 ± 0.2 |
| D2 | 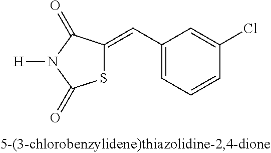 | 0.4 ± 0.15 |
| D3 | 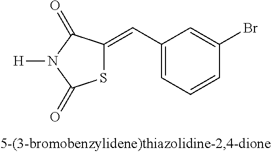 | 0.09 ± 0.04 |
| D4 | 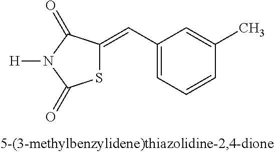 | 0.1 ± 0.1 |
| D5 | 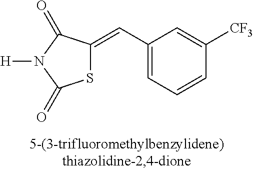 | 0.1 ± 0.3 |
| D6 | 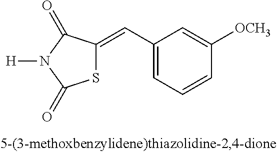 | 0.04 ± 0 |
| D7 | 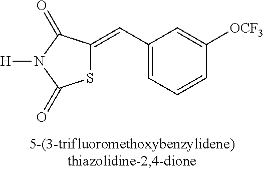 | 0.9 ± 0.4 |
| D8 | 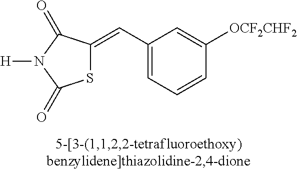 | 2.2 ± 1.1 |
| D9 | 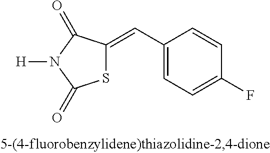 | 2.3 ± 0.1 |
| D10 | 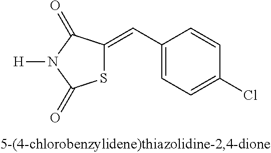 | 0.2 ± 0.1 |
| D11 | 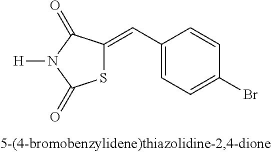 | 0.09 ± 0.04 |
| D12 | 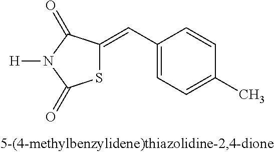 | 4.4 ± 0.2 |
| D13 | 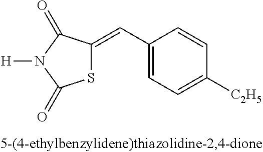 | 0.3 ± 0.1 |
| D14 | 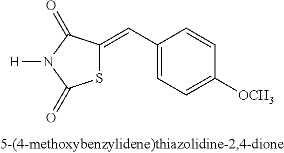 | 0.3 ± 0.1 |
| D15 | 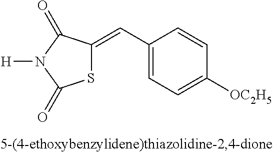 | 43 ± 18 |
| D16 | 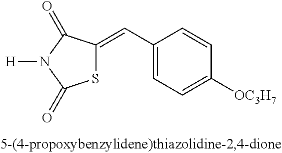 | 0.02 ± 0.01 |
| D17 | 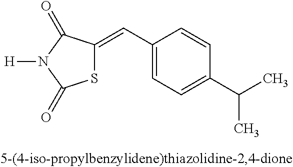 | >100 |
| D18 | 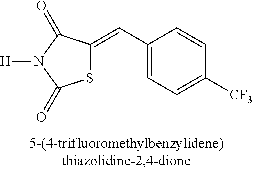 | 0.08 ± 0.02 |
| D19 | 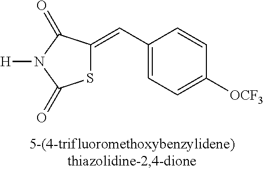 | 0.5 ± 0.2 |
| D20 | 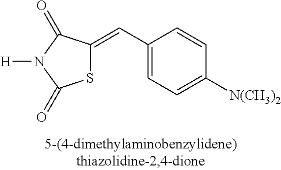 | 0.1 ± 0.1 |
| D21 | 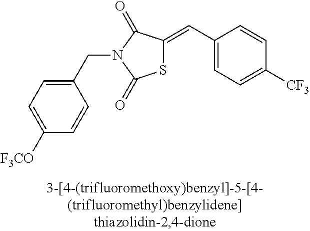 | >100 |
| D22 | 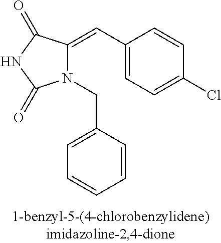 | >100 |
| D23 | 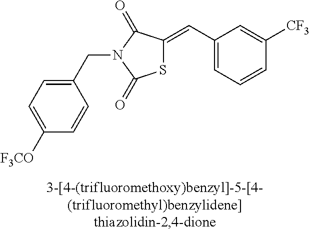 | >100 |
| D24 | 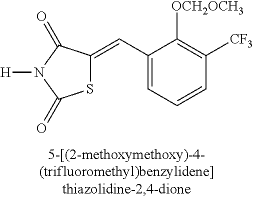 | 1.5 ± 0.1 |
| D25 | 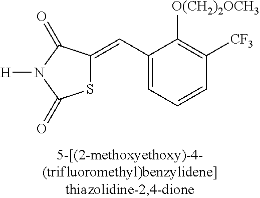 | 42 ± 12 |
| D26 | 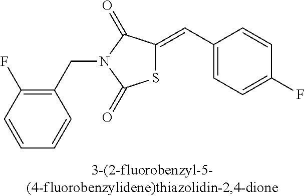 | >100 |

Cytotoxicity Assays.

Human prostate cancer PC3 cells were seeded in 96-well tissue culture dishes at approximately 10% confluency, and allowed to attach and recover for 24 hours. Varying concentrations of the test compounds are then added to each well, and the plates were incubated for an additional 48 hours. The number of surviving cells was determined by the MTS assay (Promega). The percentage of cells killed was calculated as the percentage decrease in MTS metabolism compared with control cultures. Table 3 provides IC₅₀ (μM) values for this PC3 cell assay.

TABLE 3

| No. | Compound | IC₅₀ (μM) |
|---|---|---|
| D1 | 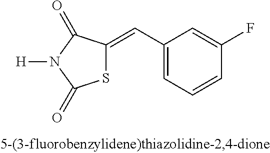 | 83 ± 9 |
| D2 | 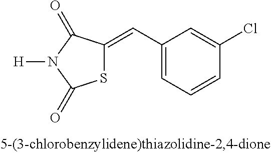 | 63 ± 6 |
| D3 | 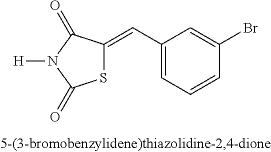 | 58 ± 11 |
| D4 | 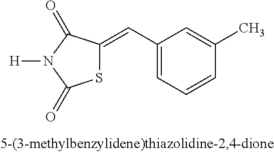 | 97 ± 0 |
| D5 | 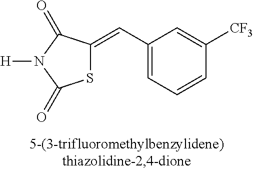 | 17 ± 6 |
| D6 | 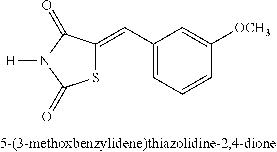 | 73 ± 13 |
| D7 | 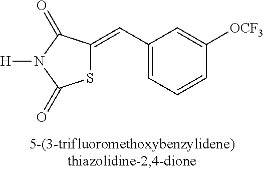 | 6.4 ± 2.4 |
| D8 | 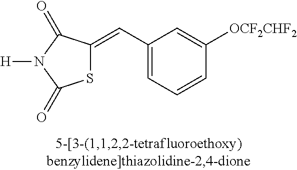 | 3.2 ± 0.5 |
| D9 | 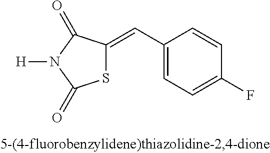 | 93 ± 5 |
| D10 | 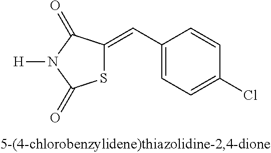 | 49 ± 8 |
| D11 | 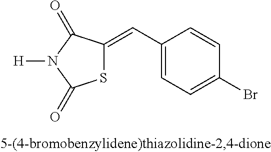 | 85 ± 12 |
| D12 | 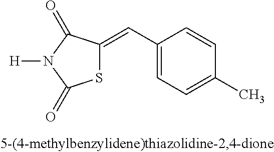 | >100 |
| D13 | 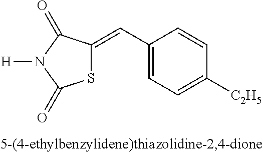 | 65 ± 18 |
| D14 | 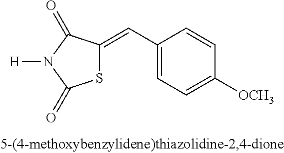 | 11 ± 1 |
| D15 | 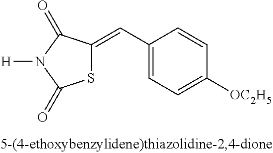 | 65 ± 17 |
| D16 | 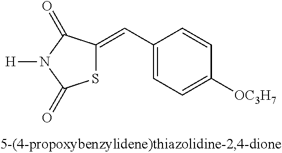 | 48 ± 8 |
| D17 | 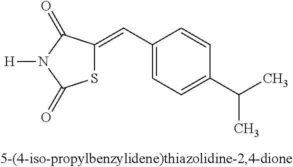 | 28 ± 13 |
| D18 | 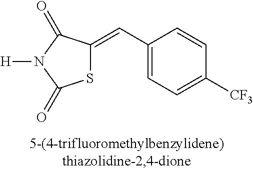 | 38 ± 31 |
| D19 | 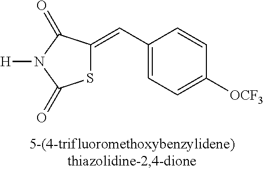 | 48 ± 27 |
| D20 | 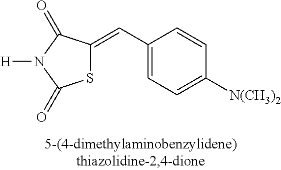 | 66 ± 13 |
| D22 | 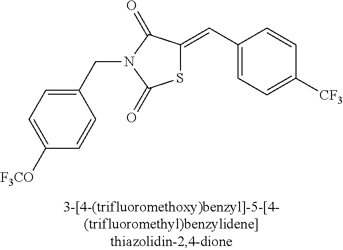 | >100 |
| D23 | 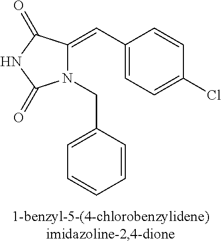 | 67 ± 28 |
| D24 | 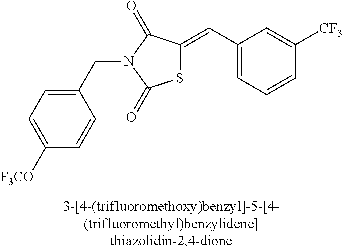 | >100 |
| D25 | 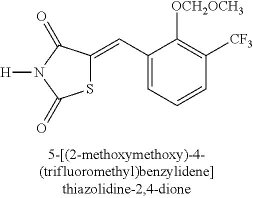 | 68 ± 18 |
| D26 | 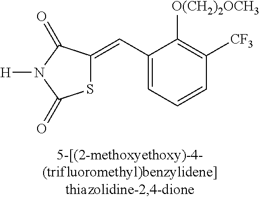 | 80 ± 12 |
| D27 | 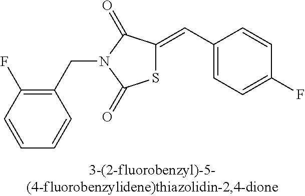 | >100 |
| D28 | 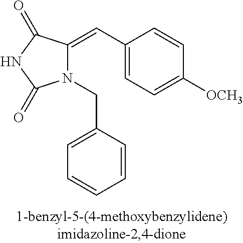 | 74 ± 20 |
| D29 | 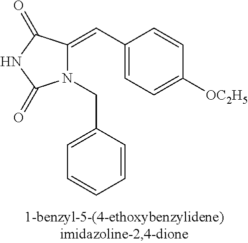 | 18 ± 9 |
| D30 | 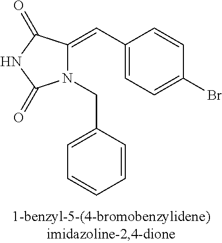 | 68 ± 22 |
| D31 | 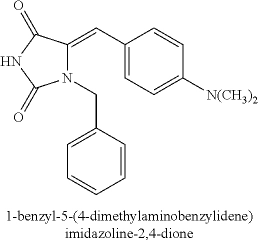 | 57 ± 23 |
| D32 | 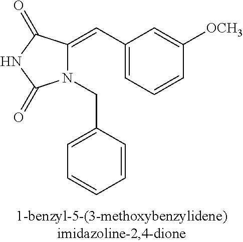 | 80 ± 15 |

Antitumor assay. A syngeneic mouse tumor model that uses a transformed murine mammary adenocarcinoma cell line (JC, ATCC Number CRL-2116) and Balb/C mice (Charles River) was performed as previously described in Lee, B. D. et al. “Development of a syngeneic in vivo tumor model and its use in evaluating a novel P-glycoprotein modulator, PGP-4008.” Oncol Res 2003, 14, (1), 49-60 included herein by reference in its entirety. Animal care and procedures were in accordance with guidelines and regulations of the IACUC of the Medical University of South Carolina. Tumor cells (1×10⁶) were implanted subcutaneously, and tumor volume was calculated using the equation: (L×W²)/2. Upon detection of tumors, mice were randomized into treatment groups. Treatment was then administered once per day, five days per week, thereafter consisting of intraperitoneal doses of 0 or 50 mg of 5-(4-iso-propylbenzylidene)thiazolidine-2,4-dione/kg or vehicle (50% DMSO:50% phosphate-buffered saline). Whole body weights and tumor volume measurements were performed three times per week. Tables 4A and 4B show the various effects of various doses of 5-(4-iso-propyl-benzylidene)thiazolidine-2,4-dione administered by intraperitoneal injection daily for 7 days wherein blood samples were collected after an additional 7 days of observation. The ranges of values for cell counts and blood chemistry are given.

TABLE 4A

| Parameter | Units | Control | 3 mg/kg |
|---|---|---|---|
| White blood cells | 10⁹/L | 3.55-9.83 | 3.73-7.99 |
| Lymphocytes | 10⁹/L | 3.44-8.88 | 3.61-6.78 |
| Monocytes | 10⁹/L | 0.04-0.36 | 0.11-0.34 |
| Granulocytes | 10⁹/L | 0.06-0.36 | 0.01-0.91 |
| Red blood cells | 10¹²/L | 10.37-11.76 | 10.37-11.33 |
| Hemoglobin | g/dL | 13.2-14 | 12.8-14 |
| Albumin | g/dL | 3.2-3.4 | 3.3-3.4 |
| Alkaline phosphatase | U/L | 87-134 | 85-127 |
| Alanine aminotransferase | U/L | 47-87 | 40-134 |
| Amylase | U/L | 926-981 | 878-1206 |
| Blood urea nitrogen | mg/dL | 15-19 | 17-19 |
| Phosphate | mg/dL | 5.7-9.5 | 5.1-5.6 |
| Creatinine | mMg/dL | 0.2-0.3 | 0.2-0.4 |
| Na⁺ | mmol/L | 150-155 | 150-155 |
| K⁺ | mmol/L | 7.7-7.9 | 7.3-7.5 |
| Glucose | mg/dL | 70-94 | 80-90 |

TABLE 4B

| Parameter | Units | 10 mg/kg | 50 mg/kg |
|---|---|---|---|
| White blood cells | 10⁹/L | 4.95-7.03 | 3.65-7.1 |
| Lymphocytes | 10⁹/L | 3.94-5.84 | 3.5-5.62 |
| Monocytes | 10⁹/L | 0.08-0.28 | 0.04-0.35 |
| Granulocytes | 10⁹/L | 0.61-0.93 | 0.08-1.13 |
| Red blood cells | 10¹²/L | 9.22-10.93 | 8.99-10.98 |
| Hemoglobin | g/dL | 11.7-14.1 | 12.2-13.5 |
| Albumin | g/dL | 2.9-3.4 | 2.6-3.1 |
| Alkaline phosphatase | U/L | 85-113 | 80-86 |
| Alanine aminotransferase | U/L | 44-46 | 37-52 |
| Amylase | U/L | 816-1054 | 760-967 |
| Blood urea nitrogen | mg/dL | 11-16 | 14-22 |
| Phosphate | mg/dL | 5.7-6.2 | 5.5-8.1 |
| Creatinine | mMg/dL | 0.2-0.3 | 0.2-0.3 |
| Na⁺ | mmol/L | 144-151 | 145-153 |
| K⁺ | mmol/L | 7.2-8.2 | 6.3-6.7 |
| Glucose | mg/dL | 87-129 | 136-163 |

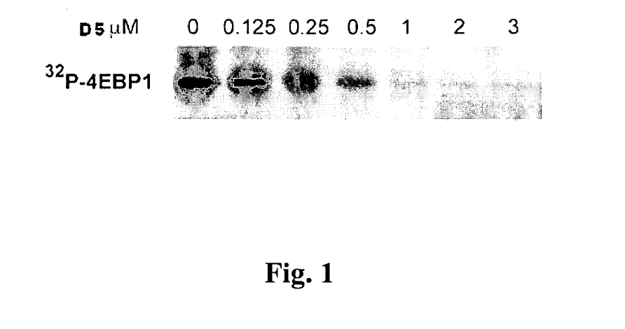

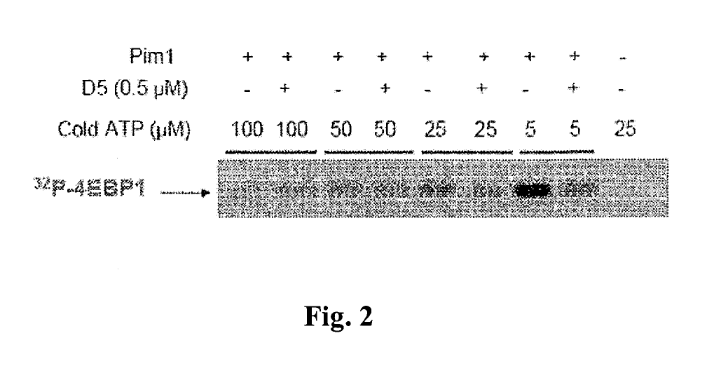

The disclosed compounds were also tested for competition with ATP, for example, the effects of 5-(3-trifluoromethylbenzylidene)thiazolidine-2,4-dione at different ATP concentrations was determined. As indicated in FIG. 1 and FIG. 2, 5-(3-trifluoromethyl-benzylidene)thiazolidine-2,4-dione acts as a competitive inhibitor with respect to ATP, with a calculated Kᵢ of 0.6 μM. The disclosed compounds can further be tested for their selectivity against other serine/threonine- or tyrosine-kinases. Table 5 provides selectivity data for 5-(3-trifluoromethylbenzyl-idene)thiazolidine-2,4-dione. As indicated in Table 5, 5 μM of 5-(3-trifluoromethylbenzyl-idene)thiazolidine-2,4-dione inhibited Pim-1 and Pim-2, but did not significantly inhibit the other 47 serine/threonine- or tyrosine-kinases tested. Similar results were obtained for 5-(4-iso-propylbenzyl-idene)thiazolidine-2,4-dione wherein this compound is highly selective for Pim kinases, although the kinase DYRK1α was inhibited to a similar extent as Pim-1 and Pim-2.

TABLE 5

| Kinase | Comp¹ | Comp² |
|---|---|---|
| ABL | −4 | 6 |
| AKT1 | −9 | 6 |
| AKT2 | 3 | −1 |
| AKT3 | 7 | 3 |
| AMPK | 2 | 7 |
| AurA | 6 | 4 |
| BMX | 8 | 10 |
| BTK | 10 | 7 |
| CAMK2 | −6 | −1 |
| CAMK4 | 10 | −8 |
| CDK2 | 0 | −2 |
| CHK1 | −10 | −20 |
| CHK2 | 1 | 4 |
| CK1δ | 3 | 7 |
| c-Raf | 9 | −1 |
| c-TAK1 | 5 | 0 |
| DYRK1α | 20 | 68 |
| EGFR | −1 | −3 |
| Erk1 | 2 | 1 |
| Erk2 | 4 | −1 |
| FGFR1 | 3 | 1 |
| FLT1 | 18 | NT |
| FLT3 | −2 | 1 |
| FLT3(D835Y) | 12 | 9 |
| FYN | 3 | 0 |
| GSK3β | −1 | 3 |
| HGK | −14 | 9 |
| IGF1R | 1 | −1 |
| Ikkβ | 12 | 10 |
| INSR | 5 | 4 |
| IRAK4 | 16 | 5 |
| JAK2 | 15 | 18 |
| JNK2 | 4 | 14 |
| KDR | 14 | −4 |
| LCK | 2 | 3 |
| LYN | 3 | 8 |
| MAPKAPK2 | 0 | 5 |
| MARK1 | 5 | −6 |
| MET | 3 | −6 |
| MSK1 | −3 | −1 |
| MST2 | 2 | 1 |
| p38α | −2 | 0 |
| p70S6K | 10 | 8 |
| PAK2 | 3 | −1 |
| PDGFRα | 7 | 6 |
| PDK1 | 17 | 1 |
| PIM1 | 73 | 36 |
| PIM2 | 68 | 62 |
| PKA | 2 | 3 |
| PKCβ2 | −2 | −1 |
| PKCζ | −2 | 3 |
| PKD2 | −6 | −3 |
| PKGα | 1 | 0 |
| PLK1 | 3 | 9 |
| PRAK | −3 | 4 |
| ROCK2 | 5 | 5 |
| RSK1 | 3 | 5 |
| SGK1 | 0 | 5 |
| SRC | 4 | 4 |
| SYK | 5 | −3 |

¹Results for 5-(3-trifluoromethylbenzyl-idene)thiazolidine-2,4-dione
²Results for 5-(4-iso-propylbenzyl-idene)thiazolidine-2,4-dione

### Western Blotting.

Cells were harvested, washed with PBS and resuspended in lysis buffer (20 mM Tris-HCl pH 7.5 containing 1% SDS, 50 mM NaCl, 1 mM EDTA, 1 mM phenylmethyl-sulfonyl fluoride, 10 mM sodium fluoride, 1 mM sodium orthovanadate). Samples were then incubated on ice for 30 minutes followed by 15 min centrifugation. Supernatants were separated by SDS-PAGE and transferred to nitrocellulose membranes. Membranes were blocked in 5% nonfat milk in TBST (20 mM Tris-HCl pH 7.5 containing 150 mM NaCl, 0.1% Tween-20) for 1 hour with agitation, washed, and primary antibodies were added (1:1000 dilution in 5% bovine serum albumin in TBST) and membranes were incubated overnight at 4° C. with agitation. Membranes were washed and incubated with horseradish peroxidase conjugated secondary antibodies (1:5000 dilution in 5% nonfat milk in TBST) for 2 hours at room temperature with agitation. Proteins were detected using the ECL Western Blotting Detection Reagent (GE Healthcare, Piscataway, N.J.).

p27^{Kip1} Location and Cdk2 Kinase Activity Assays.

To examine p27^{Kip1} location, K562, U937 or MV7;11 cells (1×10⁵/mL) were incubated for 72 hours in complete media with DMSO or a disclosed Pim-1 and/or Pim-2 inhibitor. Cells were harvested, washed in PBS and cytoplasmic and nuclear fractions were prepared using the NE-PER Nuclear and Cytoplasmic Extraction kit (Pierce Biotechnology, Rockford, Ill.) according to the manufacturer's instructions, followed by SDS-PAGE and western blotting with anti-p27^{Kip1} antibody, as described above. To measure Cdk2 activity, this protein was immunoprecipitated from K562, U937, or MV7;11 cells treated for 72 hours with Pim inhibitors, lysed in buffer (50 mM Tris-HCl, pH 8.0 containing 5 mM EDTA, 150 mM NaCl, 1% NP-40 and 1 mM phenylmethylsulfonyl fluoride) followed by the addition of Cdk2 antibody (2 μg). Samples were then rotated overnight at 4° C., and Cdk2 was immunoprecipitated by the addition of protein G beads (Pierce Biotechnology) with rotation at room temperature for 1 h. Beads were washed three times with PBS and resuspended in assay buffer (10 mM MOPS, pH 7.2 containing 1 mM EDTA, 15 mM MgCl₂, 10 mM sodium fluoride, 1 mM sodium orthovanadate) containing histone H1 (3 μg, Millipore) as a Cdk2 substrate, ATP (100 μM), and [γ-³²P]-ATP (10 μCi). Reactions were allowed to proceed for 15 minutes at 37° C., and then analyzed by SDS-PAGE. ³²P-Phosphorylated histone H1 was visualized by autoradiography, and Cdk2 protein levels detected by Western blotting, as described above.

To examine p27^{Kip1} location by fluorescence microscopy DU145-vector and DU145-Pim cells were transfected with plasmids pEYFP-C1, pEYFP-p27^{Kip1}, pEYFP-p27^{Kip1}(T157A), or pEYFP-p27^{Kip1}(T198A) (1 μg DNA per well in a 6-well dish) using lipofectamine 2000 (Invitrogen, Carlsbad, Calif.). Forty-eight hours after transfection, cells were treated with a disclosed Pim-1 and/or Pim-2 inhibitor (5 μM) in DMEM containing 1% FCS for 24 hours. The expression of EYFP-p27^{Kip1} in live cells was visualized on a Leica TCS SP2 laser scanning confocal microscope (Leica Microsystems, Wetzler, Germany).

The recombinant HA-tagged p27, wild type and mutants, were generated by PCR, sequenced, and cloned into pcDNA3.1 between Hind III and EcoRV restriction sites. The plasmids were transfected into K562 cells with lipofectamine, harvested after 48 hours of incubation, and subjected to cytosolic and nuclear fractionation.

### METHODS

As stated herein above, the TOR protein kinase is found in two complexes, TORC1 and TORC2. The TORC1 complex controls protein synthesis by phosphorylating the 4E-BP1 protein at threonine 37 and 46. This phosphorylation releases 4E-BP1 from eIF4E allowing cap-dependent transcription to take place. TORC1 also phosphorylates p70S6 protein kinase, which on activation, phosphorylates the S6 protein, and this is critical for translation. In contrast, the TORC2 complex phosphorylates S473 of the Akt protein kinase allowing a second phosphorylation by the PDK1 kinase at T308 to occur and for Akt to be activated.

It has now been shown that when in the human PC3 prostate cancer cells the Pim-1 and Pim-2 proteins are over-expressed, therefore, 4E-BP1phosphorylation is enhanced. In addition, dominant-negative Pim can inhibit growth factor-induced 4E-BP1 phosphorylation and decrease PC3 tumor formation. It has now been discovered that the disclosed compounds can inhibit Pim-1 protein kinase activity. Also, the disclosed compounds can enhance the activity of rapamycin leading to more complete inhibition of 4E-BP1 phosphorylation and TOR activity. As a consequence, there is decreased p70S6 kinase activity and increased phosphorylation of Akt on S473.

It has further been shown (Zippo, A. et al., (2007) “Pim1-dependent phosphorylation of Histone H3 at Serine 10 is required for MYC-dependent transcriptional activation and oncogenic transformation.” Nature Cell Biology, 9:932) that inhibition of Pim-1 acts to block the formation of the Pim-1 complex with Myc/Max. The c-myc gene, which induces cell proliferation, has been found to be involved in cancer, thus inhibiting phosphorylation of serine 10 of histone H3; this further provides a method for treating cancer.

The disclosed compounds block the ability of Pim to phosphorylate peptides and proteins in vitro, and when added to DU145 prostate cancer cells overexpressing Pim, inhibit the ability of this enzyme to phosphorylate a known substrate, the BH₃ protein BAD. When added to prostate cancer cell lines, including PC-3, DU145 and 22Rv1, and human leukemic cells, MV7;11, K562 and U937 cells, these compounds induce G1/S cell cycle arrest and block the anti-apoptotic effect of the Pim protein kinase. The cell cycle arrest induced by these compounds is associated with an inhibition of cyclin-dependent kinase-2, Cdk2, activity and translocation of the Pim-1 substrate p27^{Kip1}, a Cdk2 inhibitory protein, to the nucleus. In addition, when added to leukemic cells the disclosed compounds synergize with the mTOR inhibitor rapamycin to decrease the phosphorylation level of the translational repressor 4E-BP1 at sites phosphorylated by mTOR. Combinations of rapamycin and the disclosed compounds block the growth of leukemic cells.

Pim has been shown to regulate nuclear factor-kappa B (NF-κB) activity and therefore regulate additional downstream proteins involved in apoptosis, i.e. Bax (Hammerman P S, et al. Lymphocyte transformation by Pim-2 is dependent on nuclear factor-kappaB activation. Cancer Res 2004; 64:8341-8). Pim protein kinase has been shown to phosphorylate substrates involved in cell cycle progression including Cdc25A, p21, p27^{Kip1}, NuMA, C-TAK1, and Cdc25C, whose phosphorylation results in G1/S and/or G2/M progression (Amaravadi R. et al., The survival kinases Akt and Pim as potential pharmacological targets. J Clin Invest 2005; 115:2618-24; Zhang Y. et al., Pim-1 kinase-dependent phosphorylation of p21Cip1/WAF1 regulates its stability and cellular localization in H1299 cells. Mol Cancer Res 2007; 5:909-22; Bachmann M. et al., The serine/threonine kinase Pim-1. Int J Biochem Cell Biol 2005; 37:726-30; and Morishita D. et al., Pim kinases promote cell cycle progression by phosphorylating and down-regulating p27Kip1 at the transcriptional and posttranscriptional levels. Cancer Res 2008; 68:5076-85). Also, Pim-2 has been shown to regulate the phosphorylation of 4E-BP1 causing it to dissociate from eIF-4E, suggesting a potential indirect control mechanism of cell growth. In tissue culture, serum starved PC3 cells showed cell cycle arrest in G1, while PC3-Pim cells showed much lower extent of arrest (Chen W. W. et. al., Pim family kinases enhance tumor growth of prostate cancer cells. Mol Cancer Res 2005; 3:443-51). When these cells were grown as subcutaneous tumors in mice, PC3 prostate cancer cells overexpressing Pim-1 grew significantly faster than cells expressing vector control, again pointing to a role of Pim in enhancing cell growth rate.

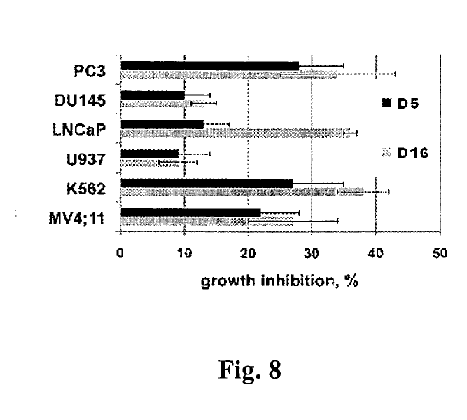

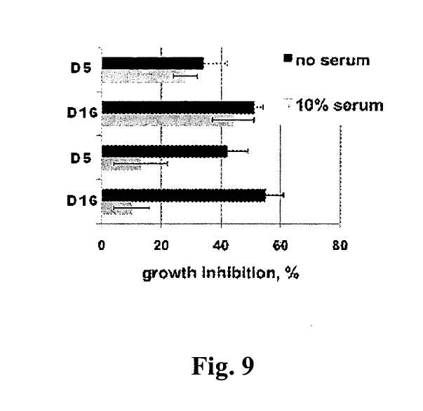

The disclosed compounds were screened using the S6 kinase/RSK-2 peptide as a substrate. The following provides non-limiting examples of cell based assays which examined the ability of the disclosed compounds to inhibit the autophosphorylation of Pim-1 protein kinase transfected in HEK 293 cells. The disclosed compounds can be tested in the following cell based assays for the percent growth inhibition of each compound using the prostate cancer cell line PC3 at a single dose of 5 μM after 24 hours as indicated in Table 3. The disclosed compounds can be tested in a coupled kinase assay using a peptide corresponding to amino acids 107-117 of the pro-apoptotic protein Bad (RSRHSSYPAGT) a known in vivo substrate of Pim kinase. For example, disclosed compounds D5 and D16 had Pim-1 IC₅₀ inhibition values of 17±7 nM for D5 and 63±11 nM for D16. In addition, compounds can be tested for competitive inhibition with respect to ATP in order to determine the extent that they bind within the ATP-binding pocket. As depicted in FIG. 1, D5 inhibited the in vitro phosphorylation by Pim-1 of the known substrate, the translational repressor 4E-BP1. The ability of D5 and D16 to inhibit the growth of various cancer cell lines was evaluated after treatment for 72 hours in culture. Prostate cancer and leukemic cell lines were chosen since Pim-1 has been shown to play an integral role in the development of prostate carcinogenesis and hematological malignancies (Cibull T. L. et al., Overexpression of Pim-1 during progression of prostatic adenocarcinoma. J Clin Pathol 2006; 59:285-8; Dhanasekaran S. M. et al., Delineation of prognostic biomarkers in prostate cancer. Nature 2001; 412:822-6; Ellwood-Yen K. et al. Myc-driven murine prostate cancer shares molecular features with human prostate tumors. Cancer Cell 2003; 4:223-38; Kim K. T. et al. Constitutive Fms-like tyrosine kinase 3 activation results in specific changes in gene expression in myeloid leukaemic cells. Br J Haematol 2007; 138:603-15; Adam M, et al., Targeting PIM kinases impairs survival of hematopoietic cells transformed by kinase inhibitor-sensitive and kinase inhibitor-resistant forms of Fms-like tyrosine kinase 3 and BCR/ABL. Cancer Res 2006; 66:3828-35; and Hammerman P. S. et al., Pim and Akt oncogenes are independent regulators of hematopoietic cell growth and survival. Blood 2005; 105:4477-83). As depicted in FIG. 8, D5 and D16 caused growth inhibition of each cell line. The sensitivity to D5 and D16 was not affected by withdrawal of serum from PC3 cells, however, as depicted in FIG. 9, DU145 cells became considerably more sensitive under serum-free conditions.

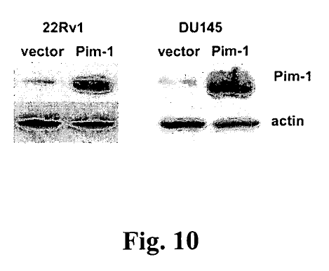

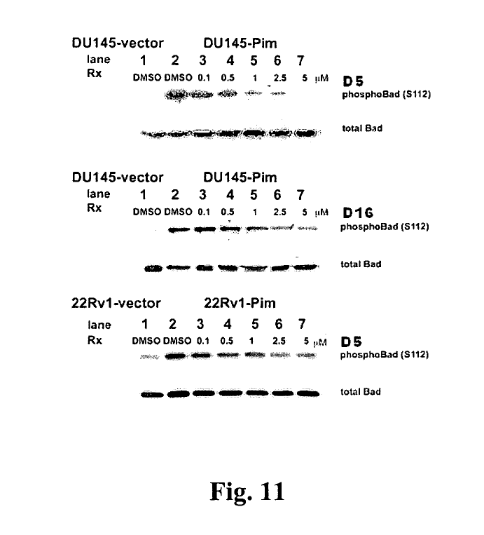

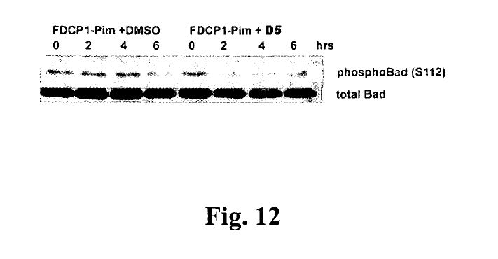

The phosphorylation level of the Pim target Bad can also be determined. For example, the phosphorylation level of the Pim target Bad by D5 and D16 was determined by Western blotting using prostate cancer and hematopoietic cells stably transfected with Pim-1. As depicted in FIG. 10, the 22Rv1-vector cells show more endogenous Pim-1 protein compared to DU145-vector cells and, as depicted in FIG. 11 more endogenous phosphorylated Bad protein (phosphoBad). The level of phosphoBad decreased in a dose-dependent manner in both 22Rv1-Pim and DU145-Pim cells treated with D5 or D16 for 1 hour under serum-free conditions, while the level of total Bad protein remained constant. The FDCP1-Pim cell line has been shown to survive longer with fewer apoptotic cells compared to the FDCP1-vector cell line (Lilly M. et al., Enforced expression of the Mr 33,000 Pim-1 kinase enhances factor-independent survival and inhibits apoptosis in murine myeloid cells. Cancer Res 1997; 57:5348-55). As such, the level of phosphoBad can be examined over a time course in the hematopoietic cell line FDCP1 stably transfected with Pim-1 in the absence (DMSO) or presence of one of the disclosed compounds, for example, D5 (5 μM) in serum and IL-3-free conditions. As depicted in FIG. 12, D5 shows a reduction in phosphoBad levels in Pim inhibitor-treated FDCP1-Pim cells by 2 hours when compared to DMSO-treated cells.

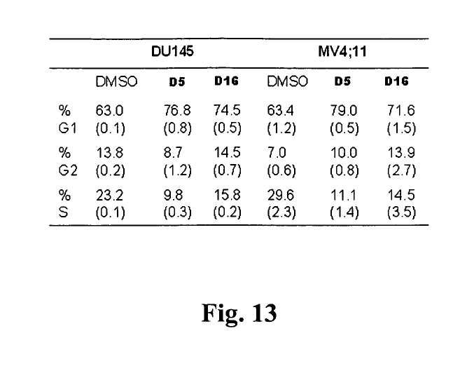

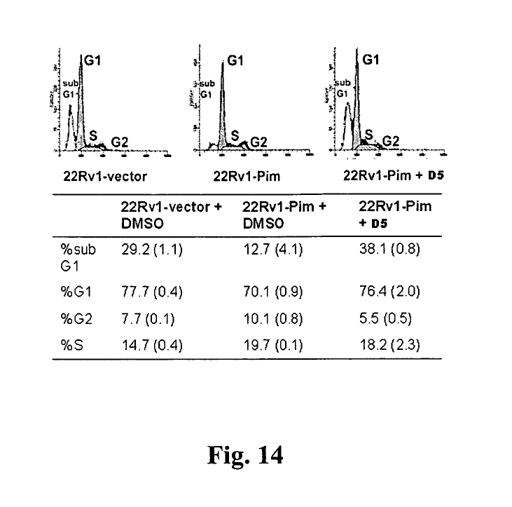

The disclosed compounds can also be evaluated for cell cycle arrest and reverse the anti-apoptotic activity of Pim-1. Many Pim-1 substrates play a role in cell cycle progression including Cdc25A, p21, p27^{Kip1}, NuMA, C-TAK1 and Cdc25C which when phosphorylated result in G1/S and/or G2/M progression. Therefore, the ability of the disclosed compounds to affect the cell cycle distribution of both prostate cancer and hematopoietic cells can be determined. D5 and D16 were evaluated for their ability affect the cell cycle distribution of both prostate cancer and hematopoietic cells. DU145 growing in 2% serum and MV7;11 cells plated in 10% serum were treated with D5 or D16 at 5 μM for 72 hours followed by FACS analysis. As depicted in FIG. 13, both of these compounds caused a significant G1 cell cycle arrest compared to the DMSO control. No significant sub-G1 population (apoptotic cells) was observed in either cell line. In addition, the apoptotic effect of D5 was shown using the 22Rv1-vector and 22Rv1-Pim cell lines. In FIG. 14, cells were treated with DMSO or D5 (5 μM) for 72 hours under serum-free conditions. Serum starvation of 22Rv1-vector (+DMSO) resulted in apoptosis (sub G1 29.2%); however, expression of Pim-1 decreased the percent of apoptotic cells (12.7%) consistent with its pro-survival role as previously determined in myeloid cells (Lilly M. et al.). Treatment of 22Rv1-Pim cells with D5 reversed the anti-apoptotic effect of Pim-1 as the sub G1 population increased to 38.1% (compared to 12.7% for DMSO treated cells). Additionally, the cell cycle analysis demonstrates that overexpression of Pim-1 decreases the percentage of cells in G1 and increases the number in S and G2. This Pim-1 effect is reversed by treatment with D5 or D16 demonstrating their ability to induce a G1 block.

Control of p27^{Kip1} in the Nucleus

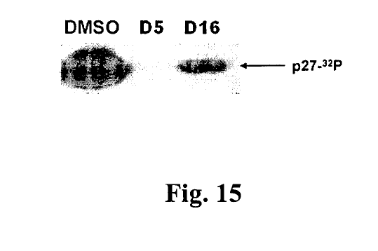

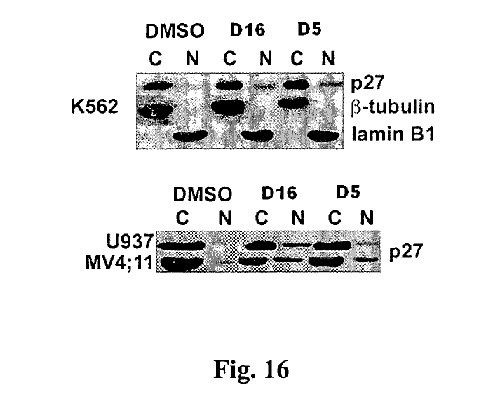

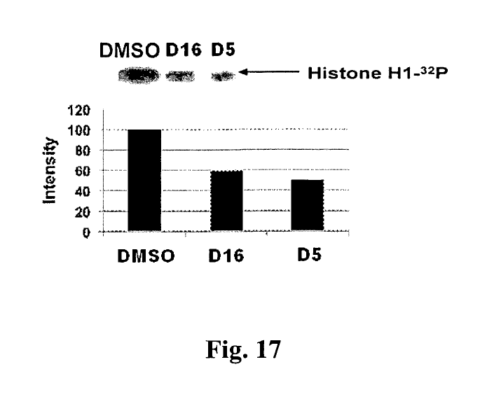

The disclosed compounds can also be evaluated for their ability to increase the amount of p27^{Kip1} in the Nucleus thereby resulting in its nuclear export and degradation. FIGS. F and G depicted the ability of D5 and D16 to induce cell cycle arrest. FIG. 15 depicts the ability of Pim-1 to phosphorylate p27^{Kip1} and the ability of D5 and D16 (5 μM) to reduce phosphorylation of this substrate was demonstrated in vitro. The leukemic cell lines K562, U937, and MV7;11 were treated with D5 or D16 for 72 hours in media containing 10% FCS, followed by detection of p27^{Kip1} levels in cytoplasmic and nuclear fractions (FIG. 16). Both of these compounds caused an increase in the amount of p27^{Kip1} in nuclear fractions in all three cell lines. This fact demonstrates that overexpression of Pim-1 in K562 cells promoted cell cycle progression by up-regulating Cdk2 activity. The affect of Pim-1 inhibition by D5 and D16 on Cdk2 activity was then determined. K562 cells were treated under the same conditions in FIG. 17, Cdk2 was immunoprecipitated and its kinase activity determined using histone H1 as the substrate. Cdk2 immuno-precipitated from D5 or D16 treated cells showed ˜50% and 60%, respectively decreased activity. Kinase selectivity profiling demonstrated that D5 and D16 do not inhibit Cdk2 activity. As such, these results are consistent with inhibition of endogenous Pim-1 by these the disclosed compounds causing increased nuclear p27^{Kip1} levels, and inhibiting Cdk2 activity.

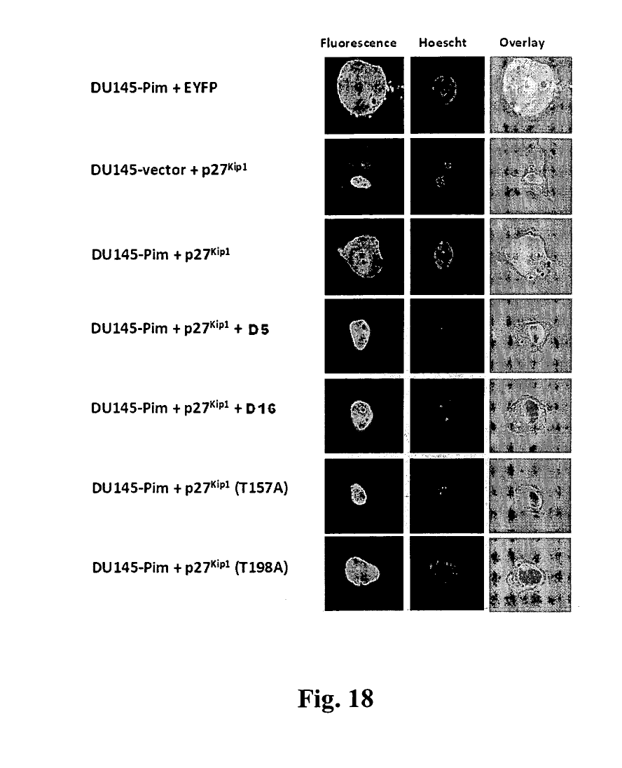

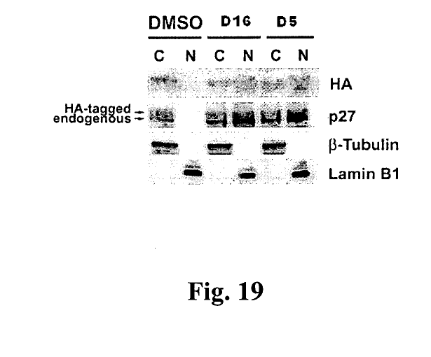

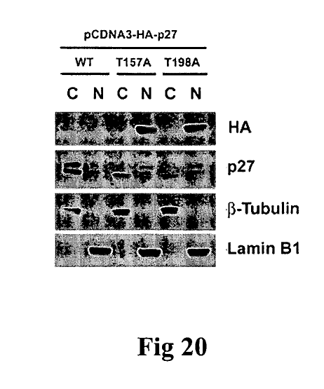

To determine the effect of Pim-1 overexpression on p27^{Kip1} localization, DU145-vector and DU145-Pim cells were transfected with a plasmid expressing p27^{Kip1} fused to enhanced yellow fluorescent protein (EYFP) and p27^{Kip1} was then visualized by fluorescence microscopy. As shown in FIG. 18, the control vector expressing EYFP alone is distributed throughout the nucleus and cytosol while the fusion with p27^{Kip1} localizes the fluorescence in the nucleus as demonstrated by overlay with Hoescht dye which stains nuclei. Overexpression of Pim-1 in the DU145 cells increased the amount of p27^{Kip1} located in the cytosol. Treatment of these cells with the compounds D5 or D16 reversed this Pim-mediated effect, as shown by the decreased cytosolic p27^{Kip1} after treatment. Mutation of T157A or T198A, two hypothesized Pim phosphorylation sites targeted this protein to the nucleus. As depicted in FIG. 19, using cell fractionation and western blotting, similar results were obtained in K562 leukemia cells transfected with HA-tagged p27^{Kip1}. Pim-1 has been shown to phosphorylate p27^{Kip1} at T157 and T198, which is postulated to promote p27^{Kip1} nuclear export (Morishita D. et al., Pim kinases promote cell cycle progression by phosphorylating and down-regulating p27Kip1 at the transcriptional and posttranscriptional levels. Cancer Res 2008; 68:5076-85). Accordingly, mutation of either T157 or T198 to alanine resulted in a mutant p27^{Kip1} that localized exclusively to the nucleus in K562 cells demonstrating similar results to the Pim-1 overexpressing DU145 cells (FIG. 18 and FIG. 20). These results are consistent with the inhibition of Cdk2 phosphorylation by D5 or D16 causing nuclear retention of p27^{Kip1}.

Disclosed Compounds and mTOR Inhibitors

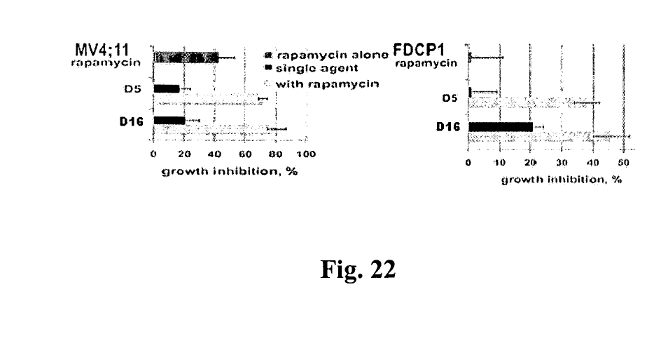

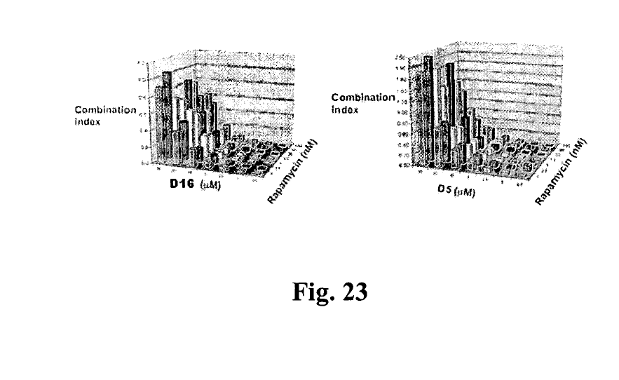

The ability of the disclosed compounds when used with mTOR inhibitors, inter alia, rapamycin to inhibit leukemic cells can be determined as follows. Upon addition of serum or growth factors, the translational repressor 4E-BP1 is inactivated by hyperphosphorylation, in part through the activity of mTOR on Thr37 and Thr46 of 4E-BP1, allowing for increased protein synthesis. Phosphorylation of these sites is sensitive to treatment with the mTOR inhibitor Rapamycin (Chen W. W. et al., Pim family kinases enhance tumor growth of prostate cancer cells. Mol Cancer Res 2005; 3:443-51). 4E-BP1 is a known in vitro target of the Pim kinases, although the mechanism by which Pim affects this protein in vivo has not been clearly defined. As depicted in FIG. 1, D5 and D16 inhibit the in vitro Pim-mediated phosphorylation of 4E-BP1. FDCP-1 cell line that is IL-3 dependent can be used to evaluate the role of combined treatment of rapamycin and the disclosed compounds. To evaluate the effects of D5 and rapamycin, these cells were starved of serum and IL-3 for 1 hour during which rapamycin (20 nM) or D5 at various concentrations, or in combination were added. At the end of this incubation, IL-3 was added to stimulate 4E-BP1 phosphorylation. The cells were centrifuged and extracts subjected to SDS-PAGE and Western blotting. Using an antibody to the Thr37/46 phosphorylation site of 4E-BP1, increasing D5 concentrations reduced the level of the most highly phosphorylated form of 4EBP1 (FIG. 22, upper arrow) and when combined with rapamycin also decreased the less phosphorylated forms of 4E-BP1 (FIG. 22, lower arrow). This combined effect is seen in the 4E-BP1 blot as an increase in the lower band. Similar regulation of 4E-BP1 phosphorylation was seen with MV7;11 cells. As depicted in FIG. 23, the combined treatment of rapamycin with D5 or D16 for 72 hours caused significant growth inhibition of MV7;11 and FDCP1 cells with D16 showing slightly more combined inhibitory effect than D5. To examine the potential synergistic growth inhibitory effect between D5 or D16 and rapamycin in MV7;11 cells, a combination index analysis was carried out (FIG. 24). These results demonstrate that at low doses of one or more of the disclosed compounds and rapamycin the combined effect of these agents is highly synergistic, while at higher concentrations of D5 and D16 this synergism is lost. As shown in Table 5, D16 inhibits DYRK1a. The effect of the combination of one or more of the disclosed compounds and rapamycin can be determined by treating MV7;11 cells with harmine and rapamycin and determining the growth inhibition compared with harmine alone.

As such, the present disclosure relates to a method for treating cancer, comprising, administering to a human an effective amount of one or more compounds that inhibit Pim-1 activity.

The present disclosure also relate to a method for treating prostate cancer, comprising, administering to a human an effective amount of one or more compounds that inhibit the formation of the Pim-1 complex with myc/max.

As discussed herein above, phosphorylation of 4E-BP1 is enhanced in PC3 prostate cancer cells due to the increased expression of Pim-1. FIG. 1 depicts the dose response for Pim-1 kinase inhibition in the presence of an inhibitor as disclosed herein using 4E-BP-1 as the substrate. His-tagged 4E-BP-1 was incubated with 0.1 μg Pim-1 protein kinase for 1 hour at 30° C. together with [γ-³²P]ATP, Mg²⁺, and cold ATP with from 0.125 to 3 μM of 5-(3-trifluoro-methylbenzylidene)thiazolidine-2,4-dione (D5). As depicted in FIG. 1, 5-(3-trifluoromethyl-benzylidene)thiazolidine-2,4-dione caused a doe-dependent reduction in Pim-1 induced 4E-BP1 phosphorylation with an IC₅₀ of approximately 0.25 μM. This test is referred to herein as “Procedure 2.”

The present disclosure relates to a method for inhibiting the phosphorylation of 4E-BP 1 in cancer cells, comprising, contacting an effective amount of one or more compounds according to the present disclosure with cancer cells in vitro, in vivo, or ex vivo.

The present disclosure further relates to a method for inhibiting the growth of prostate cancer in a human, comprising, administering to a human an effective amount of one or more compounds according to the present disclosure.

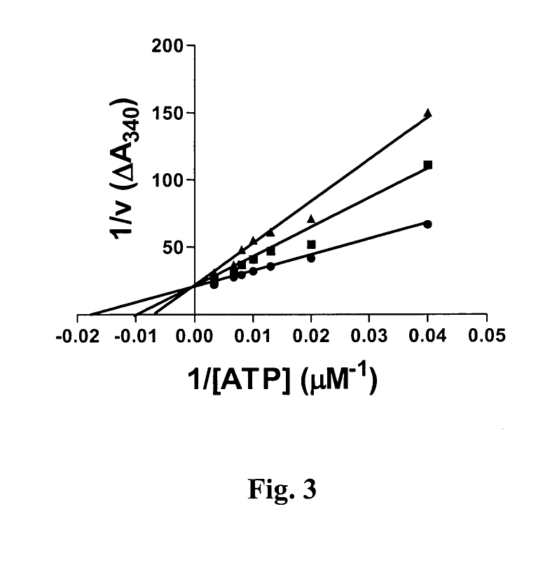

Procedure 2, described herein above, was modified to determine the mechanism of action for Pim-1 inhibition. FIG. 2 depicts the effect of varying concentrations of cold ATP in Procedure 2. Inhibition of Pim-1 activity by 0.5 μM 5-(3-trifluoromethyl-benzylidene)thiazolidine-2,4-dione (D5) was more effective at low concentrations of ATP. The inhibitory effect of D5 was lost when the total ATP concentration exceeded 100 μM, thus indicating D5 to be a competitive inhibitor with respect to ATP. FIG. 3 depicts the Lineweaver-Burke plot for the experiment depicted in FIG. 2. These data suggest D5 exhibits a Kᵢ of approximately 70 nM.

As described herein above, the TOR protein kinase controls protein synthesis by phosphorylating the 4E-BP1 protein at threonine 37 and 46. FDCP1 cells, which are IL-3-dependent myeloid progenitors that differentiate into monocytes when cultured in granulocyte macrophage-colony-stimulating factor, were incubated with 5-(3-trifluoromethylbenzylidene)-thiazolidine-2,4-dione (D5) and/or rapamycin, in order to test the activity of the disclosed Pim-1 inhibitors in the presence of rapamycin.

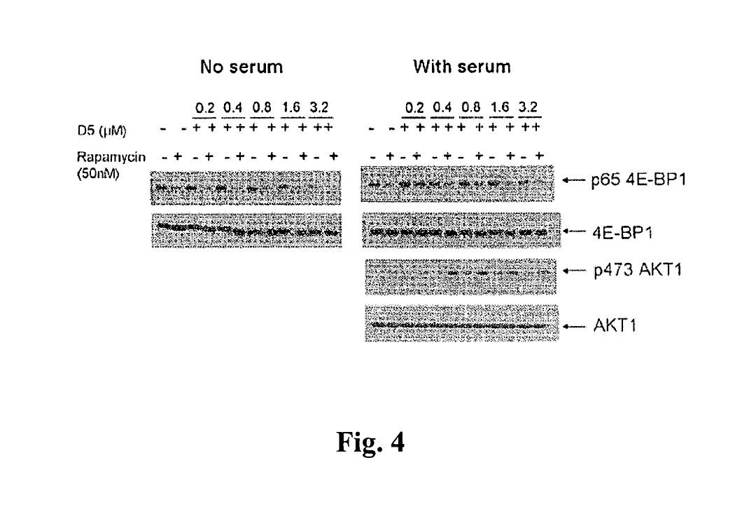

FDCP1 cells were washed free of IL-3 and serum then incubated with IL-3; IL-3 and serum; and various doses of D5 and/or rapamycin for 1 hour. FIG. 4 depicts the western blot of the FDCP1 cell lysates. As shown in FIG. 4, with or without serum, D5 enhanced the ability of rapamycin to inhibit 4E-BP1 phosphorylation. At 1.6-3.2 μM, D5 inhibited the phosphorylation of 4E-BP1 absent rapamycin. Thus D5 acts as a complement to rapamycin. In addition, D5 inhibits TOR activity and decreases p70S6K1 activity.

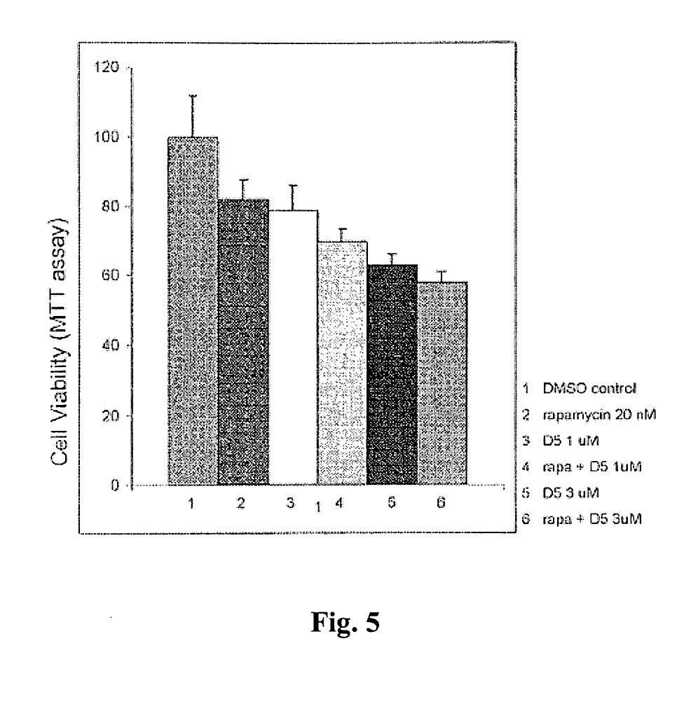

5-(3-Trifluoromethylbenzylidene)-thiazolidine-2,4-dione (D5) and PC-3 prostate cancer cell were incubated together at D5 doses of 1 and 3 μM with or without rapamycin (20 nM). FIG. 5 depicts the results of these experiments. D5 was able to enhance rapamycin's ability to inhibit PC-3 cell viability, as well as being able to inhibit cell viability by 40% after 36 hours when administered alone.

It was found that the disclosed compounds, for example, D5 and D16 inhibit the TOR protein kinase and decrease 4EBP1 phosphorylation either alone or in combination with rapamycin. D5 and D16 increase the phosphorylation of the AMPK protein kinase on threonine 172. This phosphorylation is known to activate this protein kinase and lead to the phosphorylation of TSC2 and the inhibition of TOR protein kinase (Molecular Cell 30: 214-226, 2008; Oncogene 26: 1616-1625, 2007).

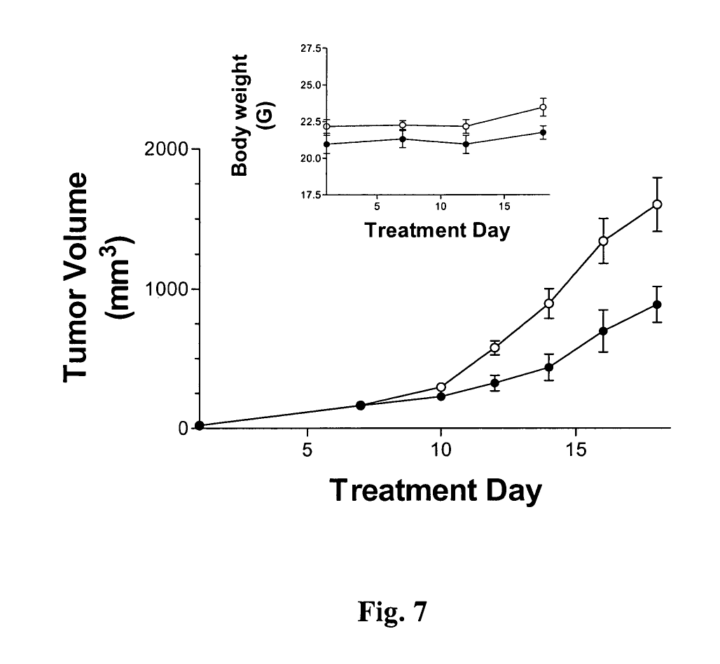

To further evaluate the role of combined treatment of rapamycin and benzylidene-thiazolidine-2,4-dione inhibitors, we have used the FDCP-1 cell line which is IL-3 dependent. To evaluate the effects of D5 and rapamycin, these cells were starved of serum and IL-3 for 1 h during which rapamycin (20 nM), D5 at various concentrations, or a combination of both agents was added. At the end of this incubation, IL-3 was added to stimulate 4E-BP1 phosphorylation. The cells were centrifuged and extracts subjected to SDS-PAGE and immunoblotting. Using an antibody to the Thr37/46 phosphorylation site of 4E-BP1, increasing D5 concentrations reduced the level of the most highly phosphorylated form of 4EBP1 (FIG. 7A, upper arrow) and when combined with rapamycin also decreased the less phosphorylated forms of 4E-BP1 (FIG. 7A, lower arrow). This combined effect is seen in the 4E-BP1 blot as an increase in the lower band. Similar regulation of 4E-BP1 phosphorylation was seen with MV7;11 cells (data not shown). Furthermore, the combined treatment of rapamycin with D5 or 5-(4-iso-propylbenzylidene)thiazolidine-2,4-dione for 72 hours caused significant growth inhibition of MV7;11 and FDCP1 cells with 5-(4-iso-propylbenzylidene)thiazolidine-2,4-dione showing slightly more combined inhibitory effect than D5 (FIG. 7B). To examine the potential synergistic growth inhibitory effect between these D5 or 5-(4-iso-propylbenzyl-idene)thiazolidine-2,4-dione and rapamycin in MV7;11 cells, a combination index analysis was carried out (FIG. 7C). These results demonstrate that at low doses of benzylidene-thiazolidine-2,4-diones and rapamycin the combined effect of these agents is highly synergistic, while at higher concentrations of D5 and 5-(4-iso-propylbenzylidene)thiazolidine-2,4-dione this synergism is lost.

To evaluate the antitumor activity of a Pim inhibitor, 5-(4-iso-propylbenzylidene)-thiazolidine-2,4-dione was administered to Balb/c mice bearing tumors of JC murine mammary adenocarcinoma cells. As indicated in FIG. 8, treatment of the animals with 5-(4-iso-propylbenzylidene)thiazolidine-2,4-dione for 5 days per week did not cause a loss of body weight, consistent with the toxicology studies described above. However, the compound reduced the growth of tumors by approximately 50%. Therefore, the Pim inhibitors of this chemotype have good potential for use as anticancer agents.

The present disclosure relates to methods of treating hyperproliferative diseases. More particularly, the present disclosure relates to a method of treating hyperproliferative diseases, such as cancer. A first embodiment relates to a method for treating a hyperproliferative disease, comprising administering to a human an effective amount of one or more Pim-1 inhibitors as disclosed herein.

Another embodiment relates to a method for treating cancer, comprising administering to a human an effective amount of one or more Pim-1 inhibitors as disclosed herein.

A further embodiment relates to a method for treating cancer, wherein the cancer is chosen from brain, squamous cell, bladder, gastric, pancreatic, breast, head, neck, oesophageal, prostate, colorectal, lung, renal, kidney, ovarian, gynecological and thyroid cancer, comprising administering to a human an effective amount of one or more Pim-1 inhibitors as disclosed herein.

A yet further embodiment relates to a method for treating cancer, comprising administering to a human an effective amount of one or more Pim-1 inhibitors as disclosed herein.

A still further embodiment relates to a method for treating hyperproliferative diseases comprising administering to a human, either simultaneously or sequentially,
a) a therapeutically effective amount of one or more Pim-1 and/or Pim-2 inhibitors as disclosed herein; and
b) an effective amount of one or more mTOR inhibitors;
   wherein if the administered sequentially, the administration can be in any order.

Another further embodiment relates to a method for treating cancer, wherein the cancer is chosen from brain, squamous cell, bladder, gastric, pancreatic, breast, head, neck, oesophageal, prostate, colorectal, lung, renal, kidney, ovarian, gynecological and thyroid cancer, comprising administering to a human, either simultaneously or sequentially,
a) a therapeutically effective amount of one or more Pim-1 and/or Pim-2 inhibitors as disclosed herein; and
b) an effective amount of one or more mTOR inhibitors;
   wherein if the administered sequentially, the administration can be in any order.

Another further embodiment relates to a method for treating prostate cancer, comprising administering to a human, either simultaneously or sequentially,
a) a therapeutically effective amount of one or more Pim-1 and/or Pim-2 inhibitors as disclosed herein; and
b) an effective amount of one or more mTOR inhibitors;
   wherein if the administered sequentially, the administration can be in any order.

A still further embodiment relates to a method for treating hyperproliferative diseases comprising administering to a human, either simultaneously or sequentially,
a) a therapeutically effective amount of one or more Pim-1 inhibitors as disclosed herein; and
b) an effective amount of rapamycin;
   wherein if the administered sequentially, the administration can be in any order.

Another further embodiment relates to a method for treating cancer, wherein the cancer is chosen from brain, squamous cell, bladder, gastric, pancreatic, breast, head, neck, oesophageal, prostate, colorectal, lung, renal, kidney, ovarian, gynecological and thyroid cancer, comprising administering to a human, either simultaneously or sequentially,
a) a therapeutically effective amount of one or more Pim-1 and/or Pim-2 inhibitors as disclosed herein; and
b) an effective amount of rapamycin;
   wherein if the administered sequentially, the administration can be in any order.

Another further embodiment relates to a method for treating prostate cancer, comprising administering to a human, either simultaneously or sequentially,
a) a therapeutically effective amount of one or more Pim-1 and/or Pim-2 inhibitors as disclosed herein; and
b) an effective amount of rapamycin;
   wherein if the administered sequentially, the administration can be in any order.

A still further embodiment relates to a method for treating hyperproliferative diseases comprising administering to a human, either simultaneously or sequentially,
a) a therapeutically effective amount of one or more Pim-1 inhibitors as disclosed herein; and
b) an effective amount of PKC412;
   wherein if the administered sequentially, the administration can be in any order.

A another further embodiment relates to a method for treating cancer, wherein the cancer is chosen from brain, squamous cell, bladder, gastric, pancreatic, breast, head, neck, oesophageal, prostate, colorectal, lung, renal, kidney, ovarian, gynecological and thyroid cancer, comprising administering to a human, either simultaneously or sequentially,
a) a therapeutically effective amount of one or more Pim-1 and/or Pim-2 inhibitors as disclosed herein; and
b) an effective amount of PKC412;
   wherein if the administered sequentially, the administration can be in any order.

Another further embodiment relates to a method for treating prostate cancer, comprising administering to a human, either simultaneously or sequentially,
a) a therapeutically effective amount of one or more Pim-1 and/or Pim-2 inhibitors as disclosed herein; and
b) an effective amount of PKC412;
   wherein if the administered sequentially, the administration can be in any order.

A yet further embodiment relates to a method for treating a non-cancerous hyperproliferative disorder, for example, benign hyperplasia of the skin (e.g., psoriasis) or prostate (e.g., benign prostatic hypertrophy (BPH)).

The present disclosure relates to the use of the disclosed compounds for making a medicament for treating hyperproliferative diseases. More particularly, the present disclosure relates to the use of the disclosed compounds for making a medicament for treating hyperproliferative diseases, such as cancer.

Another embodiment relates to the use of a disclosed compound for treating cancer, comprising administering to a human an effective amount of one or more Pim-1 inhibitors as disclosed herein.

A further embodiment relates to the use of a compound for making a medicament for treating a cancer chosen from brain, squamous cell, bladder, gastric, pancreatic, breast, head, neck, oesophageal, prostate, colorectal, lung, renal, kidney, ovarian, gynecological and thyroid cancer.

A still further embodiment relates to the use of a medicament for treating hyperproliferative diseases comprising:
a) a therapeutically effective amount of one or more Pim-1 and/or Pim-2 inhibitors as disclosed herein; and
b) an effective amount of one or more mTOR inhibitors;
   wherein the medicament can be administered to a human, either simultaneously or sequentially, and wherein if the medicament is administered sequentially, the administration can be in any order.

Another further embodiment relates to the use of a medicament for treating cancer, wherein the cancer is chosen from brain, squamous cell, bladder, gastric, pancreatic, breast, head, neck, oesophageal, prostate, colorectal, lung, renal, kidney, ovarian, gynecological and thyroid cancer:
a) a therapeutically effective amount of one or more Pim-1 and/or Pim-2 inhibitors as disclosed herein; and
b) an effective amount of one or more mTOR inhibitors;
   wherein if the administered sequentially, the administration can be in any order and wherein if the medicament is administered sequentially, the administration can be in any order.

Another further embodiment relates to the use of a combination of medicaments for treating prostate cancer, comprising:
a) a therapeutically effective amount of one or more Pim-1 and/or Pim-2 inhibitors as disclosed herein; and
b) an effective amount of one or more mTOR inhibitors;
   wherein if the administered sequentially, the administration can be in any order and wherein if the medicament is administered sequentially, the administration can be in any order.

A still further embodiment relates to the use of a combination of medicaments for treating hyperproliferative diseases comprising:
a) a therapeutically effective amount of one or more Pim-1 and/or Pim-2 inhibitors as disclosed herein; and
b) an effective amount of rapamycin;
   wherein if the administered sequentially, the administration can be in any order.

Another further embodiment relates to the use of a combination of medicaments for treating cancer, wherein the cancer is chosen from brain, squamous cell, bladder, gastric, pancreatic, breast, head, neck, oesophageal, prostate, colorectal, lung, renal, kidney, ovarian, gynecological and thyroid cancer, comprising:
a) a therapeutically effective amount of one or more Pim-1 and/or Pim-2 inhibitors as disclosed herein; and
b) an effective amount of rapamycin;
   wherein if the administered sequentially, the administration can be in any order.

A still further embodiment relates to the use of a combination of medicaments for treating hyperproliferative diseases comprising:
a) a therapeutically effective amount of one or more Pim-1 and/or Pim-2 inhibitors as disclosed herein; and
b) an effective amount of PKC412;
   wherein if the administered sequentially, the administration can be in any order.

Another further embodiment relates to the use of a combination of medicaments for treating cancer, wherein the cancer is chosen from brain, squamous cell, bladder, gastric, pancreatic, breast, head, neck, oesophageal, prostate, colorectal, lung, renal, kidney, ovarian, gynecological and thyroid cancer, comprising:
a) a therapeutically effective amount of one or more Pim-1 and/or Pim-2 inhibitors as disclosed herein; and
b) an effective amount of PKC412;
   wherein if the administered sequentially, the administration can be in any order.
   A yet further embodiment relates to the use of a disclosed inhibitor for stimulating the phosphorylation of multiple substrates of AMPK in vivo, in vitro, or ex vitro.

### FORMULATIONS

The present disclosure also relates to compositions or formulations which comprise the Pim-1 inhibitors according to the present disclosure. The compositions of the present disclosure comprise:
a) a therapeutically effective amount of one or more Pim-1 and/or Pim-2 inhibitors according to the present disclosure; and
b) one or more pharmaceutically acceptable excipients.

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present disclosure have improved cellular potency, pharmacokinetic properties, as well as improved oral bioavailability.

Non-limiting examples of compositions according to the present disclosure include:
a) from about 0.001 mg to about 1000 mg of one or more Pim-1 and/or Pim-2 inhibitors according to the present disclosure; and
b) one or more pharmaceutically acceptable excipients.

Another example according to the present disclosure relates to the following compositions:
a) from about 0.01 mg to about 100 mg of one or more Pim-1 and/or Pim-2 inhibitors according to the present disclosure; and
b) one or more pharmaceutically acceptable excipients.

A further example according to the present disclosure relates to the following compositions:
a) from about 0.1 mg to about 10 mg of one or more Pim-1 and/or Pim-2 inhibitors according to the present disclosure; and
b) one or more pharmaceutically acceptable excipients.

The disclosure also relates to combination therapies, for example, a pharmaceutical composition comprising one or more pharmaceutically active compounds in combination with one or more Pim-1 inhibitors. One embodiment relates to compositions comprising:
a) a therapeutically effective amount of one or more Pim-1 and/or Pim-2 inhibitors according to the present disclosure; and
b) an effective amount of rapamycin.

A non-limiting example of an mTOR inhibitor is rapamycin. Rapamycin (also known as sirolimus) is marketed under the trade name RAPAMUNE™ by Wyeth. The chemical name for rapamycin is (3S,6R,7E,9R,10R,12R,14S,-15E,17E,19E,21S,23S,-26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29 (4H,6H,31H)-pentone.

FIG. 7 shows the effect of various Pim-1 inhibitors disclosed herein on MV7;11 cells (human leukemic cell line containing the FLT3/ITD mutation). The cells were treated with 5 μM of the captioned Pim inhibitor (from Table A above) alone (black bars) or in combination with 5 nM rapamycin and the cell survival was measured at 72 hour. The results are shown as a percentage normalized to survival of cell treated with 0.2% DMSO. A National Cancer Institute (compound NCI-237538) reference and doxorubicin, a chemotherapy drug, were tested concurrently with the samples.

Another example according to the present disclosure relates to the following compositions:
a) from about 0.01 mg to about 100 mg of one or more Pim-1 and/or Pim-2 inhibitors according to the present disclosure;
b) an effective amount of rapamycin; and
c) one or more excipients.

A further example according to the present disclosure relates to the following compositions:
a) from about 0.1 mg to about 10 mg of one or more human protein Pim-1 and/or Pim-2 inhibitors according to the present disclosure;
b) an effective amount of rapamycin; and
c) one or more excipients.

A further embodiment relates to compositions comprising:
a) a therapeutically effective amount of one or more Pim-1 and/or Pim-2 inhibitors according to the present disclosure; and
b) an effective amount of PKC412.
   PCK412 is N-benzoyl staurosporine. The chemical name for PCK412 is 4′-N-benzoyl-(9S,10R,11R,13R)-2,3,10,11,12,13-Hexahydro-10-methoxy-9-methyl-11-(methylamino)-9,13-epoxy-1H,9H-diindolo[1,2,3-gh:3′,2′,1′-lm]pyrrolo[3,4-j][1,7]benzodiazonin-1-one having the formula:

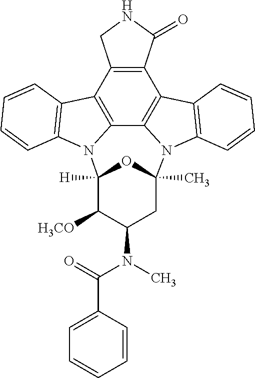

which is available from LC Laboratories a division of LCK Pharmaceuticals

Another example according to the present disclosure relates to the following compositions:
a) from about 0.01 mg to about 100 mg of one or more Pim-1 and/or Pim-2 inhibitors according to the present disclosure;
b) an effective amount of PKC412; and
c) one or more excipients.

A further example according to the present disclosure relates to the following compositions:
a) from about 0.1 mg to about 10 mg of one or more human protein Pim-1 and/or Pim-2 inhibitors according to the present disclosure;
b) an effective amount of PKC412; and
c) one or more excipients.

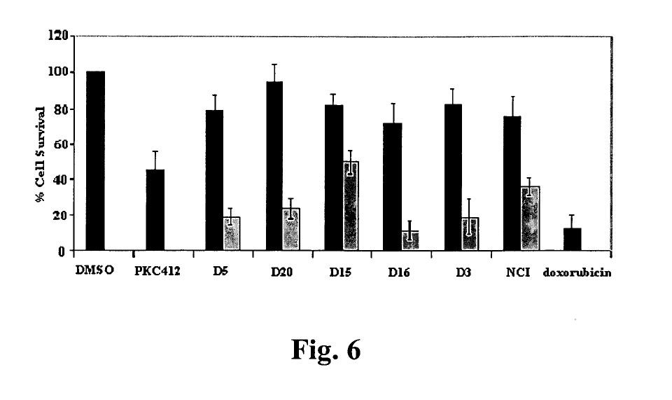

FIG. 6 shows the effect of various Pim-1 inhibitors disclosed herein on MV7;11 cells (human leukemic cell line containing the FLT3/ITD mutation). The cells were treated with 5 μM of the captioned Pim inhibitor (from Table A above) alone (black bars) or in combination with 5 nM PKC412 and the cell survival was measured at 72 hour. The results are shown as a percentage normalized to survival of cell treated with 0.2% DMSO. A National Cancer Institute (compound NCI-237538) reference and doxorubicin, a chemotherapy drug, were tested concurrently with the samples.

The term “therapeutically effective amount” as used herein means “an amount of one or more Pim-1 inhibitors, effective at dosages and for periods of time necessary to achieve the desired or therapeutic result.” An effective amount may vary according to factors known in the art, such as the disease state, age, sex, and weight of the human or animal being treated. Although particular dosage regimes may be described in examples herein, a person skilled in the art would appreciate that the dosage regime may be altered to provide optimum therapeutic response. For example, several divided doses may be administered daily or the dose may be proportionally reduced as indicated by the exigencies of the therapeutic situation. In addition, the compositions of the present disclosure can be administered as frequently as necessary to achieve a therapeutic amount.

As described herein above, the formulations of the present disclosure include pharmaceutical compositions comprising a compound that can inhibit the activity of Pim-1 and/or Pim-2 and therefore is suitable for use in treating cancer, non-limiting examples of which include brain, squamous cell, bladder, gastric, pancreatic, breast, head, neck, oesophageal, prostate, colorectal, lung, renal, kidney, ovarian, gynecological and thyroid cancer, and other hyperproliferative diseases and a pharmaceutically-acceptable carrier, vehicle, or diluent. Those skilled in the art based upon the present description and the nature of any given inhibitor identified by the assays of the present disclosure will understand how to determine a therapeutically effective dose thereof.

The pharmaceutical compositions may be manufactured using any suitable means, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically or pharmaceutically acceptable carriers (vehicles, or diluents) comprising excipients and auxiliaries which facilitate processing of the active compounds into preparations which can be used pharmaceutically. Proper formulation is dependent upon the route of administration chosen.

Any suitable method of administering a pharmaceutical composition to a patient may be used in the methods of treatment of the present disclosure, including injection, transmucosal, oral, inhalation, ocular, rectal, long acting implantation, liposomes, emulsion, or sustained release means.

For injection, the agents of the present disclosure may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. For ocular administration, suspensions in an appropriate saline solution are used as is well known in the art.

For oral administration, the compounds can be formulated readily by combining the active compounds with pharmaceutically acceptable carriers well known in the art. Such carriers enable the compounds of the present disclosure to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, such as sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

One type of pharmaceutical carrier for hydrophobic compounds of the present disclosure is a cosolvent system comprising benzyl alcohol, a nonpolar surfactant, a water-miscible organic polymer, and an aqueous phase.

The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed.

Additionally, the compounds may be delivered using any suitable sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a prolonged period of time. Depending on the chemical nature and the biological stability of the therapeutic reagent, additional strategies for compound stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the agents of the present disclosure may be provided as salts with pharmaceutically acceptable counterions. Salts tend to be more soluble in aqueous or other protic solvents than are the corresponding free base forms.

Other aspects of the present disclosure include methods of treating a condition or a disease in a mammal comprising administering to said mammal a pharmaceutical composition of the present disclosure.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure. All cited references are included herein by reference in their entirety.

1. The use of a compound for treating cancer in a human, comprising administering to a human an effective amount of one or more compounds, or a pharmaceutically acceptable salt thereof, having the formula: 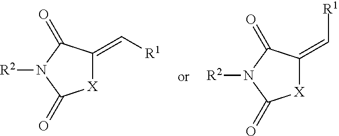
wherein
X is S or NR³;
R³ is benzyl or benzyl substituted by from 1 to 5 organic radicals;
R¹ is phenyl or phenyl substituted by from 1 to 5 organic radicals; and
R² is chosen from:
i) hydrogen;
ii) C₁-C₄ linear, branched, or cyclic alkyl; and
iii) benzyl or benzyl substituted by from 1 to 5 organic radicals.

2. The use of a compound for treating cancer in a human, comprising administering to a human an effective amount of one or more compounds, or a pharmaceutically acceptable salt thereof, having the formula: 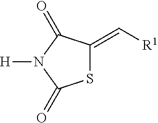
wherein R¹ is phenyl or phenyl substituted by from 1 to 5 organic radicals.

3. The use according to claim 2, wherein the compound has the formula: 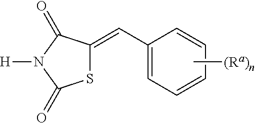
the index n is from 1 to 5;
R^{a} is from 1 to 5 organic radicals that are substitutions for hydrogen independently chosen from:
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{4a})(R^{4b})]_{y}OR⁵;
wherein R⁵ is chosen from:
a) —H;
b) C₁-C₁₂ substituted or unsubstituted linear, branched, or cyclic alkyl or C₁-C₁₂ substituted or unsubstituted linear, branched, or cyclic haloalkyl;
c) C₆ or C₁₀ substituted or unsubstituted aryl or C₇-C₂₀ alkylenearyl;
d) C₁-C₉ substituted or unsubstituted heterocyclic; and
e) C₁-C₁₁ substituted or unsubstituted heteroaryl;
viii) —[C(R^{4a})(R^{4b})]_{y}N(R^{6a})(R^{6b});
wherein R^{6a} and R^{6b} are each independently chosen from:
a) —H;
b) —OR⁷;
R⁷ is hydrogen or C₁-C₄ linear alkyl;
c) C₁-C₁₂ substituted or unsubstituted linear, branched, or cyclic alkyl;
d) C₆ or C₁₀ substituted or unsubstituted aryl;
e) C₁-C₉ substituted or unsubstituted heterocyclic;
f) C₁-C₁₁ substituted or unsubstituted heteroaryl; and
g) R^{6a} and R^{6b} can be taken together to form a substituted or unsubstituted ring having from 3 to 10 carbon atoms and from 0 to 3 heteroatoms chosen from oxygen, nitrogen, and sulfur;
ix) —[C(R^{4a})(R^{4b})]_{y}C(O)R⁸;
wherein R⁸ is chosen from:
a) C₁-C₁₂ substituted or unsubstituted linear, branched, or cyclic alkyl;
b) —OR⁹;
wherein R⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
c) —N(R^{10a})(R^{10b});
wherein R^{10a} and R^{10b} are each independently hydrogen, C₁-C₁₂ substituted or unsubstituted linear, branched, or cyclic alkyl; C₆ or C-₁₀ substituted or unsubstituted aryl; C₁-C₉ substituted or unsubstituted heterocyclic; C₁-C₁₁ substituted or unsubstituted heteroaryl; or R^{10a} and R^{10b} can be taken together to form a substituted or unsubstituted ring having from 3 to 10 carbon atoms and from 0 to 3 heteroatoms chosen from oxygen, nitrogen, and sulfur;
x) —[C(R^{4a})(R^{4b})]_{y}OC(O)R¹¹;
wherein R¹¹ is chosen from:
a) C₁-C₁₂ substituted or unsubstituted linear, branched, or cyclic alkyl; and)
b) —N(R^{12a})(R^{12b});
R^{12a} and R^{12b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{12a} and R^{12b} can be taken together to form a substituted or unsubstituted ring having from 3 to 10 carbon atoms and from 0 to 3 heteroatoms chosen from oxygen, nitrogen, and sulfur;
xi) —[C(R^{4a})(R^{4b})]_{y}NR¹³C(O)R¹⁴;
wherein R¹³ is chosen from:
a) —H; and
b) C₁-C₄ substituted or unsubstituted linear, branched, or cyclic alkyl;
wherein R¹⁴ is chosen from:
a) C₁-C₁₂ substituted or unsubstituted linear, branched, or cyclic alkyl; and
b) —N(R^{15a})(R^{15b});
R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
xii) —[C(R^{4a})(R^{4b})]_{y}CN;
xiii) —[C(R^{4a})(R^{4b})]_{y}NO₂;
xiv) —[C(R^{4a})(R^{4b})]_{y}SO₂R¹⁶;
R¹⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl; and
xv) halogen;
R^{4a} and R^{4b} are each independently hydrogen or C₁-C₄ alkyl; and
the index y is from 0 to 5.

4. The use according to claim 3, wherein each R^{a} is an organic radical independently chosen from:
i) C₁-C₄ linear, branched, or cyclic alkyl;
ii) C₁-C₄ haloalkyl;
iii) phenyl;
iv) —OR⁵;
wherein R⁵ is chosen from:
a) —H; and
b) C₁-C₄ linear or branched alkyl;
v) —N(R^{6a})(R^{6b});
wherein R^{6a} and R^{6b} are each independently chosen from:
a) —H; and
b) C₁-C₄ linear or branched alkyl;
vi) —C(O)R⁸;
wherein R⁸ is chosen from:
a) C₁-C₄ linear or branched alkyl;
b) —OR⁹;
R⁹ is hydrogen or C₁-C₄ linear alkyl; and
c) —N(R^{10a})(R^{10b});
R^{10a} and R^{10b} are each independently hydrogen or C₁-C₄ linear alkyl;
vii) —OC(O)R¹¹; R¹¹ is C₁-C₄ linear or branched alkyl or phenyl;
viii) —CN;
ix) —NO₂;
x) —SO₂R¹⁶; R¹⁶ is hydrogen, hydroxyl, or C₁-C₄ linear or branched alkyl; and
xi) halogen.

5. The use according to claim 3, wherein each R^{a} is an organic radical independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

6. A use according to claim 2,
wherein the compound is chosen from:
5-(3-fluorobenzylidene)thiazolidine-2,4-dione;
5-(3-chlorobenzylidene)thiazolidine-2,4-dione;
5-(3-bromobenzylidene)thiazolidine-2,4-dione;
5-(3-methylbenzylidene)thiazolidine-2,4-dione;
5-(3-trifluoromethylbenzylidene)thiazolidine-2,4-dione;
5-(3-methoxybenzylidene)thiazolidine-2,4-dione;
5-(3-trifluoromethoxybenzylidene)thiazolidine-2,4-dione;
5-[3-(1,1,2,2-tetrafluoroethoxy)benzylidene]thiazolidine-2,4-dione;
5-(4-fluorobenzylidene)thiazolidine-2,4-dione;
5-(4-chlorobenzylidene)thiazolidine-2,4-dione;
5-(4-bromobenzylidene)thiazolidine-2,4-dione;
5-(4-methylbenzylidene)thiazolidine-2,4-dione;
5-(4-ethylbenzylidene)thiazolidine-2,4-dione;
5-(4-methoxybenzylidene)thiazolidine-2,4-dione;
5-(4-ethoxybenzylidene)thiazolidine-2,4-dione;
5-(4-propoxybenzylidene)thiazolidine-2,4-dione;
5-(4-iso-propoxybenzylidene)thiazolidine-2,4-dione;
5-(4-trifluoromethylbenzylidene)thiazolidine-2,4-dione;
5-(4-trifluoromethoxybenzylidene)thiazolidine-2,4-dione;
5-(4-dimethylaminobenzylidene)thiazolidine-2,4-dione; and
5-(4-methylcarboxybenzylidene)thiazolidine-2,4-dione.

7. The use of a compound, or a pharmaceutically acceptable salt thereof, for treating cancer, wherein the cancer is chosen from brain, squamous cell, bladder, gastric, pancreatic, breast, head, neck, oesophageal, prostate, colorectal, lung, renal, kidney, ovarian, gynecological, thyroid cancer, and hematologic cancer comprising administering to a human an effective amount of one or more compounds having the formula: 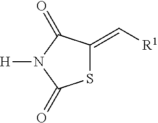
wherein R¹ is phenyl or phenyl substituted by from 1 to 5 organic radicals.

8. The use of a compound for treating a hyperproliferative disease, comprising administering to a human an effective amount of one or more compounds, or a pharmaceutically acceptable salt thereof, having the formula: 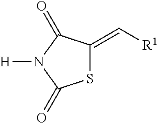
wherein R¹ is phenyl or phenyl substituted by from 1 to 5 organic radicals.

9. The use of a composition for treating cancer, wherein the medicament comprises:
a) a therapeutically effective amount of one or more compounds, or a pharmaceutically acceptable salt thereof, having the formula: 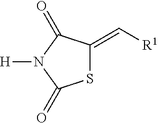
wherein R¹ is phenyl or phenyl substituted by from 1 to 5 organic radicals; and
b) one or more pharmaceutically acceptable carriers.

10. The use of a composition for treating cancer, wherein the medicament comprises:
a) a therapeutically effective amount of one or more compounds, or a pharmaceutically acceptable salt thereof, having the formula: 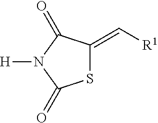
wherein R¹ is phenyl or phenyl substituted by from 1 to 5 organic radicals; and
b) an effective amount of rapamycin.

11. The use of a composition, or a pharmaceutically acceptable salt thereof, for treating cancer, wherein the medicament comprises: 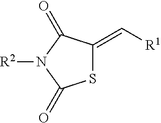
wherein R¹ is phenyl or phenyl substituted by from 1 to 5 organic radicals; and R² is chosen from:
i) hydrogen;
ii) C₁-C₄ linear, branched, or cyclic alkyl; and
iii) benzyl or benzyl substituted by from 1 to 5 organic radicals.

12. The use according to claim 11, wherein the compound has the formula: 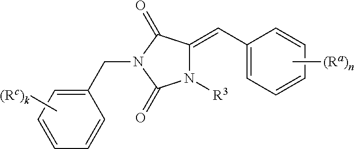
the indices k and n are each independently from 1 to 5;
R^{a} is from 1 to 5 organic radicals that are substitutions for hydrogen independently chosen from:
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{4a})(R^{4b})]_{y}OR⁵;
wherein R⁵ is chosen from:
a) —H;
b) C₁-C₁₂ substituted or unsubstituted linear, branched, or cyclic alkyl or C₁-C₁₂ substituted or unsubstituted linear, branched, or cyclic haloalkyl;
c) C₆ or C₁₀ substituted or unsubstituted aryl or C₇-C₂₀ alkylenearyl;
d) C₁-C₉ substituted or unsubstituted heterocyclic; and
e) C₁-C₁₁ substituted or unsubstituted heteroaryl;
viii) —[C(R^{4a})(R^{4b})]_{y}N(R^{6a})(R^{6b});
wherein R^{6a} and R^{6b} are each independently chosen from:
a) —H;
b) —OR⁷;
R⁷ is hydrogen or C₁-C₄ linear alkyl;
c) C₁-C₁₂ substituted or unsubstituted linear, branched, or cyclic alkyl;
d) C₆ or C₁₀ substituted or unsubstituted aryl;
e) C₁-C₉ substituted or unsubstituted heterocyclic;
f) C₁-C₁₁ substituted or unsubstituted heteroaryl; and
g) R^{6a} and R^{6b} can be taken together to form a substituted or unsubstituted ring having from 3 to 10 carbon atoms and from 0 to 3 heteroatoms chosen from oxygen, nitrogen, and sulfur;
ix) —[C(R^{4a})(R^{4b})]_{y}C(O)R⁸;
wherein R⁸ is chosen from:
a) C₁-C₁₂ substituted or unsubstituted linear, branched, or cyclic alkyl;
b) —OR⁹;
wherein R⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
c) —N(R^{10a})(R^{10b});
wherein R^{10a} and R^{10b} are each independently hydrogen, C₁-C₁₂ substituted or unsubstituted linear, branched, or cyclic alkyl; C₆ or C-₁₀ substituted or unsubstituted aryl; C₁-C₉ substituted or unsubstituted heterocyclic; C₁-C₁₁ substituted or unsubstituted heteroaryl; or R^{10a} and R^{10b} can be taken together to form a substituted or unsubstituted ring having from 3 to 10 carbon atoms and from 0 to 3 heteroatoms chosen from oxygen, nitrogen, and sulfur;
x) —[C(R^{4a})(R^{4b})]_{y}OC(O)R¹¹;
wherein R¹¹ is chosen from:
a) C₁-C₁₂ substituted or unsubstituted linear, branched, or cyclic alkyl; and
b) —N(R^{12a})(R^{12b});
R^{12a} and R^{12b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{12a} and R^{12b} can be taken together to form a substituted or unsubstituted ring having from 3 to 10 carbon atoms and from 0 to 3 heteroatoms chosen from oxygen, nitrogen, and sulfur;
xi) —[C(R^{4a})(R^{4b})]_{y}NR¹³C(O)R¹⁴;
wherein R¹³ is chosen from:
a) —H; and
b) C₁-C₄ substituted or unsubstituted linear, branched, or cyclic alkyl;
wherein R¹⁴ is chosen from:
a) C₁-C₁₂ substituted or unsubstituted linear, branched, or cyclic alkyl; and
b) —N(R^{15a})(R^{15b});
R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
xii) —[C(R^{4a})(R^{4b})]_{y}CN;
xiii) —[C(R^{4a})(R^{4b})]_{y}NO₂;
xiv) —[C(R^{4a})(R^{4b})]_{y}SO₂R¹⁶;
R¹⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl; and
xv) halogen;
R^{4a} and R^{4b} are each independently hydrogen or C₁-C₄ alkyl; and
the index y is from 0 to 5;
each R^{c} is independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

13. The use according to claim 12, wherein each R^{a} is an organic radical independently chosen from:
i) C₁-C₄ linear, branched, or cyclic alkyl;
ii) C₁-C₄ haloalkyl;
iii) phenyl;
iv) —OR⁵;
wherein R⁵ is chosen from:
a) —H; and
b) C₁-C₄ linear or branched alkyl;
v) —N(R^{6a})(R^{6b});
wherein R^{6a} and R^{6b} are each independently chosen from:
a) —H; and
b) C₁-C₄ linear or branched alkyl;
vi) —C(O)R⁸;
wherein R⁸ is chosen from:
a) C₁-C₄ linear or branched alkyl;
b) —OR⁹;
R⁹ is hydrogen or C₁-C₄ linear alkyl; and
c) —N(R^{10a})(R^{10b});
R^{10a} and R^{10b} are each independently hydrogen or C₁-C₄ linear alkyl;
vii) —OC(O)R¹¹; R¹¹ is C₁-C₄ linear or branched alkyl or phenyl;
viii) —CN;
ix) —NO₂;
x) —SO₂R¹⁶; R¹⁶ is hydrogen, hydroxyl, or C₁-C₄ linear or branched alkyl; and
xi) halogen.

14. The uses according to claim 12, wherein each R^{a} is an organic radical independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

15. The use according to claim 12, wherein each R^{a} is an organic radical independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃; and
each R^{c} is an organic radical independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

16. The use of a compound, or a pharmaceutically acceptable salt thereof, for treating cancer, wherein the cancer is chosen from brain, squamous cell, bladder, gastric, pancreatic, breast, head, neck, oesophageal, prostate, colorectal, lung, renal, kidney, ovarian, gynecological, thyroid cancer, and hematologic cancer comprising administering to a human an effective amount of one or more compounds having the formula: 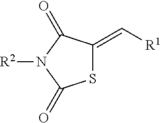
wherein R¹ is phenyl or phenyl substituted by from 1 to 5 organic radicals; and R² is chosen from:
i) hydrogen;
ii) C₁-C₄ linear, branched, or cyclic alkyl; and
iii) benzyl or benzyl substituted by from 1 to 5 organic radicals.

17. The use of a compound for treating a hyperproliferative disease, comprising administering to a human an effective amount of one or more compounds, or a pharmaceutically acceptable salt thereof, having the formula: 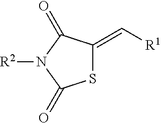
wherein R¹ is phenyl or phenyl substituted by from 1 to 5 organic radicals; and R² is chosen from:
i) hydrogen;
ii) C₁-C₄ linear, branched, or cyclic alkyl; and
iii) benzyl or benzyl substituted by from 1 to 5 organic radicals.

18. The use of a composition for treating cancer, wherein the medicament comprises:
a) a therapeutically effective amount of one or more compounds, or a pharmaceutically acceptable salt thereof, having the formula: 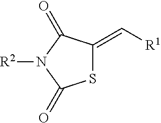
wherein R¹ is phenyl or phenyl substituted by from 1 to 5 organic radicals; and
R² is chosen from:
i) hydrogen;
ii) C₁-C₄ linear, branched, or cyclic alkyl; and
iii) benzyl or benzyl substituted by from 1 to 5 organic radicals; and
b) one or more pharmaceutically acceptable carriers.

19. The use of a composition for treating cancer, wherein the medicament comprises:
a) a therapeutically effective amount of one or more compounds, or a pharmaceutically acceptable salt thereof, having the formula: 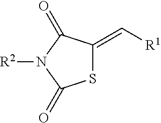
wherein R¹ is phenyl or phenyl substituted by from 1 to 5 organic radicals; and
R² is chosen from:
i) hydrogen;
ii) C₁-C₄ linear, branched, or cyclic alkyl; and
iii) benzyl or benzyl substituted by from 1 to 5 organic radicals; and
b) an effective amount of rapamycin.

20. The use of a compound for treating cancer in a human, comprising administering to a human an effective amount of one or more compounds, or a pharmaceutically acceptable salt thereof, having the formula: 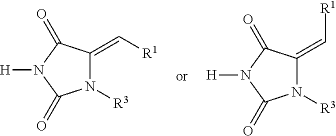
wherein R¹ is phenyl or phenyl substituted by from 1 to 5 organic radicals; and
R³ is benzyl or benzyl substituted by from 1 to 5 organic radicals.

21. A use according to claim 20, wherein the compound has the formula: 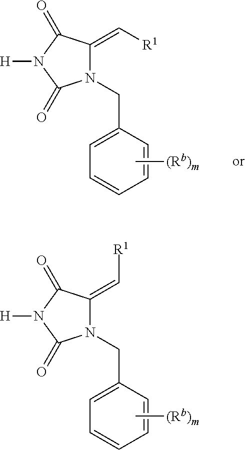
wherein the index m is from 1 to 5;
each R^{b} is independently chosen from:
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₁-C₁₂ substituted or unsubstituted linear or branched haloalkyl;
iv) C₆ or C₁₀ substituted or unsubstituted aryl;
v) C₁-C₉ substituted or unsubstituted heterocyclic; as described herein below;
vi) C₁-C₁₁ substituted or unsubstituted heteroaryl; as described herein below;
vii) —[C(R^{24a})(R^{24b})]_{z}OR²⁵;
wherein R²⁵ is chosen from:
a) —H;
b) C₁-C₁₂ substituted or unsubstituted linear, branched, or cyclic alkyl or C₁-C₁₂ substituted or unsubstituted linear, branched, or cyclic haloalkyl;
c) C₆ or C₁₀ substituted or unsubstituted aryl or C₇-C₂₀ alkylenearyl;
d) C₁-C₉ substituted or unsubstituted heterocyclic; and
e) C₁-C₁₁ substituted or unsubstituted heteroaryl;
viii) —[C(R²⁴a)(R^{24b})]_{z}N(R^{26a})(R^{26b});
wherein R^{26a} and R^{26b} are each independently chosen from:
a) —H;
b) —OR²⁷;
R²⁷ is hydrogen or C₁-C₄ linear alkyl;
c) C₁-C₁₂ substituted or unsubstituted linear, branched, or cyclic alkyl;
d) C₆ or C₁₀ substituted or unsubstituted aryl;
e) C₁-C₉ substituted or unsubstituted heterocyclic;
f) C₁-C₁₁ substituted or unsubstituted heteroaryl; and
g) R^{26a} and R^{26b} can be taken together to form a substituted or unsubstituted ring having from 3 to 10 carbon atoms and from 0 to 3 heteroatoms chosen from oxygen, nitrogen, and sulfur;
ix) —[C(R^{24a})(R^{24b})]_{z}C(O)R²⁸;
wherein R²⁸ is chosen from:
a) C₁-C₁₂ substituted or unsubstituted linear, branched, or cyclic alkyl;
b) —OR²⁹;
R²⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
c) —N(R^{30a})(R^{30b});
R^{30a} and R^{30b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{30a} and R^{30b} can be taken together to form a substituted or unsubstituted ring having from 3 to 10 carbon atoms and from 0 to 3 heteroatoms chosen from oxygen, nitrogen, and sulfur;
x) —[C(R^{24a})(R^{24b})]_{z}OC(O)R³¹;
wherein R³¹ is chosen from:
a) C₁-C₁₂ substituted or unsubstituted linear, branched, or cyclic alkyl; and
b) —N(R^{32a})(R^{32b});
R^{32a} and R^{32b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{32a} and R^{32b} can be taken together to form a substituted or unsubstituted ring having from 3 to 10 carbon atoms and from 0 to 3 heteroatoms chosen from oxygen, nitrogen, and sulfur;
xi) —[C(R^{24a})(R^{24b})]_{z}NR³³C(O)R³⁴;
wherein R³³ is chosen from:
a) —H; and
b) C₁-C₄ substituted or unsubstituted linear, branched, or cyclic alkyl;
wherein R³⁴ is chosen from:
a) C₁-C₁₂ substituted or unsubstituted linear, branched, or cyclic alkyl; and
b) —N(R^{35a})(R^{35b});
R^{35a} and R^{35b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{35a} and R^{35b} can be taken together to form a substituted or unsubstituted ring having from 3 to 10 carbon atoms and from 0 to 3 heteroatoms chosen from oxygen, nitrogen, and sulfur;
xii) —[C(R^{24a})(R^{24b})]_{z}CN;
xiii) —[C(R^{24a})(R^{24b})]_{z}NO₂;
xiv) —[C(R^{24a})(R^{24b})]_{z}SO₂R³⁶;
R³⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl; and
xv) halogen; —F, —Cl, —Br, and —I;
R^{24a} and R^{24b} are each independently hydrogen or C₁-C₄ alkyl; and
the index z is from 0 to 6.

22. The use according to claim 21, wherein the compound has the formula: 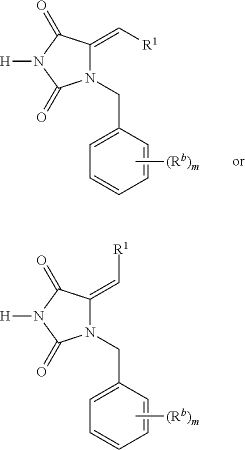
wherein the index m is from 1 to 5;
each R^{b} is independently chosen from:
i) C₁-C₄ linear, branched, or cyclic alkyl;
ii) C₁-C₄ haloalkyl;
iii) phenyl;
iv) —OR²⁵;
wherein R²⁵ is chosen from:
a) —H; and
b) C₁-C₄ linear or branched alkyl or C₁-C₄ linear or branched haloalkyl;
v) —N(R^{26a})(R^{26b});
wherein R^{26a} and R^{26b} are each independently chosen from:
a) —H; and
b) C₁-C₄ linear or branched alkyl;
vi) —C(O)R²⁸;
wherein R²⁸ is chosen from:
a) C₁-C₄ linear or branched alkyl;
b) —OR²⁸;
R²⁸ is hydrogen or C₁-C₄ linear alkyl; and
c) —N(R^{30a})(R^{30b});
R^{30a} and R^{30b} are each independently hydrogen or C₁-C₄ linear alkyl;
vii) —OC(O)R³¹; R³¹ is C₁-C₄ linear or branched alkyl or phenyl;
viii) —CN;
ix) —NO₂;
x) —SO₂R³⁶; R³⁶ is hydrogen, hydroxyl, or C₁-C₄ linear or branched alkyl; and
xi) halogen.

23. A use according to claim 21, wherein the compound has the formula: 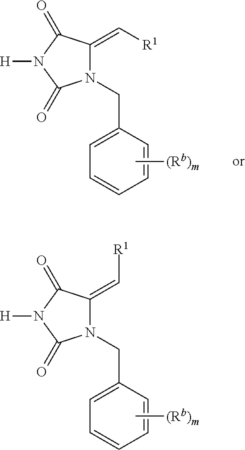
wherein the index m is from 1 to 5;
each R^{b} is independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

24. The use according to claim 21, wherein the compound has the formula: 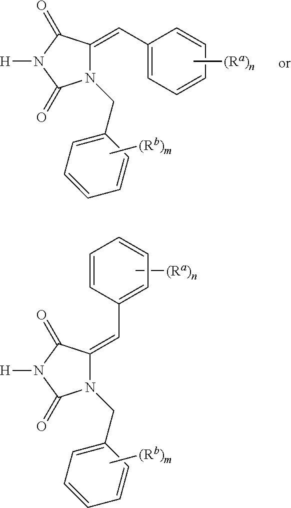
wherein the indices m and n are each independently from 1 to 5;
R^{a} is from 1 to 5 organic radicals that are substitutions for hydrogen independently chosen from:
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{4a})(R^{4b})]_{y}OR⁵;
wherein R⁵ is chosen from:
a) —H;
b) C₁-C₁₂ substituted or unsubstituted linear, branched, or cyclic alkyl or C₁-C₁₂ substituted or unsubstituted linear, branched, or cyclic haloalkyl;
c) C₆ or C₁₀ substituted or unsubstituted aryl or C₇-C₂₀ alkylenearyl;
d) C₁-C₉ substituted or unsubstituted heterocyclic; and
e) C₁-C₁₁ substituted or unsubstituted heteroaryl;
viii) —[C(R^{4a})(R^{4b})]_{y}N(R^{6a})(R^{6b});
wherein R^{6a} and R^{6b} are each independently chosen from:
a) —H;
b) —OR⁷;
R⁷ is hydrogen or C₁-C₄ linear alkyl;
c) C₁-C₁₂ substituted or unsubstituted linear, branched, or cyclic alkyl;
d) C₆ or C₁₀ substituted or unsubstituted aryl;
e) C₁-C₉ substituted or unsubstituted heterocyclic;
f) C₁-C₁₁ substituted or unsubstituted heteroaryl; and
g) R^{6a} and R^{6b} can be taken together to form a substituted or unsubstituted ring having from 3 to 10 carbon atoms and from 0 to 3 heteroatoms chosen from oxygen, nitrogen, and sulfur;
ix) —[C(R^{4a})(R^{4b})]_{y}C(O)R⁸;
wherein R⁸ is chosen from:
a) C₁-C₁₂ substituted or unsubstituted linear, branched, or cyclic alkyl;
b) —OR⁹;
wherein R⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
c) —N(R^{10a})(R^{10b});
wherein R^{10a} and R^{10b} are each independently hydrogen, C₁-C₁₂ substituted or unsubstituted linear, branched, or cyclic alkyl; C₆ or C-₁₀ substituted or unsubstituted aryl; C₁-C₉ substituted or unsubstituted heterocyclic; C₁-C₁₁ substituted or unsubstituted heteroaryl; or R^{10a} and R^{10b} can be taken together to form a substituted or unsubstituted ring having from 3 to 10 carbon atoms and from 0 to 3 heteroatoms chosen from oxygen, nitrogen, and sulfur;
x) —[C(R^{4a})(R^{4b})]_{y}OC(O)R¹¹;
wherein R¹¹ is chosen from:
a) C₁-C₁₂ substituted or unsubstituted linear, branched, or cyclic alkyl; and
b) —N(R^{12a})(R^{12b});
R^{12a} and R^{12b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{12a} and R^{12b} can be taken together to form a substituted or unsubstituted ring having from 3 to 10 carbon atoms and from 0 to 3 heteroatoms chosen from oxygen, nitrogen, and sulfur;
xi) —[C(R^{4a})(R^{4b})]_{y}NR¹³C(O)R¹⁴;
wherein R¹³ is chosen from:
a) —H; and
b) C₁-C₄ substituted or unsubstituted linear, branched, or cyclic alkyl;
wherein R¹⁴ is chosen from:
a) C₁-C₁₂ substituted or unsubstituted linear, branched, or cyclic alkyl; and
b) —N(R^{15a})(R^{15b});
R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
xii) —[C(R^{4a})(R^{4b})]_{y}CN;
xiii) —[C(R^{4a})(R^{4b})]_{y}NO₂;
xiv) —[C(R^{4a})(R^{4b})]_{y}SO₂R¹⁶;
R¹⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl; and
xv) halogen;
R^{4a} and R^{4b} are each independently hydrogen or C₁-C₄ alkyl; and
the index y is from 0 to 5; and
each R^{b} is independently chosen from:
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₁-C₁₂ substituted or unsubstituted linear or branched haloalkyl;
iv) C₆ or C₁₀ substituted or unsubstituted aryl;
v) C₁-C₉ substituted or unsubstituted heterocyclic; as described herein below;
vi) C₁-C₁₁ substituted or unsubstituted heteroaryl; as described herein below;
vii) —[C(R^{24a})(R^{24b})]_{z}OR²⁵;
wherein R²⁵ is chosen from:
a) —H;
b) C₁-C₁₂ substituted or unsubstituted linear, branched, or cyclic alkyl or C₁-C₁₂ substituted or unsubstituted linear, branched, or cyclic haloalkyl;
c) C₆ or C₁₀ substituted or unsubstituted aryl or C₇-C₂₀ alkylenearyl;
d) C₁-C₉ substituted or unsubstituted heterocyclic; and
e) C₁-C₁₁ substituted or unsubstituted heteroaryl;
viii) —[C(R^{24a})(R^{24b})]_{z}N(R^{26a})(R^{26b});
wherein R^{26a} and R^{26b} are each independently chosen from:
a) —H;
b) —OR²⁷;
R²⁷ is hydrogen or C₁-C₄ linear alkyl;
c) C₁-C₁₂ substituted or unsubstituted linear, branched, or cyclic alkyl;
d) C₆ or C₁₀ substituted or unsubstituted aryl;
e) C₁-C₉ substituted or unsubstituted heterocyclic;
f) C₁-C₁₁ substituted or unsubstituted heteroaryl; and
g) R^{26a} and R^{26b} can be taken together to form a substituted or unsubstituted ring having from 3 to 10 carbon atoms and from 0 to 3 heteroatoms chosen from oxygen, nitrogen, and sulfur;
ix) —[C(R^{24a})(R^{24b})]_{z}C(O)R²⁵;
wherein R²⁸ is chosen from:
a) C₁-C₁₂ substituted or unsubstituted linear, branched, or cyclic alkyl;
b) —OR²⁹;
R²⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
c) —N(R^{30a})(R^{30b});
R^{30a} and R^{30b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{30a} and R^{30b} can be taken together to form a substituted or unsubstituted ring having from 3 to 10 carbon atoms and from 0 to 3 heteroatoms chosen from oxygen, nitrogen, and sulfur;
x) —[C(R^{24a})(R^{24b})]_{z}OC(O)R³¹;
wherein R³¹ is chosen from:
a) C₁-C₁₂ substituted or unsubstituted linear, branched, or cyclic alkyl; and
b) —N(R^{32a})(R^{32b});
R^{32a} and R^{32b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{32a} and R^{32b} can be taken together to form a substituted or unsubstituted ring having from 3 to 10 carbon atoms and from 0 to 3 heteroatoms chosen from oxygen, nitrogen, and sulfur;
ix) —[C(R^{24a})(R^{24b})]_{z}NR³³C(O)R³⁴;
wherein R³³ is chosen from:
a) —H; and
b) C₁-C₄ substituted or unsubstituted linear, branched, or cyclic alkyl;
wherein R³⁴ is chosen from:
a) C₁-C₁₂ substituted or unsubstituted linear, branched, or cyclic alkyl; and
b) —N(R^{35a})(R^{35b});
R^{35a} and R^{35b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{35a} and R^{35b} can be taken together to form a substituted or unsubstituted ring having from 3 to 10 carbon atoms and from 0 to 3 heteroatoms chosen from oxygen, nitrogen, and sulfur;
xii) —[C(R^{24a})(R^{24b})]_{z}CN;
xiii) —[C(R^{24a})(R^{24b})]_{z}NO₂;
xiv) —[C(R^{24a})(R^{24b})]_{z}SO₂R³⁶;
R³⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl; and
xv) halogen; —F, —Cl, —Br, and —I;
R^{24a} and R^{24b} are each independently hydrogen or C₁-C₄ alkyl; and
the index z is from 0 to 6.

25. The use according to claim 21, wherein the compound has the formula: 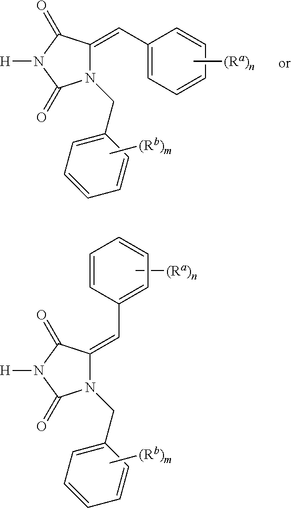
wherein the indices m and n are each independently from 1 to 5;
each R^{a} is an organic radical independently chosen from:
i) C₁-C₄ linear, branched, or cyclic alkyl;
ii) C₁-C₄ haloalkyl;
iii) phenyl;
iv) —OR⁵;
wherein R⁵ is chosen from:
a) —H; and
b) C₁-C₄ linear or branched alkyl;
v) —N(R^{6a})(R^{6b});
wherein R^{6a} and R^{6b} are each independently chosen from:
a) —H; and
b) C₁-C₄ linear or branched alkyl;
vi) —C(O)R⁸;
wherein R⁸ is chosen from:
a) C₁-C₄ linear or branched alkyl;
b) —OR⁹;
R⁹ is hydrogen or C₁-C₄ linear alkyl; and
c) —N(R¹⁰a)(R^{10b});
R^{10a} and R^{10b} are each independently hydrogen or C₁-C₄ linear alkyl;
vii) —OC(O)R¹¹; R¹¹ is C₁-C₄ linear or branched alkyl or phenyl;
viii) —CN;
ix) —NO₂;
x) —SO₂R¹⁶; R¹⁶ is hydrogen, hydroxyl, or C₁-C₄ linear or branched alkyl; and
xi) halogen; and
each R^{b} is independently chosen from:
i) C₁-C₄ linear, branched, or cyclic alkyl;
ii) C₁-C₄ haloalkyl;
iii) phenyl;
iv) —OR²⁵;
wherein R²⁵ is chosen from:
a) —H; and
b) C₁-C₄ linear or branched alkyl or C₁-C₄ linear or branched haloalkyl;
v) —N(R^{26a})(R^{26b});
wherein R^{26a} and R^{26b} are each independently chosen from:
a) —H; and
b) C₁-C₄ linear or branched alkyl;
vi) —C(O)R²⁸;
wherein R²⁸ is chosen from:
a) C₁-C₄ linear or branched alkyl;
b) —OR²⁸;
R²⁸ is hydrogen or C₁-C₄ linear alkyl; and
c) —N(R^{30a})(R^{30b});
R^{30a} and R^{30b} are each independently hydrogen or C₁-C₄ linear alkyl;
vii) —OC(O)R³¹; R³¹ is C₁-C₄ linear or branched alkyl or phenyl;
viii) —CN;
ix) —NO₂;
x) —SO₂R³⁶; R³⁶ is hydrogen, hydroxyl, or C₁-C₄ linear or branched alkyl; and
xi) halogen.

26. The use according to claim 21, wherein the compound has the formula: 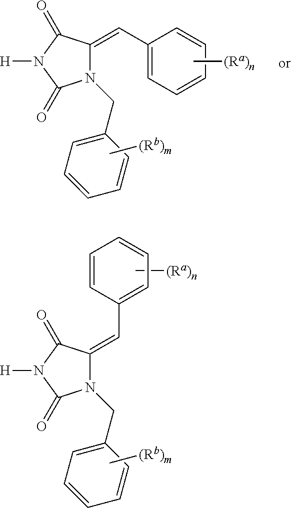
wherein the indices m and n are each independently from 1 to 5;
each R^{a} is independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃; and
each R^{b} is independently chosen from:
i) —CH₃;
ii) —C₂H₅;
iii) —F;
iv) —Cl;
v) —Br;
vi) —OH;
vii) —OCH₃;
viii) —OC₂H₅;
ix) —OC₃H₇;
x) —OCH(CH₃)₂;
xi) —CF₃;
xii) —OCF₃;
xii) —OCF₂CHF₂;
xiii) —COCH₃;
xiv) —COC₆H₅;
xv) —CN;
xvi) —C₆H₅;
xvii) —N(CH₃)₂; and
xviii) —SO₂CH₃.

27. The use of a compound, or a pharmaceutically acceptable salt thereof, for treating cancer, wherein the cancer is chosen from brain, squamous cell, bladder, gastric, pancreatic, breast, head, neck, oesophageal, prostate, colorectal, lung, renal, kidney, ovarian, gynecological, thyroid cancer, and hematologic cancer comprising administering to a human an effective amount of one or more compounds having the formula: 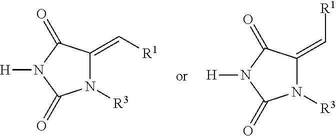
wherein R¹ is phenyl or phenyl substituted by from 1 to 5 organic radicals; and
R³ is benzyl or benzyl substituted by from 1 to 5 organic radicals.

28. The use of a compound for treating a hyperproliferative disease, comprising administering to a human an effective amount of one or more compounds, or a pharmaceutically acceptable salt thereof, having the formula: 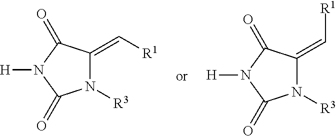
wherein R¹ is phenyl or phenyl substituted by from 1 to 5 organic radicals; R³ is benzyl or benzyl substituted by from 1 to 5 organic radicals.

29. The use of a composition for treating prostate cancer, comprising administering to a human an effective amount of a composition comprising:
a) a therapeutically effective amount of one or more compounds, or a pharmaceutically acceptable salt thereof, having the formula: 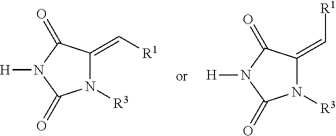
wherein R¹ is phenyl or phenyl substituted by from 1 to 5 organic radicals;
R³ is benzyl or benzyl substituted by from 1 to 5 organic radicals; and
b) one or more pharmaceutically acceptable carriers.

30. The use of a composition for treating prostate cancer, comprising administering to a human an effective amount of a composition comprising:
a) a therapeutically effective amount of one or more compounds, or a pharmaceutically acceptable salt thereof, having the formula: 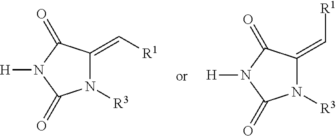
wherein R¹ is phenyl or phenyl substituted by from 1 to 5 organic radicals;
R³ is benzyl or benzyl substituted by from 1 to 5 organic radicals; and
b) an effective amount of one or more mTOR inhibitors.

31. The use of a combination of medicaments for treating prostate cancer, comprising administering to a human an effective amount of a medicament chosen from:
a) a therapeutically effective amount of one or more compounds, or a pharmaceutically acceptable salt thereof, having the formula: 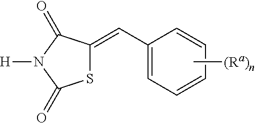
R^{a} is from 1 to 5 organic radicals that are substitutions for hydrogen; and
b) an effective amount of one or more mTOR inhibitors;
wherein if administered sequentially, the administration can be in any order.

32. The use of a combination of medicaments for treating prostate cancer, comprising administering to a human an effective amount of a medicament chosen from:
a) a therapeutically effective amount of one or more compounds having the formula: 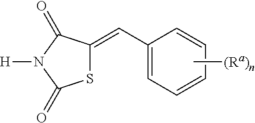
R^{a} is from 1 to 5 organic radicals that are substitutions for hydrogen; and
b) an effective amount of one or more mTOR inhibitors;
wherein if the administered sequentially, the administration can be in any order.

33. The use of a medicament for treating prostate cancer, comprising administering to a human an effective amount of a medicament comprising:
a) a therapeutically effective amount of one or more compounds having the formula: 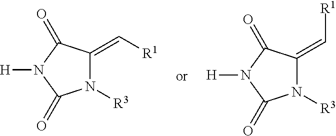
wherein R¹ is phenyl or phenyl substituted by from 1 to 5 organic radicals;
R³ is benzyl or benzyl substituted by from 1 to 5 organic radicals; and
b) an effective amount of rapamycin.

34. The use of a combination of medicaments for treating prostate cancer, comprising administering to a human an effective amount of a medicament chosen from:
a) a therapeutically effective amount of one or more compounds, or a pharmaceutically acceptable salt thereof, having the formula: 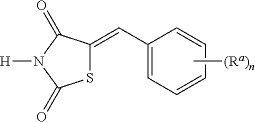
R^{a} is from 1 to 5 organic radicals that are substitutions for hydrogen; and
b) an effective amount of rapamycin;
wherein if administered sequentially, the administration can be in any order.

35. The use of a combination of medicaments for treating prostate cancer, comprising administering to a human an effective amount of a medicament chosen from:
a) a therapeutically effective amount of one or more compounds, or a pharmaceutically acceptable salt thereof, having the formula: 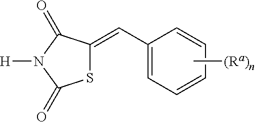
R^{a} is from 1 to 5 organic radicals that are substitutions for hydrogen; and
b) an effective amount of rapamycin;
wherein if the administered sequentially, the administration can be in any order.

36. The use of a compound for inhibiting Pim-1 and/or Pim-2 in vitro, comprising contacting Pim-1 and/or Pim-2 with an effective amount of one or more compounds, or a pharmaceutically acceptable salt thereof, having the formula: 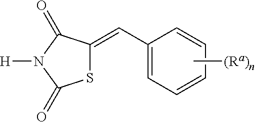
wherein R^{a} is from 1 to 5 organic radicals that are substitutions for hydrogen.

37. The use of a compound for inhibiting Pim-1 and/or Pim-2 ex vivo, comprising contacting a cell with an amount effective for inhibiting Pim-1 with one or more compounds, or pharmaceutically acceptable salts thereof, having the formula: 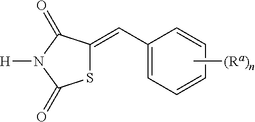
wherein R^{a} is from 1 to 5 organic radicals that are substitutions for hydrogen.

38. The use of a compound for inhibiting Pim-1 and/or Pim-2 in vivo, comprising contacting a cell with an amount effective for inhibiting Pim-1 with one or more compounds, or pharmaceutically acceptable salts thereof, having the formula: 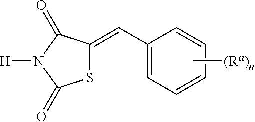
wherein R^{a} is from 1 to 5 organic radicals that are substitutions for hydrogen.

39. A composition comprising:
a) an effective amount of one or more compounds, or a pharmaceutically acceptable salt thereof, having the formula: 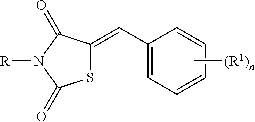
wherein R is chosen from:
i) hydrogen;
ii) C₁-C₄ linear, branched, or cyclic alkyl;
R¹ is an organic radical that can substituted for a hydrogen atom; and
the index n is from 0 to 5; and
b) one or more excipients.

40. The use of a combination of medicaments for treating prostate cancer, comprising administering to a human an effective amount of a medicament chosen from:
a) a therapeutically effective amount of one or more compounds, or a pharmaceutically acceptable salt thereof, having the formula: 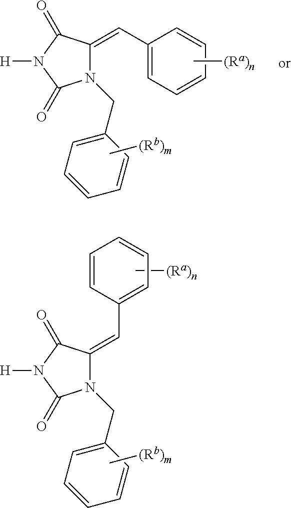
wherein the indices m and n are each independently from 1 to 5;
R^{a} is from 1 to 5 independently chosen organic radicals that are substitutions for hydrogen; and
R^{b} is from 1 to 5 independently chosen organic radicals that are substitutions for hydrogen; and
b) an effective amount of rapamycin;
wherein if administered sequentially, the administration can be in any order.

41. The use of a combination of medicaments for treating prostate cancer, comprising administering to a human an effective amount of a medicament chosen from:
a) a therapeutically effective amount of one or more compounds, or a pharmaceutically acceptable salt thereof, having the formula: 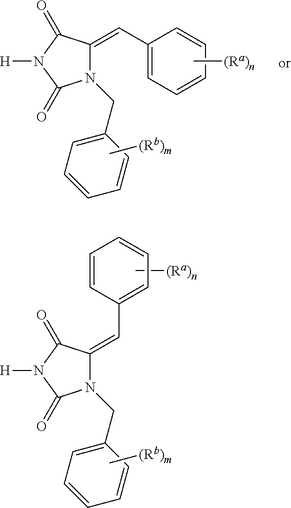
wherein the indices m and n are each independently from 1 to 5;
R^{a} is from 1 to 5 independently chosen organic radicals that are substitutions for hydrogen; and
R^{b} is from 1 to 5 independently chosen organic radicals that are substitutions for hydrogen; and
b) an effective amount of rapamycin;
wherein if the administered sequentially, the administration can be in any order.

42. The use of a compound for inhibiting Pim-1 and/or Pim-2 in vitro, comprising contacting Pim-1 with an effective amount of one or more compounds, or a pharmaceutically acceptable salt thereof, having the formula: 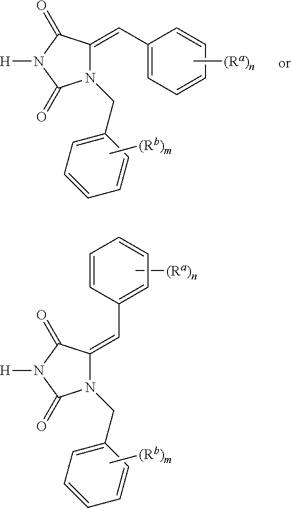
wherein the indices m and n are each independently from 1 to 5;
R^{a} is from 1 to 5 independently chosen organic radicals that are substitutions for hydrogen; and
R^{b} is from 1 to 5 independently chosen organic radicals that are substitutions for hydrogen.

43. The use of a compound for inhibiting Pim-1 and/or Pim-2 ex vivo, comprising contacting a cell with an amount effective for inhibiting Pim-1 and/or Pim-2 with one or more compounds, or a pharmaceutically acceptable salt thereof, having the formula: 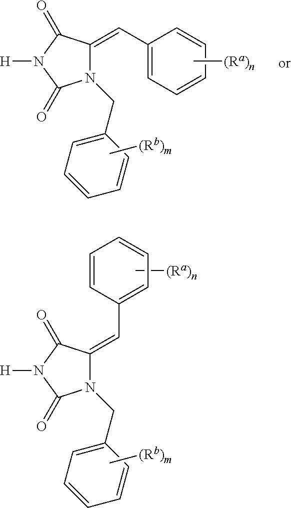
wherein the indices m and n are each independently from 1 to 5;
R^{a} is from 1 to 5 independently chosen organic radicals that are substitutions for hydrogen; and
R^{b} is from 1 to 5 independently chosen organic radicals that are substitutions for hydrogen.

44. The use of a compound for inhibiting Pim-1 and/or Pim-2 in vivo, comprising contacting a cell with an amount effective for inhibiting Pim-1 and/or Pim-2 with one or more compounds, or a pharmaceutically acceptable salt thereof, having the formula: 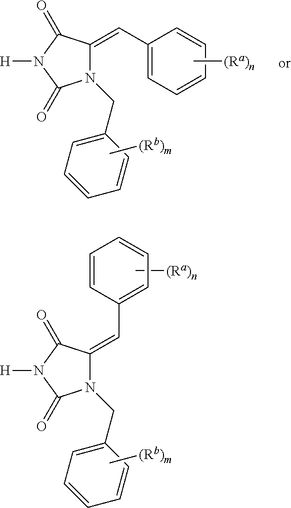
wherein the indices m and n are each independently from 1 to 5;
R^{a} is from 1 to 5 independently chosen organic radicals that are substitutions for hydrogen; and
R^{b} is from 1 to 5 independently chosen organic radicals that are substitutions for hydrogen.

45. A composition comprising:
a) an effective amount of one or more compounds, or a pharmaceutically acceptable salt thereof, having the formula: 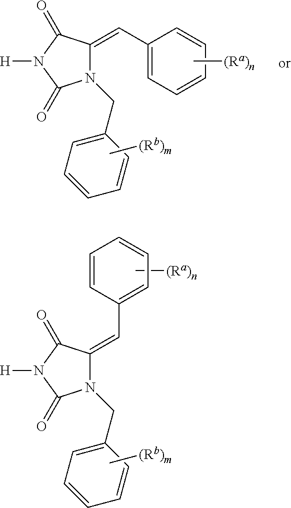
wherein the indices m and n are each independently from 1 to 5;
R^{a} is from 1 to 5 independently chosen organic radicals that are substitutions for hydrogen; and
R^{b} is from 1 to 5 independently chosen organic radicals that are substitutions for hydrogen; and
b) one or more excipients.

46. A compound, or a pharmaceutically acceptable salt thereof, having the formula: 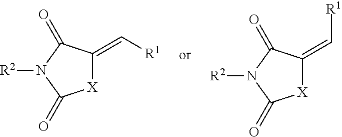
wherein
X is S or NR³;
R³ is benzyl or benzyl substituted by from 1 to 5 organic radicals;
R¹ is phenyl substituted by from 1 to 5 organic radicals wherein at least one radical is haloalkyl or haloalkoxy; and
R² is chosen from:
i) hydrogen;
ii) C₁-C₄ linear, branched, or cyclic alkyl; and
iii) benzyl or benzyl substituted by from 1 to 5 organic radicals;
with the proviso the compound is not:
5-(3-trifluoromethylbenzylidene)thiazolidine-2,4-dione;
5-(3-trifluoromethoxybenzylidene)thiazolidine-2,4-dione; or
5-(4-trifluoromethylbenzylidene)thiazolidine-2,4-dione.

47. The compound according to claim 46, wherein the at least one organic radical is chosen from —CH₂F, —CHF₂, —CF₃, —CH₂CH₂F, —CH₂CHF₂, —CH₂CF₃, —CHFCH₃, —CF₂CH₃, —CHFCH₂F, —CF₂CH₂F, —CF₂CHF₂, and —CF₂CF₃.

48. A compound according to claim 46, wherein the at least one organic radical is chosen from —OCH₂F, —OCHF₂, —OCF₃, —OCH₂CH₂F, —OCH₂CHF₂, —OCH₂CF₃, —OCHFCH₃, —OCF₂CH₃, —OCHFCH₂F, —OCF₂CH₂F, —OCF₂CHF₂, and —OCF₂CF₃.

49. A compound according to claim 46, wherein the at least one organic radical is chosen from —CH₂Cl, —CHCl₂, —CCl₃, —CH₂CH₂Cl, —CH₂CHCl₂, —CH₂CCl₃, —CHClCH₃, —CCl₂CH₃, —CHClCH₂Cl, —CCl₂CH₂Cl, —CCl₂CHCl₂, and —CCl₂CCl₃.

50. A compound according to claim 46, wherein the at least one organic radical is chosen from —OCH₂Cl, —OCHCl₂, —OCCl₃, —OCH₂CH₂Cl, —OCH₂CHCl₂, —OCH₂CCl₃, —OCHClCH₃, —OCCl₂CH₃, —OCHClCH₂Cl, —OCCl₂CH₂Cl, —OCCl₂CHCl₂, and —OCCl₂CCl₃.

51. A compound according to claim 46, having the formula: 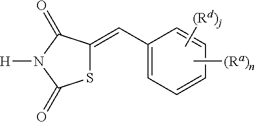
wherein R^{a} is an organic radical;
R^{d} is an organic radical chosen from haloalkyl and haloalkoxy;
the index n is from 0 to 4; and
the index j is from 1 to 5.

52. A compound according to claim 46, having the formula: 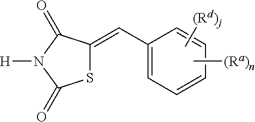
wherein the index n is from 0 to 4;
the index j is from 1 to 5;
R^{a} is from 0 to 4 organic radicals that are substitutions for hydrogen independently chosen from:
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{4a})(R^{4b})]_{y}OR⁵;
wherein R⁵ is chosen from:
a) —H;
b) C₁-C₁₂ substituted or unsubstituted linear, branched, or cyclic alkyl or C₁-C₁₂ substituted or unsubstituted linear, branched, or cyclic haloalkyl;
c) C₆ or C₁₀ substituted or unsubstituted aryl or C₇-C₂₀ alkylenearyl;
d) C₁-C₉ substituted or unsubstituted heterocyclic; and
e) C₁-C₁₁ substituted or unsubstituted heteroaryl;
viii) —[C(R^{4a})(R^{4b})]_{y}N(R^{6a})(R^{6b});
wherein R^{6a} and R^{6b} are each independently chosen from:
a) —H;
b) —OR⁷;
R⁷ is hydrogen or C₁-C₄ linear alkyl;
c) C₁-C₁₂ substituted or unsubstituted linear, branched, or cyclic alkyl;
d) C₆ or C₁₀ substituted or unsubstituted aryl;
e) C₁-C₉ substituted or unsubstituted heterocyclic;
f) C₁-C₁₁ substituted or unsubstituted heteroaryl; and
g) R^{6a} and R^{6b} can be taken together to form a substituted or unsubstituted ring having from 3 to 10 carbon atoms and from 0 to 3 heteroatoms chosen from oxygen, nitrogen, and sulfur;
ix) —[C(R^{4a})(R^{4b})]_{y}C(O)R⁸;
wherein R⁸ is chosen from:
a) C₁-C₁₂ substituted or unsubstituted linear, branched, or cyclic alkyl;
b) —OR⁹;
wherein R⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl; and
c) —N(R^{10a})(R^{10b});
wherein R^{10a} and R^{10b} are each independently hydrogen, C₁-C₁₂ substituted or unsubstituted linear, branched, or cyclic alkyl; C₆ or C-₁₀ substituted or unsubstituted aryl; C₁-C₉ substituted or unsubstituted heterocyclic; C₁-C₁₁ substituted or unsubstituted heteroaryl; or R^{10a} and R^{10b} can be taken together to form a substituted or unsubstituted ring having from 3 to 10 carbon atoms and from 0 to 3 heteroatoms chosen from oxygen, nitrogen, and sulfur;
x) —[C(R^{4a})(R^{4b})]_{y}OC(O)R¹¹;
wherein R¹¹ is chosen from:
a) C₁-C₁₂ substituted or unsubstituted linear, branched, or cyclic alkyl; and)
b) —N(R^{12a})(R^{12b});
R^{12a} and R^{12b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{12a} and R^{12b} can be taken together to form a substituted or unsubstituted ring having from 3 to 10 carbon atoms and from 0 to 3 heteroatoms chosen from oxygen, nitrogen, and sulfur;
xi) —[C(R^{4a})(R^{4b})]_{y}NR¹³C(O)R¹⁴;
wherein R¹³ is chosen from:
a) —H; and
b) C₁-C₄ substituted or unsubstituted linear, branched, or cyclic alkyl;
wherein R¹⁴ is chosen from:
a) C₁-C₁₂ substituted or unsubstituted linear, branched, or cyclic alkyl; and
b) —N(R^{15a})(R^{15b});
R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
xii) —[C(R^{4a})(R^{4b})]_{y}CN;
xiii) —[C(R^{4a})(R^{4b})]_{y}NO₂;
xiv) —[C(R^{4a})(R^{4b})]_{y}SO₂R¹⁶;
R¹⁶ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl; and
xv) halogen;
R^{4a} and R^{4b} are each independently hydrogen or C₁-C₄ alkyl; and
the index y is from 0 to 5;
each R^{d} is independently an organic radical having the formula:
—[C(H)ₐ(Z)_{b}]_{d}C(H)ₑ(Z)_{f} or —O[C(H)ₐ(Z)_{b}]_{d}C(H)ₑ(Z)_{f}
Z is halogen;
the index a is from 0 to 2; the index b is from 0 to 2; the index d is from 0 to 6; the index e is from 0 to 3; the index f is from 0 to 3; with the proviso that the indices b and f are not both equal to 0.

53. The compound according to claim 46, wherein the compounds are salts comprising anions chosen from chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, and citrate.

54. The compound according to claim 46, wherein the compounds are salts comprising cations chosen from sodium, lithium, potassium, calcium, magnesium, and bismuth.

55. A compound chosen from:
5-[3-(1-fluoroethoxy)benzylidene]thiazolidine-2,4-dione;
5-[3-(1,1-difluoroethoxy)benzylidene]thiazolidine-2,4-dione;
5-[3-(1,1,2-trifluoroethoxy)benzylidene]thiazolidine-2,4-dione;
5-[3-(1,1,2,2-tetrafluoroethoxy)benzylidene]thiazolidine-2,4-dione
5-[3-(1,1,2,2,2-pentafluoroethoxy)benzylidene]thiazolidine-2,4-dione;
5-[3-(2-fluoroethoxy)benzylidene]thiazolidine-2,4-dione;
5-[3-(2,2-difluoroethoxy)benzylidene]thiazolidine-2,4-dione;
5-[3-(2,2,2-trifluoroethoxy)benzylidene]thiazolidine-2,4-dione;
5-[3-(1,2,2,2-tetrafluoroethoxy)benzylidene]thiazolidine-2,4-dione;
5-[4-(1-fluoroethoxy)benzylidene]thiazolidine-2,4-dione;
5-[4-(1,1-difluoroethoxy)benzylidene]thiazolidine-2,4-dione;
5-[4-(1,1,2-trifluoroethoxy)benzylidene]thiazolidine-2,4-dione;
5-[4-(1,1,2,2-tetrafluoroethoxy)benzylidene]thiazolidine-2,4-dione
5-[4-(1,1,2,2,2-pentafluoroethoxy)benzylidene]thiazolidine-2,4-dione;
5-[4-(2-fluoroethoxy)benzylidene]thiazolidine-2,4-dione;
5-[4-(2,2-difluoroethoxy)benzylidene]thiazolidine-2,4-dione;
5-[4-(2,2,2-trifluoroethoxy)benzylidene]thiazolidine-2,4-dione;
5-[4-(1,2,2,2-tetrafluoroethoxy)benzylidene]thiazolidine-2,4-dione;
5-(2-trifluoromethoxybenzylidene)thiazolidine-2,4-dione
5-(4-trifluoromethoxybenzylidene)thiazolidine-2,4-dione;
5-(2-trifluoromethylbenzylidene)thiazolidine-2,4-dione;
5-[2,3-di(trifluoromethyl)benzylidene]thiazolidine-2,4-dione;
5-[2,4-di(trifluoromethyl)benzylidene]thiazolidine-2,4-dione;
5-[2,5-di(trifluoromethyl)benzylidene]thiazolidine-2,4-dione;
5-[2,6-di(trifluoromethyl)benzylidene]thiazolidine-2,4-dione;
5-[2,3-di(trifluoromethoxy)benzylidene]thiazolidine-2,4-dione;
5-[2,4-di(trifluoromethoxy)benzylidene]thiazolidine-2,4-dione;
5-[2,5-di(trifluoromethoxy)benzylidene]thiazolidine-2,4-dione;
5-[2,6-di(trifluoromethoxy)benzylidene]thiazolidine-2,4-dione;
5-[3-fluoro-2-(trifluoromethyl)benzylidene]thiazolidine-2,4-dione;
5-[4-fluoro-2-(trifluoromethyl)benzylidene]thiazolidine-2,4-dione;
5-[5-fluoro-2-(trifluoromethyl)benzylidene]thiazolidine-2,4-dione;
5-[6-fluoro-2-(trifluoromethyl)benzylidene]thiazolidine-2,4-dione;
5-[2-fluoro-3-(trifluoromethyl)benzylidene]thiazolidine-2,4-dione;
5-[4-fluoro-3-(trifluoromethyl)benzylidene]thiazolidine-2,4-dione;
5-[5-fluoro-3-(trifluoromethyl)benzylidene]thiazolidine-2,4-dione;
5-[6-fluoro-3-(trifluoromethyl)benzylidene]thiazolidine-2,4-dione;
5-[2-fluoro-4-(trifluoromethyl)benzylidene]thiazolidine-2,4-dione;
5-[3-fluoro-4-(trifluoromethyl)benzylidene]thiazolidine-2,4-dione;
5-[5-fluoro-4-(trifluoromethyl)benzylidene]thiazolidine-2,4-dione;
5-[6-fluoro-4-(trifluoromethyl)benzylidene]thiazolidine-2,4-dione;
5-[3-fluoro-2-(trifluoromethoxy)benzylidene]thiazolidine-2,4-dione;
5-[4-fluoro-2-(trifluoromethoxy)benzylidene]thiazolidine-2,4-dione;
5-[5-fluoro-2-(trifluoromethoxy)benzylidene]thiazolidine-2,4-dione;
5-[6-fluoro-2-(trifluoromethoxy)benzylidene]thiazolidine-2,4-dione;
5-[2-fluoro-3-(trifluoromethoxy)benzylidene]thiazolidine-2,4-dione;
5-[4-fluoro-3-(trifluoromethoxy)benzylidene]thiazolidine-2,4-dione;
5-[5-fluoro-3-(trifluoromethoxy)benzylidene]thiazolidine-2,4-dione;
5-[6-fluoro-3-(trifluoromethoxy)benzylidene]thiazolidine-2,4-dione;
5-[2-fluoro-4-(trifluoromethoxy)benzylidene]thiazolidine-2,4-dione;
5-[3-fluoro-4-(trifluoromethoxy)benzylidene]thiazolidine-2,4-dione;
5-[5-fluoro-4-(trifluoromethoxy)benzylidene]thiazolidine-2,4-dione; and
5-[6-fluoro-4-(trifluoromethoxy)benzylidene]thiazolidine-2,4-dione.

56. The use of a compound according to claim 46, for stimulating the phosphorylation of multiple substrates of AMPK in vivo, in vitro, or ex vitro.
